(19) United States
(12) Patent Application Publication
(10) Pub. No.: US 2025/0311622 A1
(43) Pub. Date: Oct. 2, 2025

(54) HETEROCYCLIC COMPOUND AND ORGANIC LIGHT EMITTING DEVICE COMPRISING SAME

(71) Applicant: LT MATERIALS CO., LTD., Yongin-City (KR)

(72) Inventors: Na-Gyung LIM, Yongin-City (KR); Nam-Jin LEE, Yongin-City (KR); Won-Jang JEONG, Yongin-City (KR); Dong-Jun KIM, Yongin-City (KR); Dae-Hyuk CHOI, Yongin-City (KR)

(73) Assignee: LT MATERIALS CO., LTD., Yongin-City (KR)

(21) Appl. No.: 19/087,015

(22) Filed: Mar. 21, 2025

(30) Foreign Application Priority Data
Mar. 26, 2024 (KR) 10-2024-0040927

(51) Int. Cl. H10K 85/60; C07D 209/88; C07D 409/12; C07D 409/14; H10K 50/15; H10K 50/18

(52) U.S. Cl. CPC H10K 85/636; C07D 209/88; C07D 409/12; C07D 409/14; H10K 85/633; H10K 50/15; H10K 50/181; H10K 85/615; H10K 85/622; H10K 85/6572; H10K 85/6574

(57) ABSTRACT

Disclosed are a heterocyclic compound of Chemical Formula 1 and an organic light emitting device including the same. When the heterocyclic compound is used for an organic light emitting device, the driving voltage of the device can be lowered, the light efficiency of the device can be improved, and the thermal stability of the heterocyclic compound can be improved to improve the service life characteristics of the device.

### CROSS-REFERENCE TO RELATED APPLICATIONS

This application claims priority to and the benefit of Korean Patent Application No. 10-2024-0040927 filed in the Korean Intellectual Property Office on Mar. 26, 2024, the entire contents of which are incorporated herein by reference.

### TECHNICAL FIELD

The present specification relates to a heterocyclic compound and an organic light emitting device including the same.

### BACKGROUND ART

A light emitting device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute an emission layer alone, or it is also possible to use a compound, which may serve as a host or a dopant of a host-dopant-based emission layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENTS

### Patent Documents

(Patent Document 1) U.S. Pat. No. 4,356,429

### SUMMARY OF THE INVENTION

The present invention has been made in an effort to provide a heterocyclic compound and an organic light emitting device including the same.

An exemplary embodiment of the present invention provides a heterocyclic compound represented by the following Chemical Formula 1.

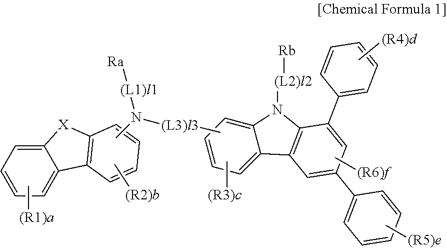

In Chemical Formula 1,
X is O; or S,
Ra and Rb are the same as or different from each other, and are each independently a substituted or unsubstituted C6 to C60 aryl group; or a substituted or unsubstituted C2 to C60 heteroaryl group,
L1 to L3 are the same as or different from each other, and are each independently a direct bond; or a substituted or unsubstituted C6 to C60 arylene group,
each of l1 to l3 is an integer from 0 to 4, and
when each of l1 to l3 is an integer of 2 or higher, substituents in the parenthesis are the same as or different from each other,
R1 to R6 are the same as or different from each other, and are each independently hydrogen; deuterium; a cyano group; a halogen group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted silyl group; or a substituted or unsubstituted phosphine oxide group,
a is an integer from 0 to 4,
each of b and c is an integer from 0 to 3,
each of d and e is an integer from 0 to 5,
f is an integer from 0 to 2, and
when each of a, b, c, d, e, and f is 2 or an integer higher than 2, substituents in the parenthesis are the same as or different from each other.

Another exemplary embodiment provides an organic light emitting device including: a first electrode; a second electrode disposed to face the first electrode; and an organic material layer having one or more layers disposed between the first electrode and the second electrode, in which one or more layers of the organic material layer include the above-described heterocyclic compound.

The heterocyclic compound according to an exemplary embodiment of the present application is a tertiary amine represented by Chemical Formula 1, in which a first substituent is a tricyclic heteroaryl group including a heteroatom O or S (represented by X) and is directly linked to the N atom of the amine, a second substituent has an aryl group or heteroaryl group composed of a terminal group Ra linked to the N atom of the amine via a linker (represented by L1), and a third substituent has a terminal carbazole group that is linked to the N atom of the amine via a linker (represented by L3), and two phenyl rings are linked to specific positions on the benzene ring on the other side of the benzene ring linked to the N atom around the carbazole group.

As represented by Chemical Formula 1 above, the first substituent adjusts the hole transport rate, the second substituent adjusts the structural stabilization and charge balance, and the third substituent adjusts the hole transport rate of carbazole, which has strong hole characteristics, making the present invention usable as an electron blocking layer and a hole transport layer.

As a result, when the heterocyclic compound is used for an organic light emitting device, the driving voltage of the device can be lowered, the light efficiency of the device can be improved, and the thermal stability of the heterocyclic compound can be improved to improve the service life characteristics of the device.

### BRIEF DESCRIPTION OF THE DRAWINGS

FIGS. 1 to 5 are views each exemplarily illustrating a stacking structure of an organic light emitting device according to an exemplary embodiment of the present specification.

### DETAILED DESCRIPTION

Hereinafter, the present specification will be described in more detail.

### Definitions

When one part “includes” one constituent element in the present specification, unless otherwise specifically described, this does not mean that another constituent element is excluded, but means that another constituent element may be further included.

In the present specification,

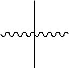

of a chemical formula or structural formula means a position to be bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means to be unsubstituted or substituted with one or more substituents selected from the group consisting of deuterium; a cyano group; a halogen group; a C1 to C60 straight-chained or branched alkyl group; a C2 to C60 straight-chained or branched alkenyl group; a C2 to C60 straight-chained or branched alkynyl group; a C3 to C60 monocyclic or polycyclic cycloalkyl group; a C2 to C60 monocyclic or polycyclic heterocycloalkyl group; a C6 to C60 monocyclic or polycyclic aryl group; a C2 to C60 monocyclic or polycyclic heteroaryl group; a silyl group; a phosphine oxide group; and an amine group, or to be unsubstituted or substituted with a substituent to which two or more substituents selected from among the exemplified substituents are linked.

In the present specification, “when a substituent is not indicated in the structure of a chemical formula or compound” means that a hydrogen atom is bonded to a carbon atom. However, since deuterium (²H) or tritium corresponds to an isotope of hydrogen, it may be interpreted as a concept included in hydrogen, as long as it is not explicitly excluded.

That is, in the present application, deuterium exhibits an effect equivalent to that of hydrogen in terms of driving voltage, light emitting efficiency, and service life, or exhibits improved effects in some evaluation criteria, according to Chem. Commun., 2014, 50, 14870, and since the effect falls within the scope that a person with ordinary skill in the art may be predicted to have the equivalent effect without conducting specific experiments, deuterium, an isotope of hydrogen, is interpreted as a concept included in hydrogen, as long as it is not explicitly excluded.

According to an exemplary embodiment of the present specification, “when a substituent is not indicated in the structure of a chemical formula or compound” may mean that all the positions that may be reached by the substituent are hydrogen; or deuterium. That is, deuterium is an isotope of hydrogen, and some hydrogen atoms may be deuterium which is an isotope, and in this case, the content of deuterium may be 0% to 100%.

According to an exemplary embodiment of the present specification, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

According to an exemplary embodiment of the present specification, deuterium is one of the isotopes of hydrogen, is an element that has a deuteron composed of one proton and one neutron as a nucleus, and may be represented by hydrogen-2, and the element symbol may also be expressed as D or ²H.

According to an exemplary embodiment of the present specification, the isotope means an atom with the same atomic number (Z), but different mass numbers (A), and may also be interpreted as an element which has the same number of protons, but different number of neutrons.

According to an exemplary embodiment of the present specification, when the total number of substituents of a basic compound is defined as T1 and the number of specific substituents among the substituents is defined as T2, the content T % of the specific substituent may be defined as T2/T1×100=T %.

That is, when taking a phenyl group represented by

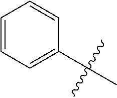 [SMILES: *c1ccccc1]

as an example, herein, a deuterium content of 20% may be represented by 20% when the total number of substituents that the phenyl group can have is 5 (T1 in the formula) and the number of deuterium atoms among the substituents is 1 (T2 in the formula). That is, a deuterium content of 20% in the phenyl group may be represented by the following structural formula.

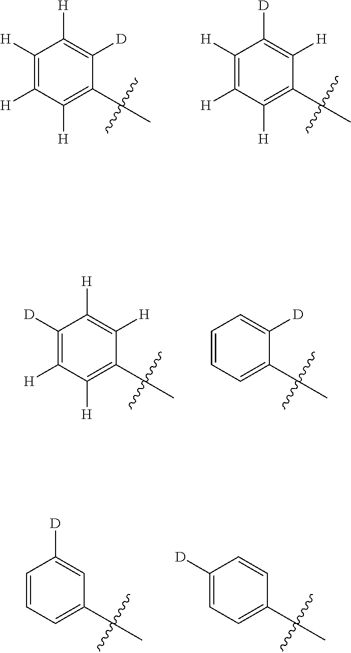 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, according to an exemplary embodiment of the present specification, “a phenyl group having a deuterium content of 0%” may mean a phenyl group that does not include a deuterium atom, that is, has five hydrogen atoms.

In the present specification, the cyano group may mean —CN.

In the present specification, the halogen group may be fluorine, chlorine, bromine, or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group; a 2-butenyl group; a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

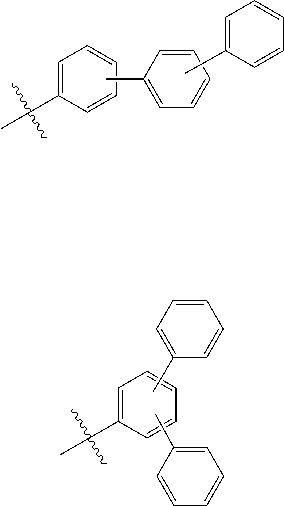

In the present specification, the fluorenyl group may be substituted, and adjacent substituents may be bonded to each other to form a ring.

When the fluorenyl group is substituted, the substituent may be selected from the following structures, but is not limited thereto.

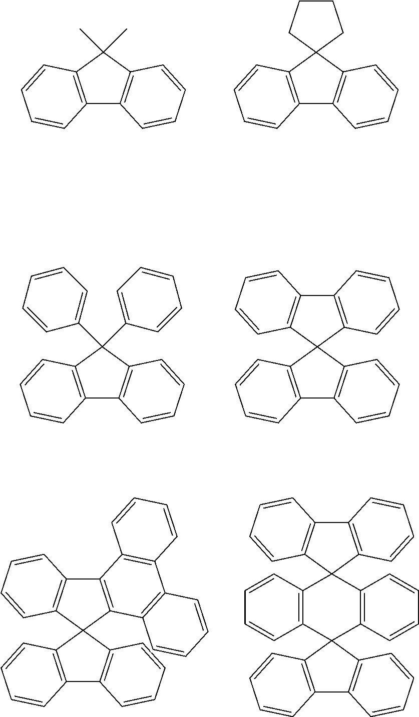

In the present specification, the heteroaryl group includes S, O, Se, N, or Si as a heteroatom, includes a monocycle or a polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, a 2,3-dihydrobenzo[b]thiophene group, a 2,3-dihydrobenzofuran group, a 5,10-dihydrodibenzo[b,e][1,4]azasilinyl group, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, when the substituent is a carbazole group, it means being bonded to nitrogen or carbon of carbazole.

In the present specification, when a carbazole group is substituted, an additional substituent may be substituted with the nitrogen or carbon of the carbazole.

In the present specification, a benzocarbazole group may be any one of the following structures.

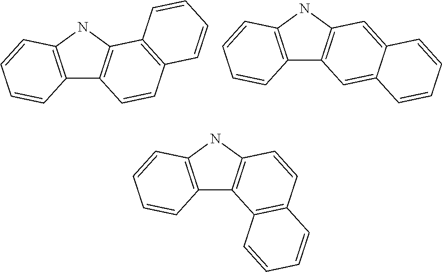

In the present specification, a dibenzocarbazole group may be any one of the following structures.

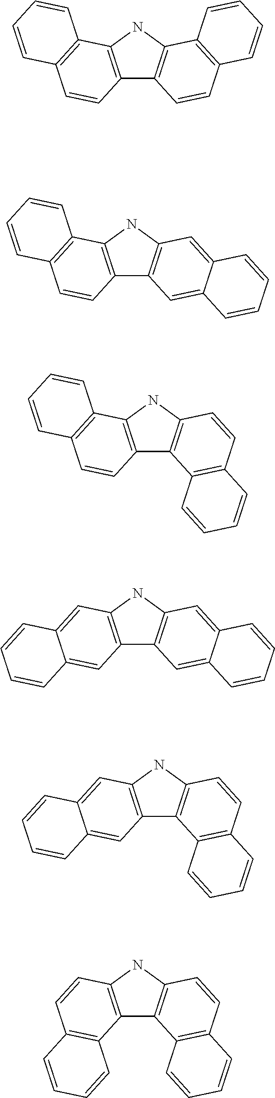 [SMILES: c1ccc2c(c1)ccc1c3ccc4ccccc4c3[nH]c21 ; c1ccc2cc3c(cc2c1)[nH]c1c2ccccc2ccc31 ; c1ccc2c(c1)ccc1c2[nH]c2ccc3ccccc3c21 ; c1ccc2cc3c(cc2c1)[nH]c1cc2ccccc2cc13 ; c1ccc2cc3c(cc2c1)[nH]c1ccc2ccccc2c13 ; c1ccc2c(c1)ccc1[nH]c3ccc4ccccc4c3c12]

In the present specification, a naphthobenzofuran group may be any one of the following structures.

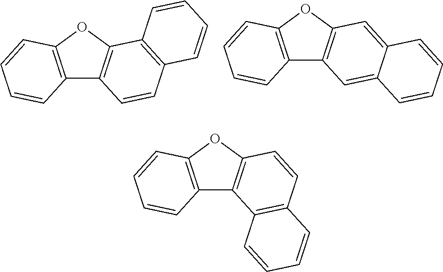

In the present specification, a naphthobenzothiophene group may be any one of the following structures.

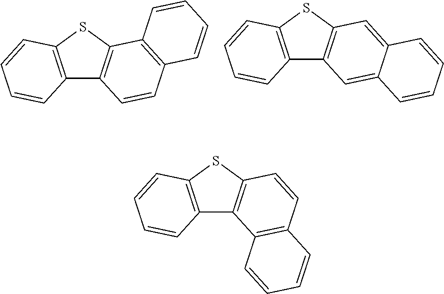

In the present specification, a silyl group includes Si and is a substituent to which the Si atom is directly linked as a radical, and is represented by —Si(R101) (R102) (R103), and R101 to R103 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

Specific examples of the silyl group include

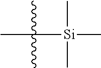 [SMILES: *[Si](C)(C)C]

(a trimethylsilyl group),

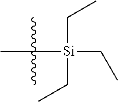 [SMILES: *[Si](CC)(CC)CC]

(a triethylsilyl group),

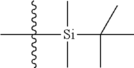 [SMILES: *[Si](C)(C)C(C)(C)C]

(a t-butyldimethylsilyl group),

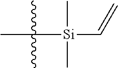 [SMILES: *[Si](C)(C)C=C]

(a vinyldimethylsilyl group),

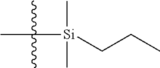 [SMILES: *[Si](C)(C)CCC]

(a propyldimethylsilyl group),

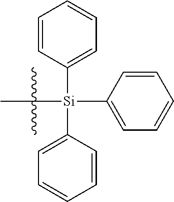 [SMILES: *[Si](c1ccccc1)(c1ccccc1)c1ccccc1]

(a triphenylsilyl group),

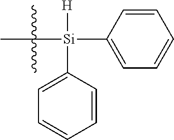 [SMILES: *[SiH](c1ccccc1)c1ccccc1]

(a diphenylsilyl group),

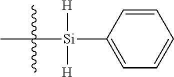 [SMILES: *[SiH2]c1ccccc1]

(a phenylsilyl group) and the like, but are not limited thereto.

In the present specification, the phosphine oxide group is represented by —P(═O)(R104)(R105), and R104 and R105 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, a dinaphthylphosphine oxide group, and the like, but are not limited thereto.

In the present specification, the amine group is represented by —N(R106) (R107), and R106 and R107 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described examples of the aryl group may be applied to an arylene group except for a divalent arylene group.

In the present specification, the above-described examples of the heteroaryl group may be applied to a heteroarylene group except for a divalent heteroarylene group.

In the present specification, an “adjacent” group may mean a substituent substituting an atom directly linked to an atom substituted by the corresponding substituent, a substituent sterically most closely positioned to the corresponding substituent, or another substituent substituting an atom substituted by the corresponding substituent. For example, two substituents substituted at the ortho position in a benzene ring and two substituents substituted at the same carbon in an aliphatic ring may be interpreted as groups which are “adjacent” to each other.

Hydrocarbon rings and hetero rings that adjacent groups may form include an aliphatic hydrocarbon ring, an aromatic hydrocarbon ring, an aliphatic hetero ring and an aromatic hetero ring, and structures exemplified by the above-described cycloalkyl group, aryl group, heterocycloalkyl group and heteroaryl group may be each applied to the rings, except for those that are not monovalent groups.

In an exemplary embodiment of the present specification, a group not represented by a substituent; or a group represented by hydrogen may mean a group all substitutable with deuterium. That is, it may be shown that hydrogen (H); or deuterium (D) can be substituted with each other.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy.

Further, the single bond dissociation energy of carbon and deuterium is higher than the single bond dissociation energy of carbon and hydrogen. Accordingly, the deuterium-substituted structure has an effect of increasing the thermal stability of the molecule and improving the service life of the device using the increased thermal stability.

When a compound is deposited on a silicon wafer, a material including deuterium tends to be packed so that the intermolecular distance is reduced. Further, when the surface of a thin film is observed using an atomic force microscope (AFM), it can be confirmed that the thin film made of a compound including deuterium is deposited with a more uniform surface without any aggregated portion.

### <Heterocyclic Compound>

Hereinafter, the heterocyclic compound according to the present specification will be described.

The heterocyclic compound according to an exemplary embodiment of the present specification may be represented by the following Chemical Formula 1.

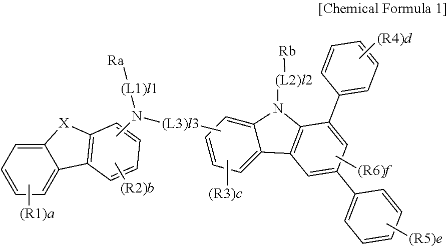

In Chemical Formula 1, the description of each substituent is the same as that described above.

The heterocyclic compound represented by the above formula 1 is a tertiary amine, and includes a first substituent

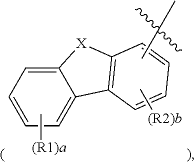

a second substituent

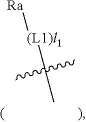

and a third substituent

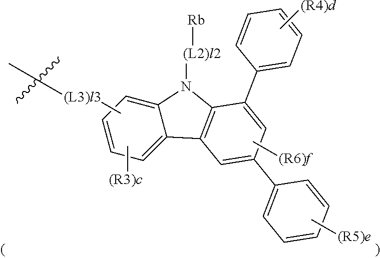

around N of the amine.

As represented by Chemical Formula 1, the first substituent controls the hole transport rate, the second substituent adjusts the structural stabilization and charge balance, and the third substituent controls the hole transport rate of carbazole, which has strong hole characteristics. Overall, the heterocyclic compound serves to improve the electron blocking and/or hole transport characteristics when used in an electron blocking layer and/or hole transport layer.

According to an exemplary embodiment of the present specification, Chemical Formula 1 may be represented by any one of the following Chemical Formulae 1-1 to 1-4.

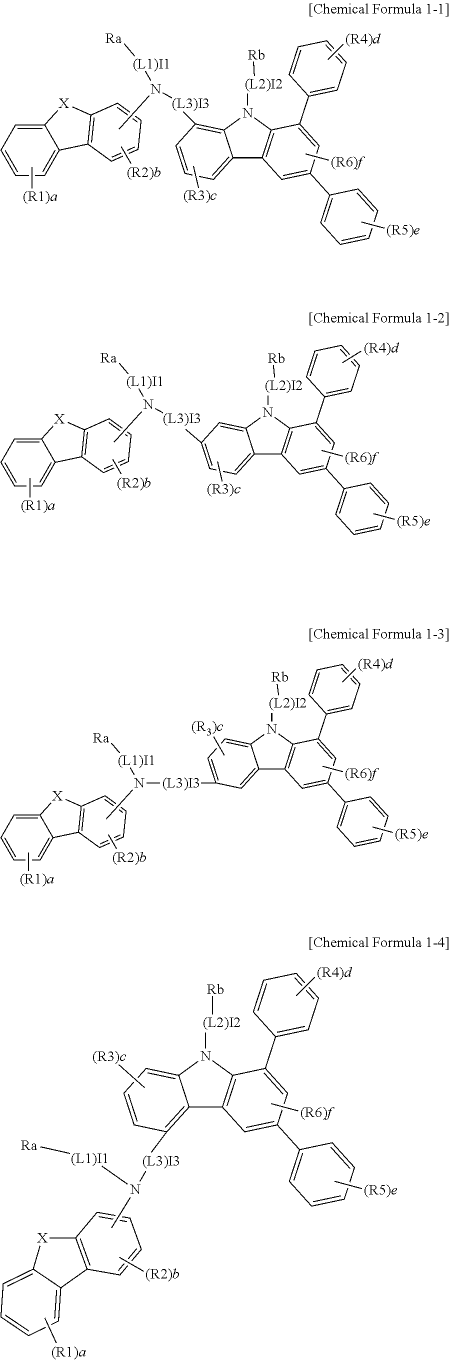

In Chemical Formulae 1-1 to 1-4, each of X, Ra, Rb, L1 to L3, l1 to l3, R1 to R6, a, b, c, d, e, and f is the same as that defined in Chemical Formula 1.

According to an exemplary embodiment of the present specification, Chemical Formula 1-1 may be represented by any one of the following Chemical Formulae 1-11 to 1-14.

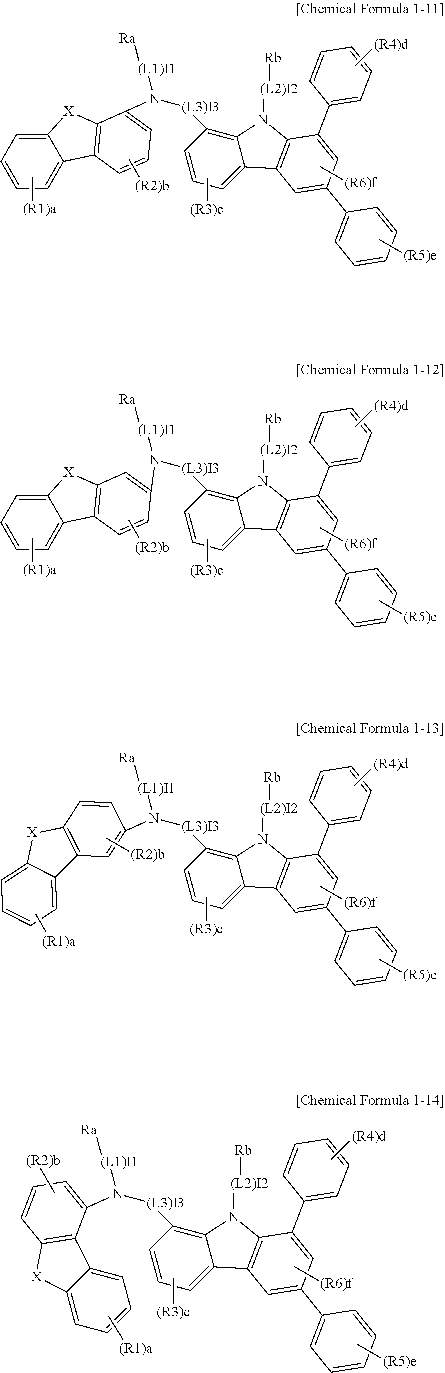

In Chemical Formulae 1-11 to 1-14, the description of each substituent is the same as that described above.

According to an exemplary embodiment of the present specification, Chemical Formula 1-2 may be represented by any one of the following Chemical Formulae 1-21 to 1-24.

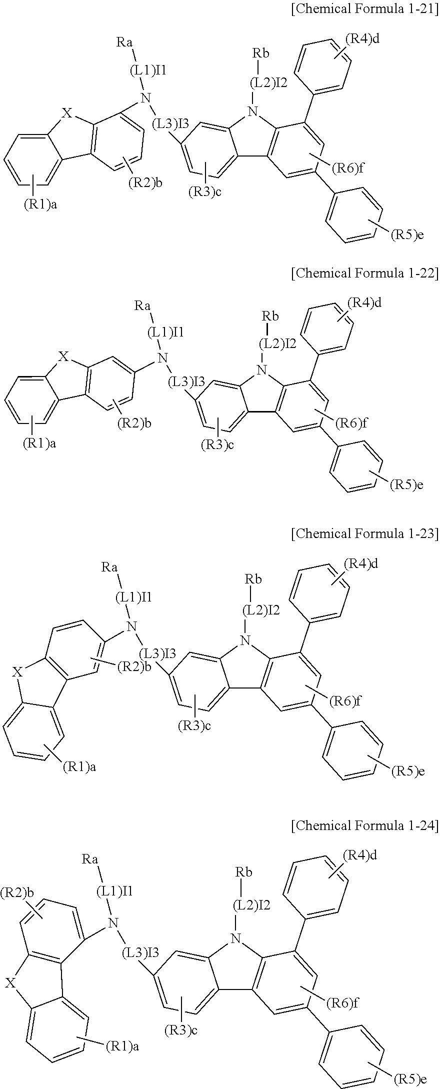

In Chemical Formulae 1-21 to 1-24, the description of each substituent is the same as that described above.

According to an exemplary embodiment of the present specification, Chemical Formula 1-3 may be represented by any one of the following Chemical Formulae 1-31 to 1-34.

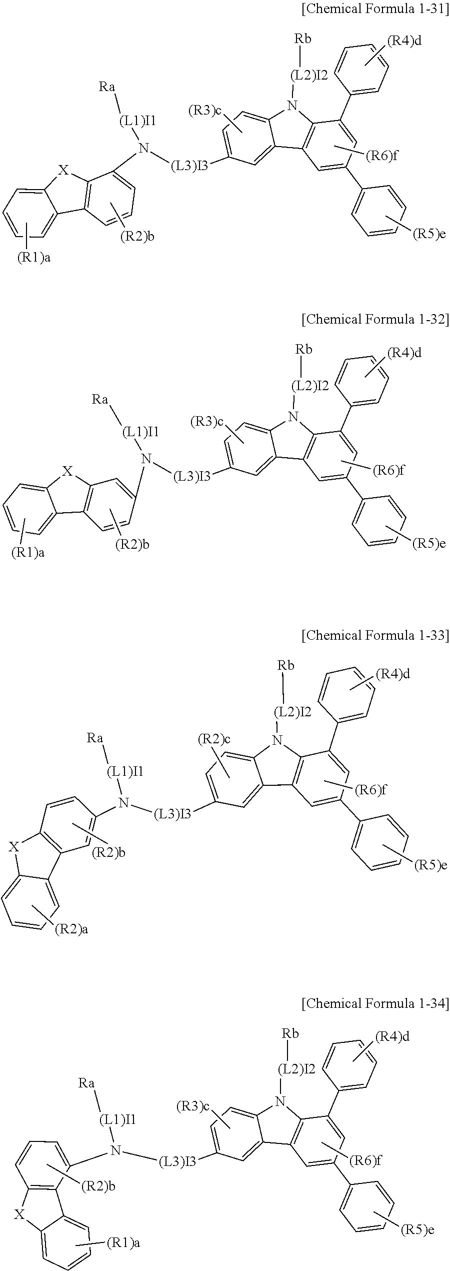

In Chemical Formulae 1-31 to 1-34, the description of each substituent is the same as that described above.

According to an exemplary embodiment of the present specification, Chemical Formula 1-4 may be represented by any one of the following Chemical Formulae 1-41 to 1-44.

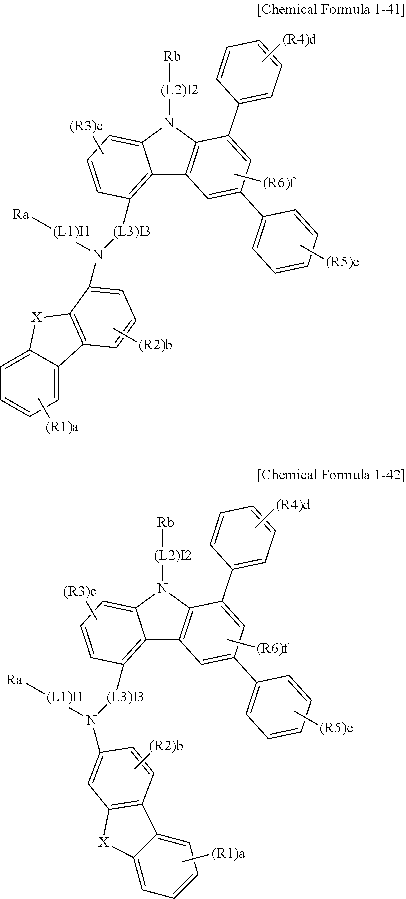

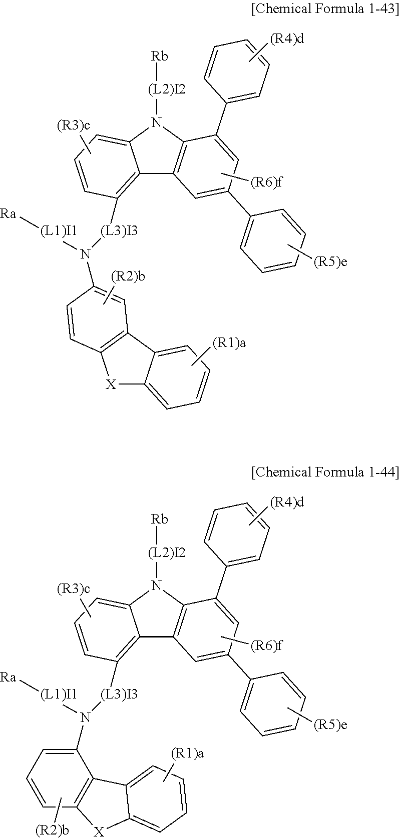

In Chemical Formulae 1-41 to 1-44, the description of each substituent is the same as that described above.

The heterocyclic compound according to exemplary embodiments may have low driving voltage, high light emitting efficiency, and/or long service life characteristics when used in an organic light emitting device.

According to an exemplary embodiment of the present specification, Ra and Rb are the same as or different from each other, and may be each independently a substituted or unsubstituted C6 to C40 aryl group; or a C2 to C40 heteroaryl group including at least one heteroatom from N, O, and S and substituted or unsubstituted.

According to an exemplary embodiment of the present specification, Ra and Rb are the same as or different from each other, and may be each independently a substituted or unsubstituted C6 to C30 aryl group; or a C2 to C30 heteroaryl group including at least one heteroatom from N, O, and S and substituted or unsubstituted.

According to an exemplary embodiment of the present specification, Ra and Rb are the same as or different from each other, and may be each independently a C6 to C30 aryl group substituted or unsubstituted with one or more substituents selected from the group consisting of deuterium and an alkyl group; or a C2 to C30 heteroaryl group including at least one heteroatom from N, O, and S and unsubstituted or substituted with one or more substituents selected from the group consisting of deuterium and an alkyl group.

According to an exemplary embodiment of the present specification, Ra and Rb are the same as or different from each other, and may be each independently a substituted or unsubstituted phenyl group; a substituted or unsubstituted biphenyl group; a substituted or unsubstituted terphenyl group; a substituted or unsubstituted naphthyl group; a substituted or unsubstituted phenanthrenyl group; a substituted or unsubstituted triphenylenyl group; a substituted or unsubstituted fluorenyl group; a substituted or unsubstituted spirobifluorenyl group; a substituted or unsubstituted dibenzofuranyl group; a substituted or unsubstituted naphthobenzofuranyl group; a substituted or unsubstituted dibenzothiophenyl group; a substituted or unsubstituted naphthobenzothiophenyl group; or a substituted or unsubstituted carbazolyl group.

According to an exemplary embodiment of the present specification, Ra and Rb are the same as or different from each other, and may be each independently a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a phenanthrenyl group unsubstituted or substituted with deuterium; a triphenylenyl group unsubstituted or substituted with deuterium; a fluorenyl group unsubstituted or substituted with one or more substituents selected from the group consisting of deuterium, an alkyl group, and an aryl group; a spirobifluorenyl group unsubstituted or substituted with deuterium; a dibenzofuranyl group unsubstituted or substituted with deuterium; a naphthobenzofuranyl group unsubstituted or substituted with deuterium; a dibenzothiophenyl group unsubstituted or substituted with deuterium; a naphthobenzothiophenyl group unsubstituted or substituted with deuterium; or a carbazolyl group unsubstituted or substituted with one or more substituents selected from the group consisting of deuterium, an aryl group, and a heteroaryl group.

According to an exemplary embodiment of the present specification, L1 to L3 are the same as or different from each other, and may be each independently a direct bond; or a substituted or unsubstituted C6 to C40 arylene group.

According to an exemplary embodiment of the present specification, L1 to L3 are the same as or different from each other, and may be each independently a direct bond; or a substituted or unsubstituted C6 to C30 arylene group.

According to an exemplary embodiment of the present specification, L1 to L3 are the same as or different from each other, and may be each independently a direct bond; or a C6 to C30 arylene group unsubstituted or substituted with deuterium.

According to an exemplary embodiment of the present specification, L1 to L3 are the same as or different from each other, and may be each independently a direct bond; or a substituted or unsubstituted phenylene group.

According to an exemplary embodiment of the present specification, L1 to L3 are the same as or different from each other, and may be each independently a direct bond; or a phenylene group unsubstituted or substituted with deuterium.

According to an exemplary embodiment of the present specification, L1 and L2 are the same as or different from each other, and may be each independently a direct bond; or a phenylene group unsubstituted or substituted with deuterium.

According to an exemplary embodiment of the present specification, L3 may be a direct bond.

According to an exemplary embodiment of the present specification, R1 to R6 are the same as or different from each other, and may be each independently hydrogen; deuterium; a cyano group; a halogen group; a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted silyl group; or a substituted or unsubstituted phosphine oxide group.

According to an exemplary embodiment of the present specification, R1 to R6 are the same as or different from each other, and may be each independently hydrogen; deuterium; a cyano group; a halogen group; a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted silyl group; or a substituted or unsubstituted phosphine oxide group.

According to an exemplary embodiment of the present specification, R1 to R6 are the same as or different from each other, and may be each independently hydrogen; deuterium; a cyano group; a halogen group; a C1 to C30 alkyl group unsubstituted or substituted with deuterium; a C3 to C30 cycloalkyl group unsubstituted or substituted with deuterium; a C2 to C30 heterocycloalkyl group unsubstituted or substituted with deuterium; a silyl group unsubstituted or substituted with one or more substituents selected from the group consisting of an alkyl group, an aryl group, and a heteroaryl group; or a phosphine oxide group unsubstituted or substituted with one or more substituents selected from the group consisting of an alkyl group, an aryl group, and a heteroaryl group.

According to an exemplary embodiment of the present specification, R1 to R6 are the same as or different from each other, and may be each independently hydrogen; deuterium; a halogen group; a C1 to C30 alkyl group unsubstituted or substituted with deuterium; a C3 to C30 cycloalkyl group unsubstituted or substituted with deuterium; or a C2 to C30 heterocycloalkyl group unsubstituted or substituted with deuterium.

According to an exemplary embodiment of the present specification, R1 to R6 are the same as or different from each other, and may be each independently hydrogen; or deuterium.

According to an exemplary embodiment of the present specification, the deuterium contents of the heterocyclic compound represented by Chemical Formula 1 are the same as or different from each other, and may be each independently 0% or 1% to 100%.

According to an exemplary embodiment of the present specification, the deuterium contents of the heterocyclic compound represented by Chemical Formula 1 are the same as or different from each other, and may be each independently 0% or 10% to 100%.

According to an exemplary embodiment of the present specification, the deuterium contents of the heterocyclic compound represented by Chemical Formula 1 are the same as or different from each other, and may be each independently 0% or 20% to 100%.

According to an exemplary embodiment of the present specification, the deuterium contents of the heterocyclic compound represented by Chemical Formula 1 are the same as or different from each other, and may be each independently 0% or 30% to 100%.

According to an exemplary embodiment of the present specification, the deuterium contents of the heterocyclic compound represented by Chemical Formula 1 are the same as or different from each other, and may be each independently 0% or 60% to 100%.

According to an exemplary embodiment of the present specification, the deuterium contents of the heterocyclic compound represented by Chemical Formula 1 are the same as or different from each other, and may be each independently 0% or 80% to 100%.

According to an exemplary embodiment of the present specification, the deuterium contents of the heterocyclic compound represented by Chemical Formula 1 are the same as or different from each other, and may be each independently 0% or 90% to 100%.

According to an exemplary embodiment of the present specification, Chemical Formula 1 may be represented by any one of the following compounds.

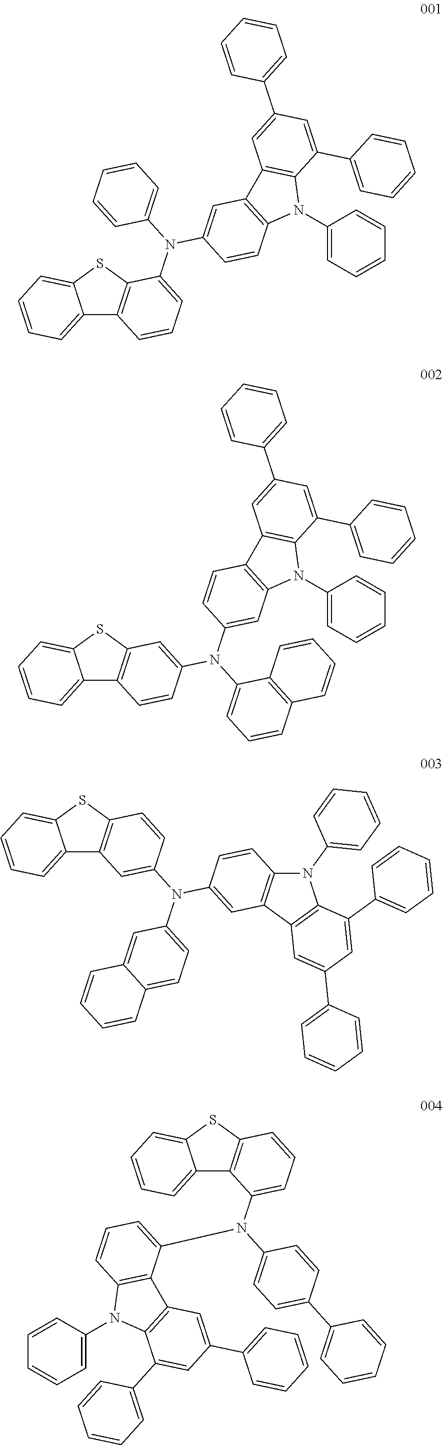 [SMILES: c1ccc(-c2cc(-c3ccccc3)c3c(c2)c2cc(N(c4ccccc4)c4cccc5c4sc4ccccc45)ccc2n3-c2ccccc2)cc1 ; c1ccc(-c2cc(-c3ccccc3)c3c(c2)c2ccc(N(c4ccc5c(c4)sc4ccccc45)c4cccc5ccccc45)cc2n3-c2ccccc2)cc1 ; c1ccc(-c2cc(-c3ccccc3)c3c(c2)c2cc(N(c4ccc5ccccc5c4)c4ccc5sc6ccccc6c5c4)ccc2n3-c2ccccc2)cc1 ; c1ccc(-c2ccc(N(c3cccc4sc5ccccc5c34)c3cccc4c3c3cc(-c5ccccc5)cc(-c5ccccc5)c3n4-c3ccccc3)cc2)cc1]

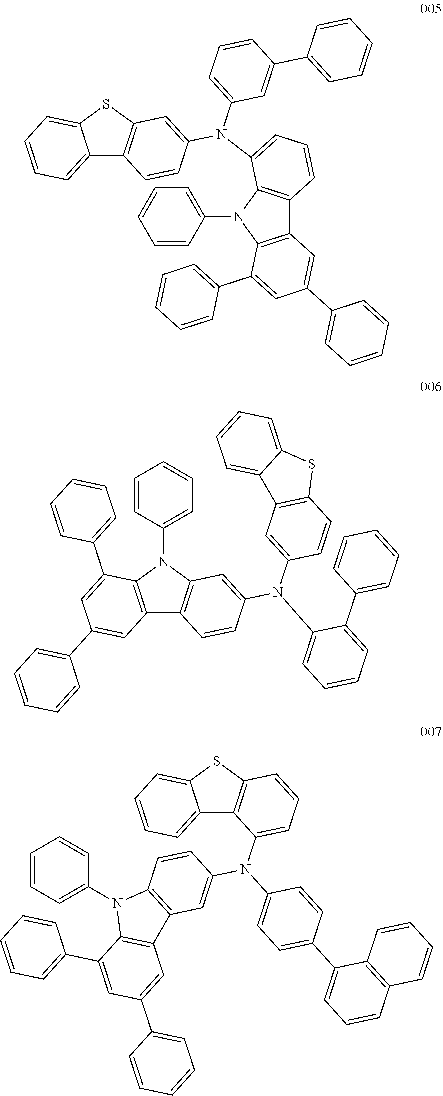 [SMILES: c1ccc(-c2cccc(N(c3ccc4c(c3)sc3ccccc34)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccc5sc6ccccc6c5c4)c4ccccc4-c4ccccc4)cc2n3-c2ccccc2)cc1 ; c1ccc(-c2cc(-c3ccccc3)c3c(c2)c2cc(N(c4ccc(-c5cccc6ccccc56)cc4)c4cccc5sc6ccccc6c45)ccc2n3-c2ccccc2)cc1]

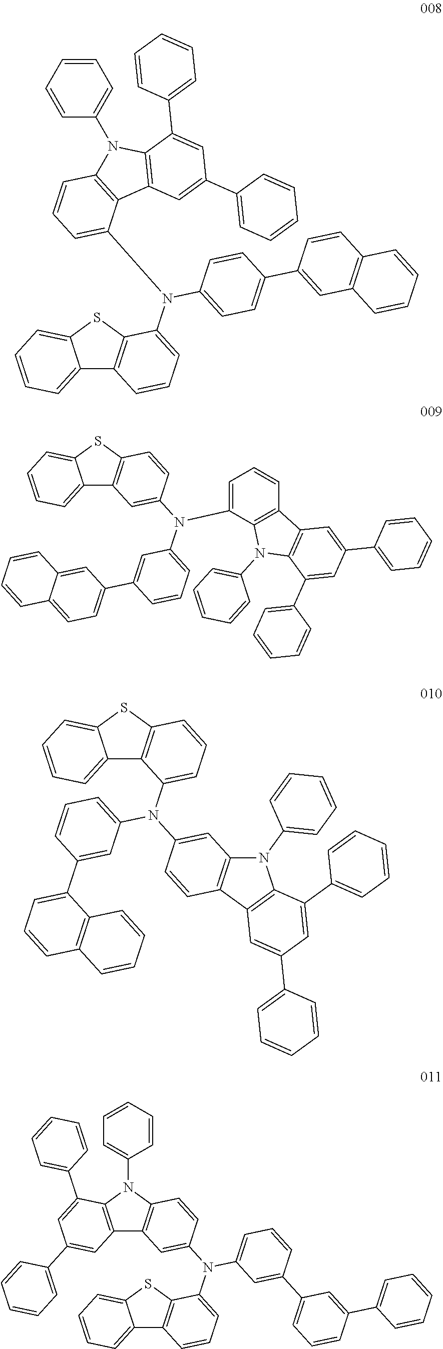 [SMILES: c1ccc(-c2cc(-c3ccccc3)c3c(c2)c2c(N(c4ccc(-c5ccc6ccccc6c5)cc4)c4cccc5c4sc4ccccc45)cccc2n3-c2ccccc2)cc1 ; c1ccc(-c2cc(-c3ccccc3)c3c(c2)c2cccc(N(c4cccc(-c5ccc6ccccc6c5)c4)c4ccc5sc6ccccc6c5c4)c2n3-c2ccccc2)cc1 ; c1ccc(-c2cc(-c3ccccc3)c3c(c2)c2ccc(N(c4cccc(-c5cccc6ccccc56)c4)c4cccc5sc6ccccc6c45)cc2n3-c2ccccc2)cc1 ; c1ccc(-c2cccc(-c3cccc(N(c4ccc5c(c4)c4cc(-c6ccccc6)cc(-c6ccccc6)c4n5-c4ccccc4)c4cccc5c4sc4ccccc45)c3)c2)cc1]

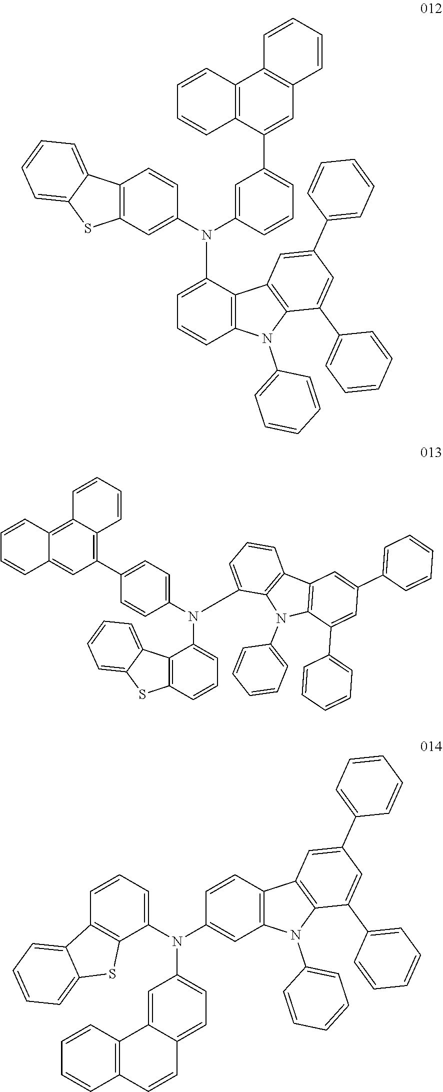 [SMILES: c1ccc(-c2cc(-c3ccccc3)c3c(c2)c2c(N(c4cccc(-c5cc6ccccc6c6ccccc56)c4)c4ccc5c(c4)sc4ccccc45)cccc2n3-c2ccccc2)cc1 ; c1ccc(-c2cc(-c3ccccc3)c3c(c2)c2cccc(N(c4ccc(-c5cc6ccccc6c6ccccc56)cc4)c4cccc5sc6ccccc6c45)c2n3-c2ccccc2)cc1 ; c1ccc(-c2cc(-c3ccccc3)c3c(c2)c2ccc(N(c4ccc5ccc6ccccc6c5c4)c4cccc5c4sc4ccccc45)cc2n3-c2ccccc2)cc1]

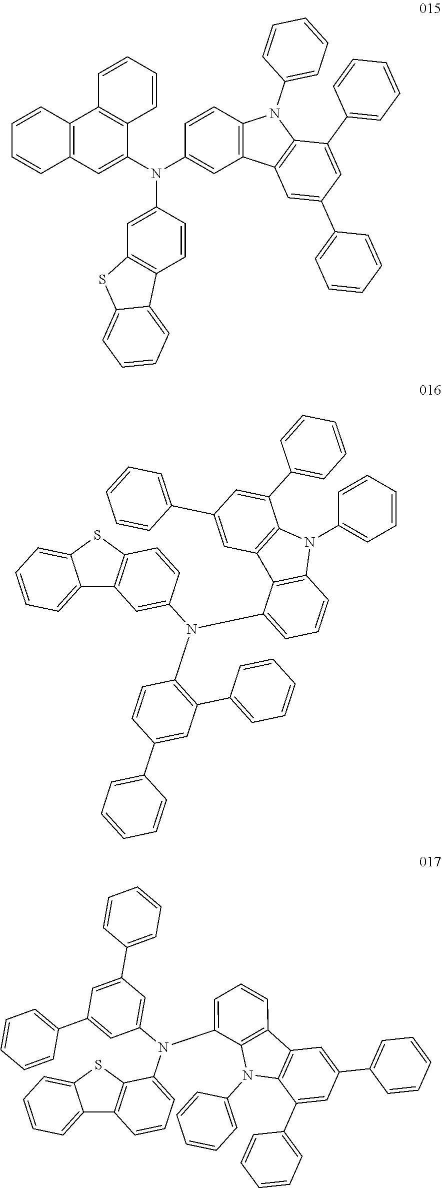 [SMILES: c1ccc(-c2cc(-c3ccccc3)c3c(c2)c2cc(N(c4ccc5c(c4)sc4ccccc45)c4cc5ccccc5c5ccccc45)ccc2n3-c2ccccc2)cc1 ; c1ccc(-c2ccc(N(c3ccc4sc5ccccc5c4c3)c3cccc4c3c3cc(-c5ccccc5)cc(-c5ccccc5)c3n4-c3ccccc3)c(-c3ccccc3)c2)cc1 ; c1ccc(-c2cc(-c3ccccc3)cc(N(c3cccc4c3sc3ccccc34)c3cccc4c5cc(-c6ccccc6)cc(-c6ccccc6)c5n(-c5ccccc5)c34)c2)cc1]

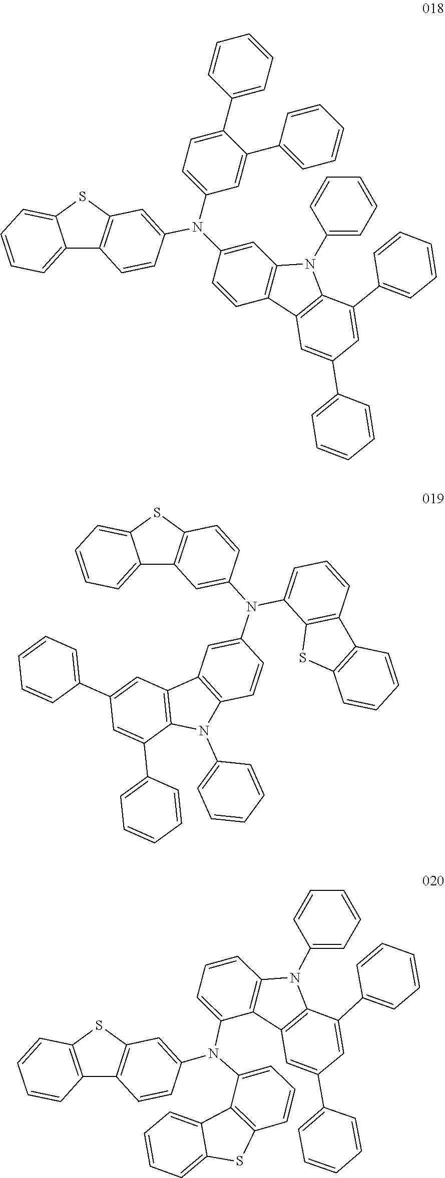 [SMILES: c1ccc(-c2cc(-c3ccccc3)c3c(c2)c2ccc(N(c4ccc(-c5ccccc5)c(-c5ccccc5)c4)c4ccc5c(c4)sc4ccccc45)cc2n3-c2ccccc2)cc1 ; c1ccc(-c2cc(-c3ccccc3)c3c(c2)c2cc(N(c4ccc5sc6ccccc6c5c4)c4cccc5c4sc4ccccc45)ccc2n3-c2ccccc2)cc1 ; c1ccc(-c2cc(-c3ccccc3)c3c(c2)c2c(N(c4ccc5c(c4)sc4ccccc45)c4cccc5sc6ccccc6c45)cccc2n3-c2ccccc2)cc1]

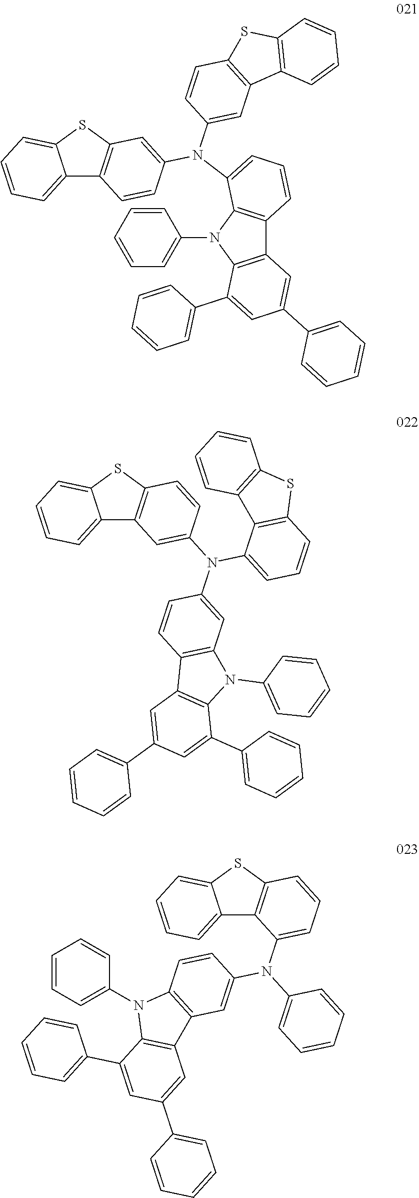 [SMILES: c1ccc(-c2cc(-c3ccccc3)c3c(c2)c2cccc(N(c4ccc5c(c4)sc4ccccc45)c4ccc5sc6ccccc6c5c4)c2n3-c2ccccc2)cc1 ; c1ccc(-c2cc(-c3ccccc3)c3c(c2)c2ccc(N(c4ccc5sc6ccccc6c5c4)c4cccc5sc6ccccc6c45)cc2n3-c2ccccc2)cc1 ; c1ccc(-c2cc(-c3ccccc3)c3c(c2)c2cc(N(c4ccccc4)c4cccc5sc6ccccc6c45)ccc2n3-c2ccccc2)cc1]

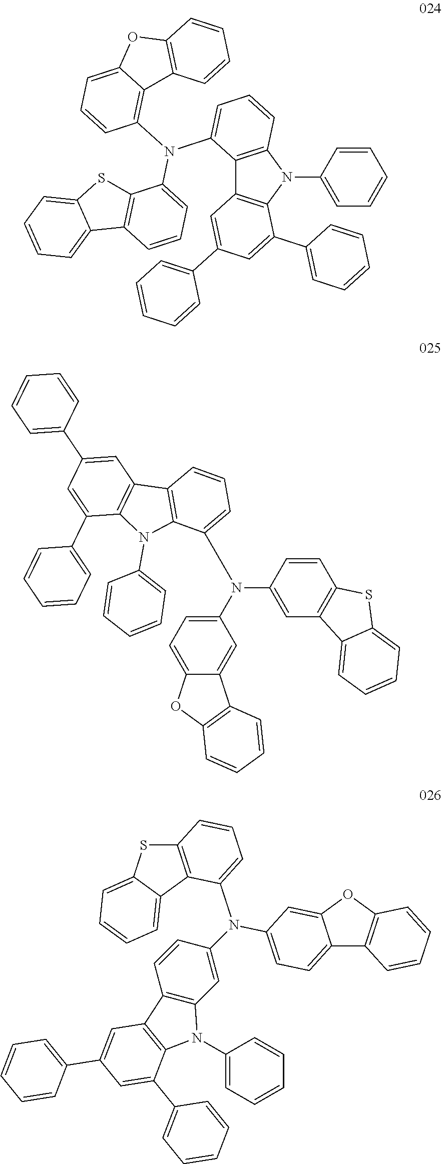 [SMILES: c1ccc(-c2cc(-c3ccccc3)c3c(c2)c2c(N(c4cccc5c4sc4ccccc45)c4cccc5oc6ccccc6c45)cccc2n3-c2ccccc2)cc1 ; c1ccc(-c2cc(-c3ccccc3)c3c(c2)c2cccc(N(c4ccc5oc6ccccc6c5c4)c4ccc5sc6ccccc6c5c4)c2n3-c2ccccc2)cc1 ; c1ccc(-c2cc(-c3ccccc3)c3c(c2)c2ccc(N(c4ccc5c(c4)oc4ccccc45)c4cccc5sc6ccccc6c45)cc2n3-c2ccccc2)cc1]

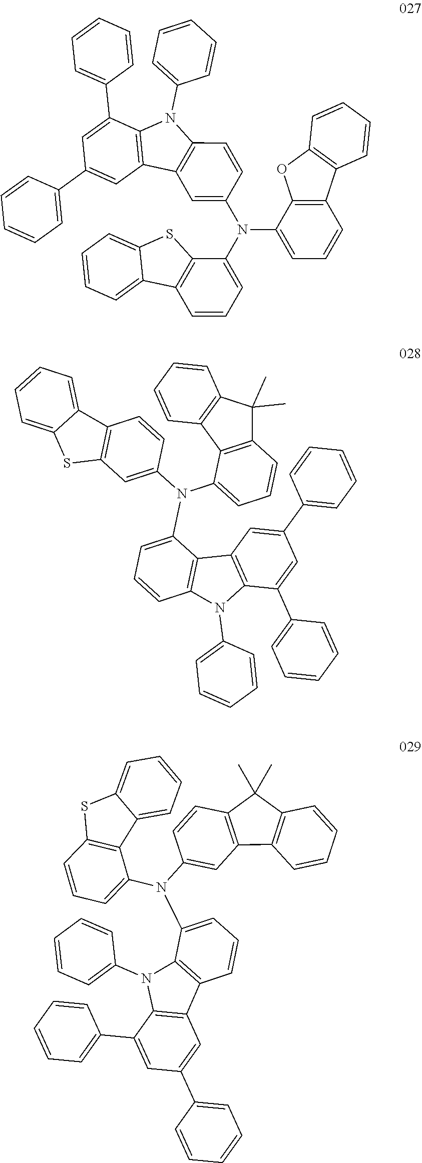 [SMILES: c1ccc(-c2cc(-c3ccccc3)c3c(c2)c2cc(N(c4cccc5c4oc4ccccc45)c4cccc5c4sc4ccccc45)ccc2n3-c2ccccc2)cc1 ; CC1(C)c2ccccc2-c2c(N(c3ccc4c(c3)sc3ccccc34)c3cccc4c3c3cc(-c5ccccc5)cc(-c5ccccc5)c3n4-c3ccccc3)cccc21 ; CC1(C)c2ccccc2-c2cc(N(c3cccc4sc5ccccc5c34)c3cccc4c5cc(-c6ccccc6)cc(-c6ccccc6)c5n(-c5ccccc5)c34)ccc21]

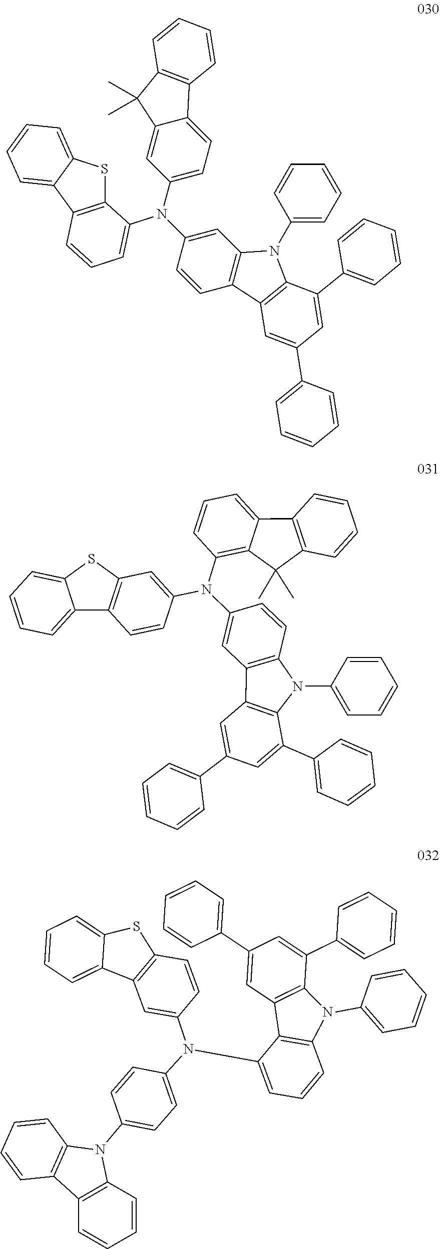 [SMILES: CC1(C)c2ccccc2-c2ccc(N(c3ccc4c5cc(-c6ccccc6)cc(-c6ccccc6)c5n(-c5ccccc5)c4c3)c3cccc4c3sc3ccccc34)cc21 ; CC1(C)c2ccccc2-c2cccc(N(c3ccc4c(c3)sc3ccccc34)c3ccc4c(c3)c3cc(-c5ccccc5)cc(-c5ccccc5)c3n4-c3ccccc3)c21 ; c1ccc(-c2cc(-c3ccccc3)c3c(c2)c2c(N(c4ccc(-n5c6ccccc6c6ccccc65)cc4)c4ccc5sc6ccccc6c5c4)cccc2n3-c2ccccc2)cc1]

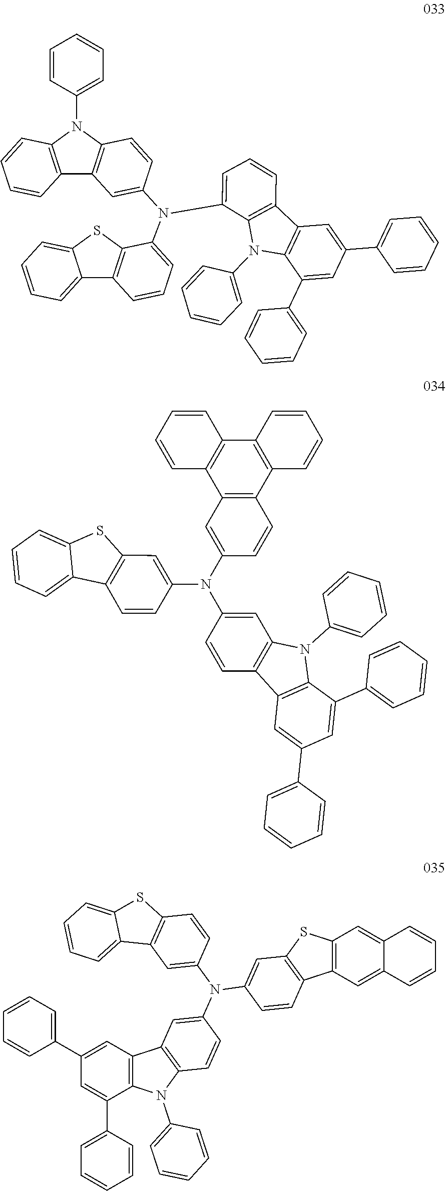 [SMILES: c1ccc(-c2cc(-c3ccccc3)c3c(c2)c2cccc(N(c4ccc5c(c4)c4ccccc4n5-c4ccccc4)c4cccc5c4sc4ccccc45)c2n3-c2ccccc2)cc1 ; c1ccc(-c2cc(-c3ccccc3)c3c(c2)c2ccc(N(c4ccc5c(c4)sc4ccccc45)c4ccc5c6ccccc6c6ccccc6c5c4)cc2n3-c2ccccc2)cc1 ; c1ccc(-c2cc(-c3ccccc3)c3c(c2)c2cc(N(c4ccc5c(c4)sc4cc6ccccc6cc45)c4ccc5sc6ccccc6c5c4)ccc2n3-c2ccccc2)cc1]

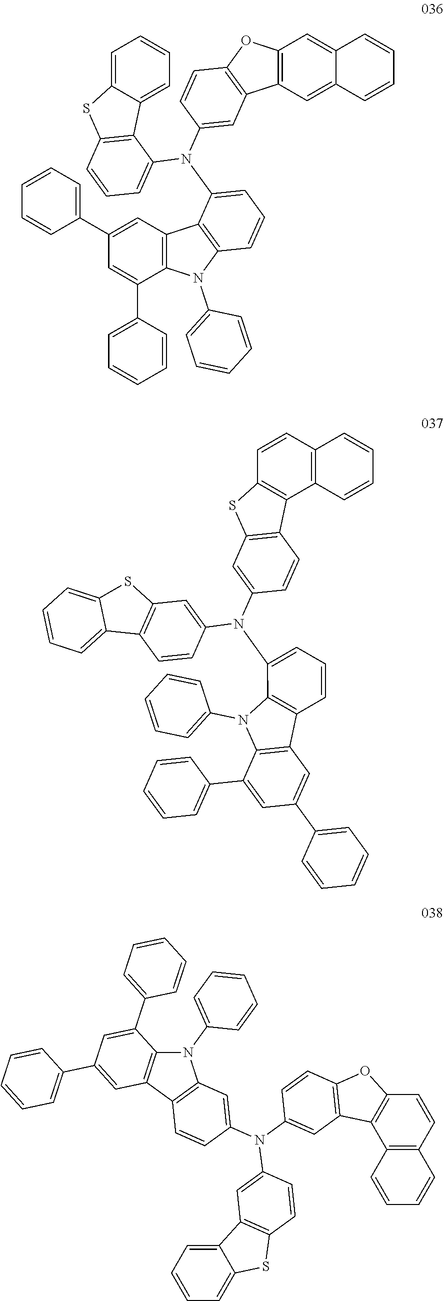 [SMILES: c1ccc(-c2cc(-c3ccccc3)c3c(c2)c2c(N(c4ccc5oc6cc7ccccc7cc6c5c4)c4cccc5sc6ccccc6c45)cccc2n3-c2ccccc2)cc1 ; c1ccc(-c2cc(-c3ccccc3)c3c(c2)c2cccc(N(c4ccc5c(c4)sc4ccccc45)c4ccc5c(c4)sc4ccc6ccccc6c45)c2n3-c2ccccc2)cc1 ; c1ccc(-c2cc(-c3ccccc3)c3c(c2)c2ccc(N(c4ccc5sc6ccccc6c5c4)c4ccc5oc6ccc7ccccc7c6c5c4)cc2n3-c2ccccc2)cc1]

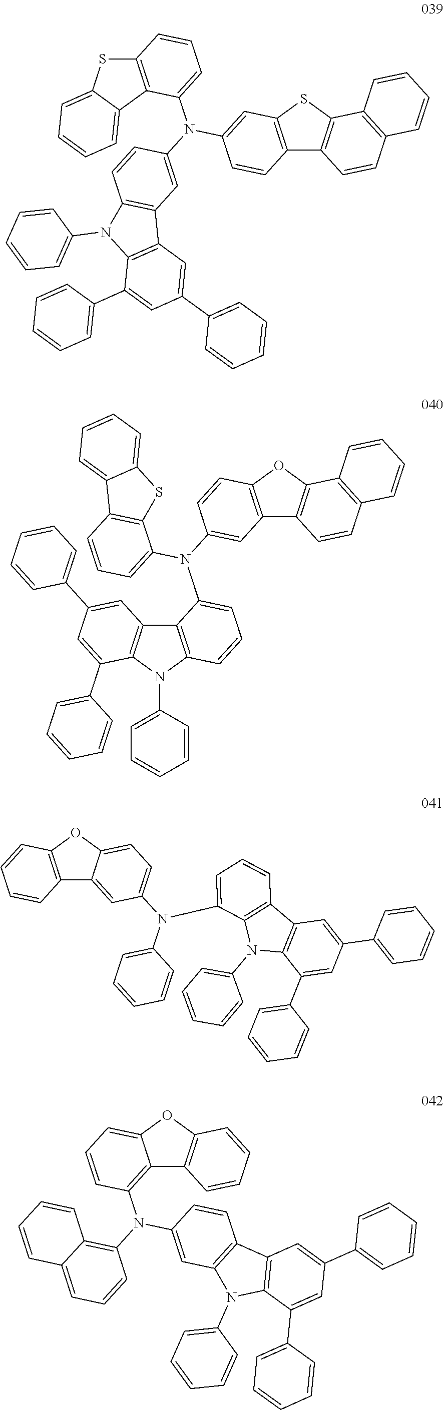 [SMILES: c1ccc(-c2cc(-c3ccccc3)c3c(c2)c2cc(N(c4ccc5c(c4)sc4c6ccccc6ccc54)c4cccc5sc6ccccc6c45)ccc2n3-c2ccccc2)cc1 ; c1ccc(-c2cc(-c3ccccc3)c3c(c2)c2c(N(c4ccc5oc6c7ccccc7ccc6c5c4)c4cccc5c4sc4ccccc45)cccc2n3-c2ccccc2)cc1 ; c1ccc(-c2cc(-c3ccccc3)c3c(c2)c2cccc(N(c4ccccc4)c4ccc5oc6ccccc6c5c4)c2n3-c2ccccc2)cc1 ; c1ccc(-c2cc(-c3ccccc3)c3c(c2)c2ccc(N(c4cccc5ccccc45)c4cccc5oc6ccccc6c45)cc2n3-c2ccccc2)cc1]

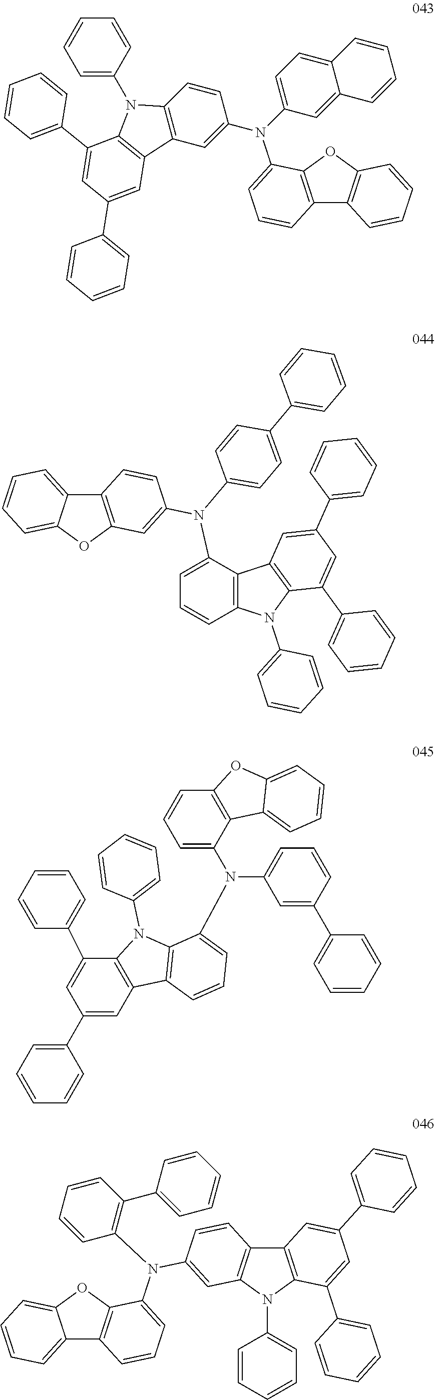 [SMILES: c1ccc(-c2cc(-c3ccccc3)c3c(c2)c2cc(N(c4ccc5ccccc5c4)c4cccc5c4oc4ccccc45)ccc2n3-c2ccccc2)cc1 ; c1ccc(-c2ccc(N(c3ccc4c(c3)oc3ccccc34)c3cccc4c3c3cc(-c5ccccc5)cc(-c5ccccc5)c3n4-c3ccccc3)cc2)cc1 ; c1ccc(-c2cccc(N(c3cccc4oc5ccccc5c34)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccccc4-c4ccccc4)c4cccc5c4oc4ccccc45)cc2n3-c2ccccc2)cc1]

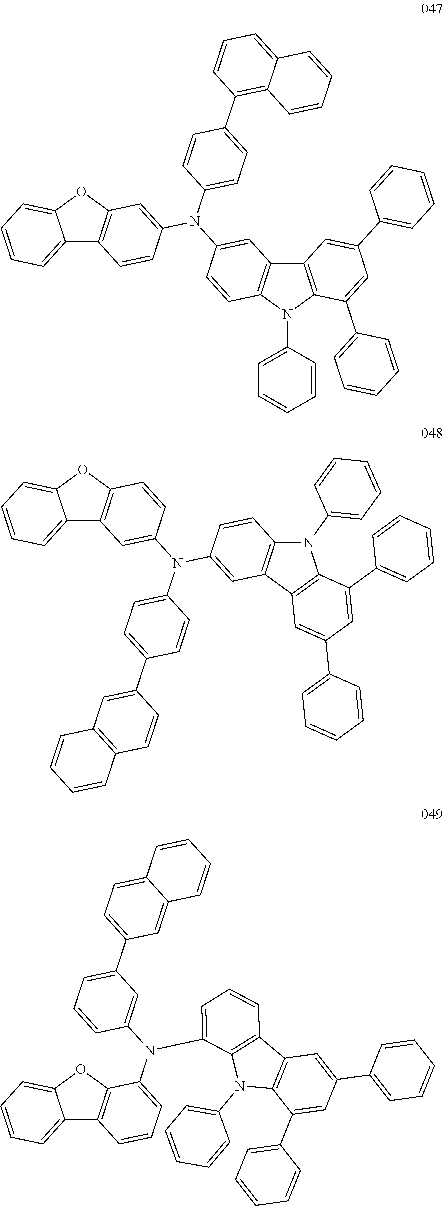 [SMILES: c1ccc(-c2cc(-c3ccccc3)c3c(c2)c2cc(N(c4ccc(-c5cccc6ccccc56)cc4)c4ccc5c(c4)oc4ccccc45)ccc2n3-c2ccccc2)cc1 ; c1ccc(-c2cc(-c3ccccc3)c3c(c2)c2cc(N(c4ccc(-c5ccc6ccccc6c5)cc4)c4ccc5oc6ccccc6c5c4)ccc2n3-c2ccccc2)cc1 ; c1ccc(-c2cc(-c3ccccc3)c3c(c2)c2cccc(N(c4cccc(-c5ccc6ccccc6c5)c4)c4cccc5c4oc4ccccc45)c2n3-c2ccccc2)cc1]

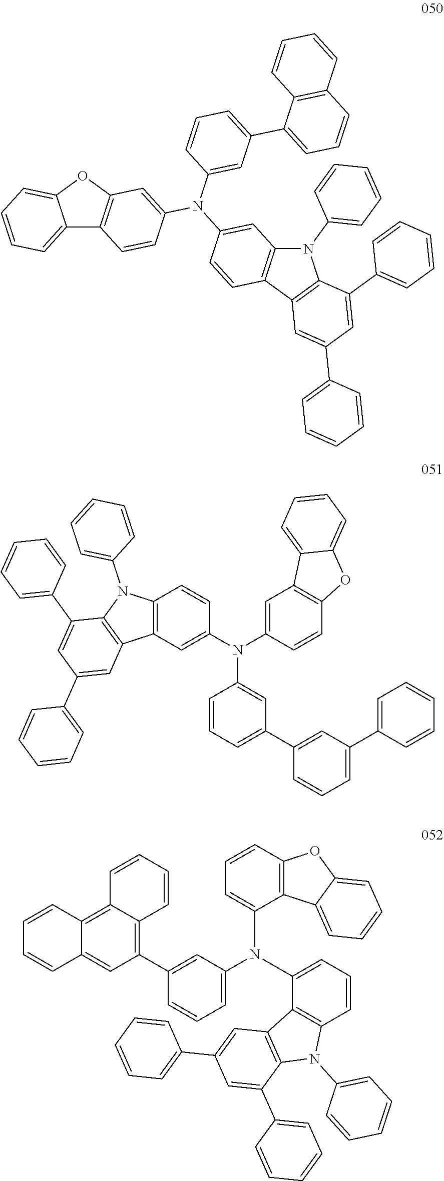 [SMILES: c1ccc(-c2cc(-c3ccccc3)c3c(c2)c2ccc(N(c4cccc(-c5cccc6ccccc56)c4)c4ccc5c(c4)oc4ccccc45)cc2n3-c2ccccc2)cc1 ; c1ccc(-c2cccc(-c3cccc(N(c4ccc5oc6ccccc6c5c4)c4ccc5c(c4)c4cc(-c6ccccc6)cc(-c6ccccc6)c4n5-c4ccccc4)c3)c2)cc1 ; c1ccc(-c2cc(-c3ccccc3)c3c(c2)c2c(N(c4cccc(-c5cc6ccccc6c6ccccc56)c4)c4cccc5oc6ccccc6c45)cccc2n3-c2ccccc2)cc1]

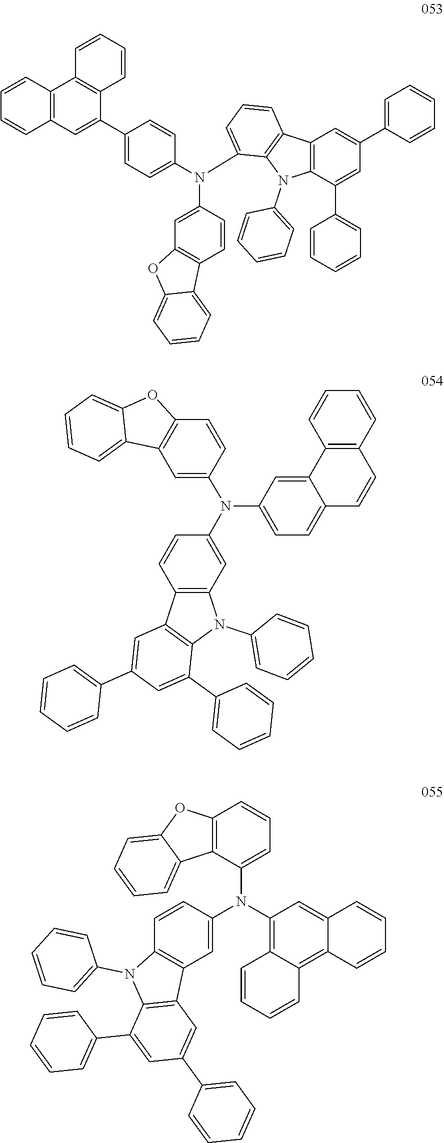 [SMILES: c1ccc(-c2cc(-c3ccccc3)c3c(c2)c2cccc(N(c4ccc(-c5cc6ccccc6c6ccccc56)cc4)c4ccc5c(c4)oc4ccccc45)c2n3-c2ccccc2)cc1 ; c1ccc(-c2cc(-c3ccccc3)c3c(c2)c2ccc(N(c4ccc5ccc6ccccc6c5c4)c4ccc5oc6ccccc6c5c4)cc2n3-c2ccccc2)cc1 ; c1ccc(-c2cc(-c3ccccc3)c3c(c2)c2cc(N(c4cc5ccccc5c5ccccc45)c4cccc5oc6ccccc6c45)ccc2n3-c2ccccc2)cc1]

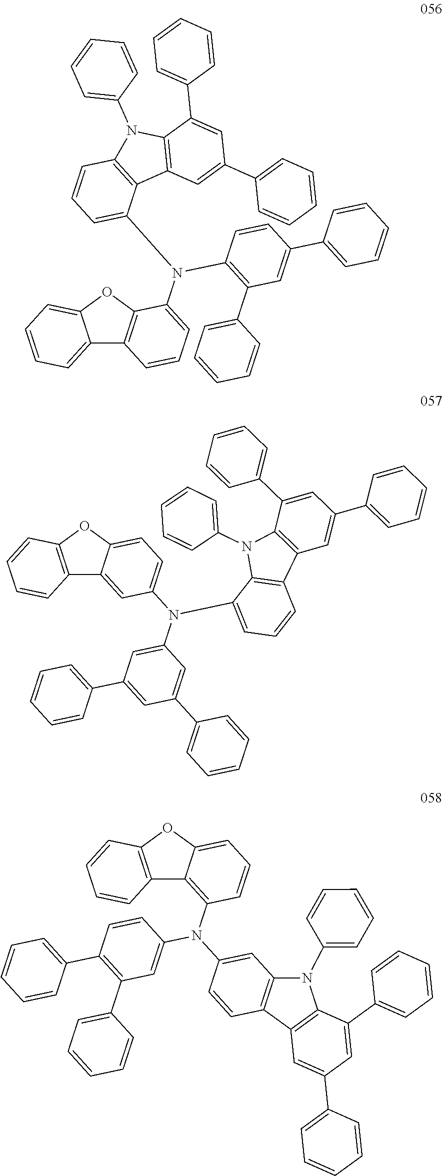 [SMILES: c1ccc(-c2ccc(N(c3cccc4c3oc3ccccc34)c3cccc4c3c3cc(-c5ccccc5)cc(-c5ccccc5)c3n4-c3ccccc3)c(-c3ccccc3)c2)cc1 ; c1ccc(-c2cc(-c3ccccc3)cc(N(c3ccc4oc5ccccc5c4c3)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccc(-c5ccccc5)c(-c5ccccc5)c4)c4cccc5oc6ccccc6c45)cc2n3-c2ccccc2)cc1]

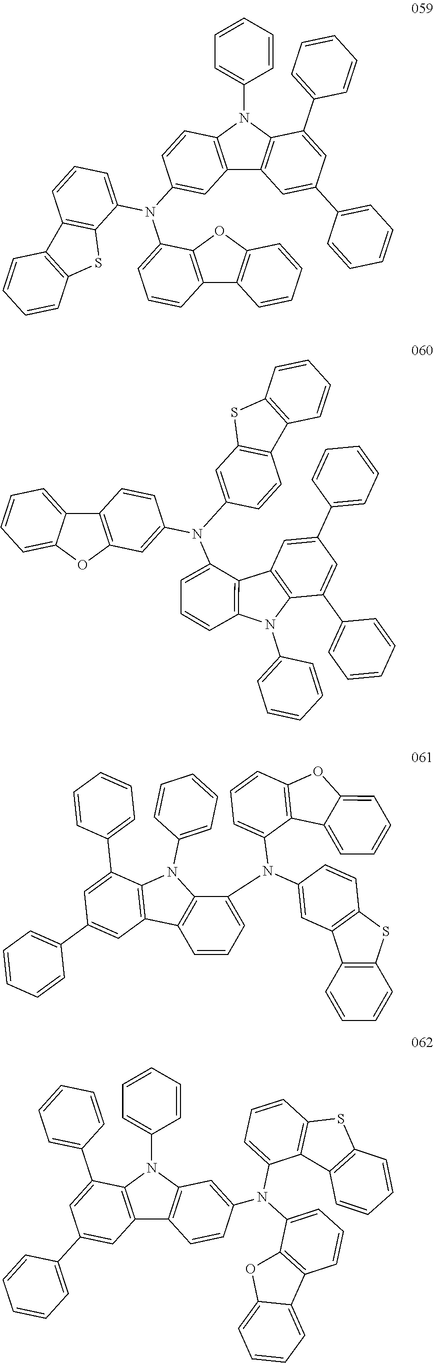 [SMILES: c1ccc(-c2cc(-c3ccccc3)c3c(c2)c2cc(N(c4cccc5c4oc4ccccc45)c4cccc5c4sc4ccccc45)ccc2n3-c2ccccc2)cc1 ; c1ccc(-c2cc(-c3ccccc3)c3c(c2)c2c(N(c4ccc5c(c4)oc4ccccc45)c4ccc5c(c4)sc4ccccc45)cccc2n3-c2ccccc2)cc1 ; c1ccc(-c2cc(-c3ccccc3)c3c(c2)c2cccc(N(c4ccc5sc6ccccc6c5c4)c4cccc5oc6ccccc6c45)c2n3-c2ccccc2)cc1 ; c1ccc(-c2cc(-c3ccccc3)c3c(c2)c2ccc(N(c4cccc5c4oc4ccccc45)c4cccc5sc6ccccc6c45)cc2n3-c2ccccc2)cc1]

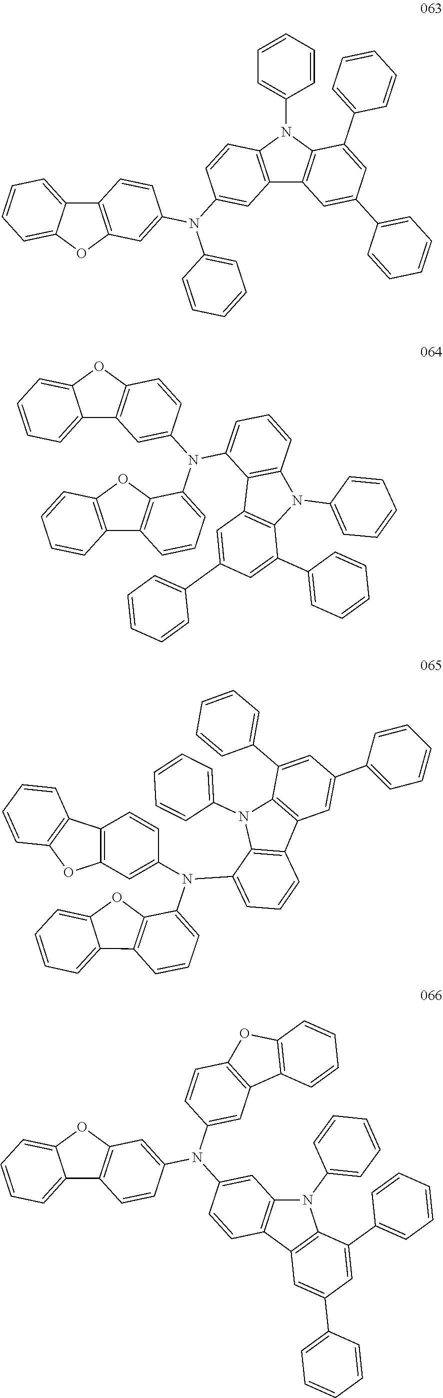 [SMILES: c1ccc(-c2cc(-c3ccccc3)c3c(c2)c2cc(N(c4ccccc4)c4ccc5c(c4)oc4ccccc45)ccc2n3-c2ccccc2)cc1 ; c1ccc(-c2cc(-c3ccccc3)c3c(c2)c2c(N(c4ccc5oc6ccccc6c5c4)c4cccc5c4oc4ccccc45)cccc2n3-c2ccccc2)cc1 ; c1ccc(-c2cc(-c3ccccc3)c3c(c2)c2cccc(N(c4ccc5c(c4)oc4ccccc45)c4cccc5c4oc4ccccc45)c2n3-c2ccccc2)cc1 ; c1ccc(-c2cc(-c3ccccc3)c3c(c2)c2ccc(N(c4ccc5c(c4)oc4ccccc45)c4ccc5oc6ccccc6c5c4)cc2n3-c2ccccc2)cc1]

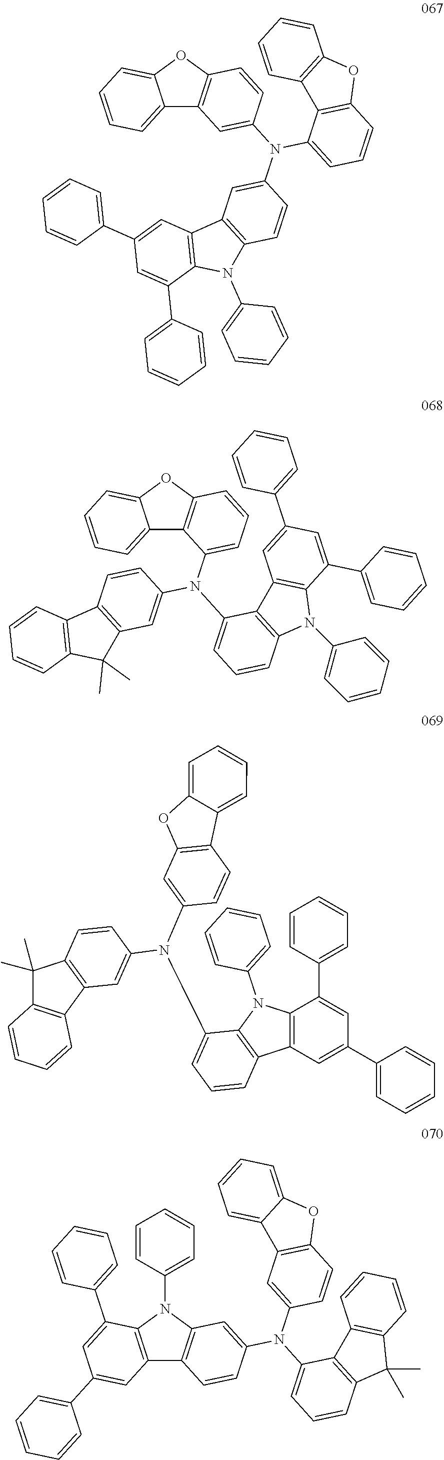 [SMILES: c1ccc(-c2cc(-c3ccccc3)c3c(c2)c2cc(N(c4ccc5oc6ccccc6c5c4)c4cccc5oc6ccccc6c45)ccc2n3-c2ccccc2)cc1 ; CC1(C)c2ccccc2-c2ccc(N(c3cccc4oc5ccccc5c34)c3cccc4c3c3cc(-c5ccccc5)cc(-c5ccccc5)c3n4-c3ccccc3)cc21 ; CC1(C)c2ccccc2-c2cc(N(c3ccc4c(c3)oc3ccccc34)c3cccc4c5cc(-c6ccccc6)cc(-c6ccccc6)c5n(-c5ccccc5)c34)ccc21 ; CC1(C)c2ccccc2-c2c(N(c3ccc4oc5ccccc5c4c3)c3ccc4c5cc(-c6ccccc6)cc(-c6ccccc6)c5n(-c5ccccc5)c4c3)cccc21]

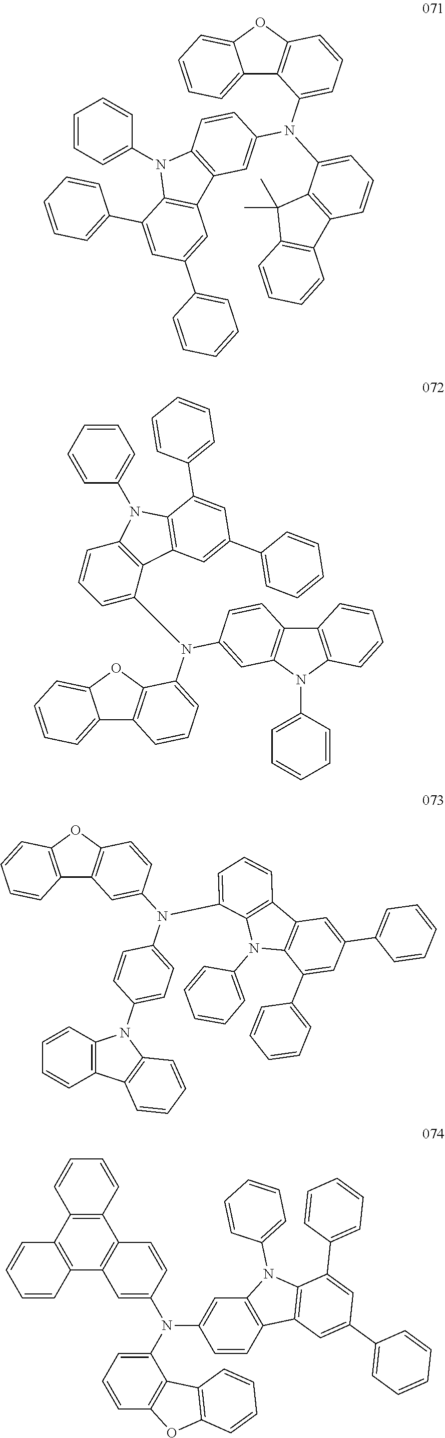 [SMILES: CC1(C)c2ccccc2-c2cccc(N(c3ccc4c(c3)c3cc(-c5ccccc5)cc(-c5ccccc5)c3n4-c3ccccc3)c3cccc4oc5ccccc5c34)c21 ; c1ccc(-c2cc(-c3ccccc3)c3c(c2)c2c(N(c4ccc5c6ccccc6n(-c6ccccc6)c5c4)c4cccc5c4oc4ccccc45)cccc2n3-c2ccccc2)cc1 ; c1ccc(-c2cc(-c3ccccc3)c3c(c2)c2cccc(N(c4ccc(-n5c6ccccc6c6ccccc65)cc4)c4ccc5oc6ccccc6c5c4)c2n3-c2ccccc2)cc1 ; c1ccc(-c2cc(-c3ccccc3)c3c(c2)c2ccc(N(c4ccc5c6ccccc6c6ccccc6c5c4)c4cccc5oc6ccccc6c45)cc2n3-c2ccccc2)cc1]

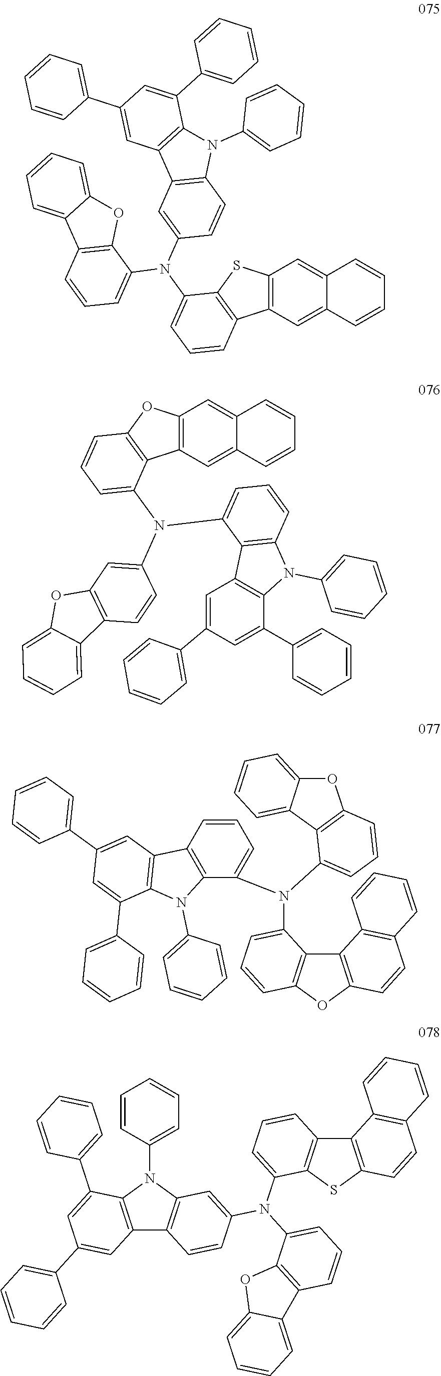 [SMILES: c1ccc(-c2cc(-c3ccccc3)c3c(c2)c2cc(N(c4cccc5c4oc4ccccc45)c4cccc5c4sc4cc6ccccc6cc45)ccc2n3-c2ccccc2)cc1 ; c1ccc(-c2cc(-c3ccccc3)c3c(c2)c2c(N(c4ccc5c(c4)oc4ccccc45)c4cccc5oc6cc7ccccc7cc6c45)cccc2n3-c2ccccc2)cc1 ; c1ccc(-c2cc(-c3ccccc3)c3c(c2)c2cccc(N(c4cccc5oc6ccccc6c45)c4cccc5oc6ccc7ccccc7c6c45)c2n3-c2ccccc2)cc1 ; c1ccc(-c2cc(-c3ccccc3)c3c(c2)c2ccc(N(c4cccc5c4oc4ccccc45)c4cccc5c4sc4ccc6ccccc6c45)cc2n3-c2ccccc2)cc1]

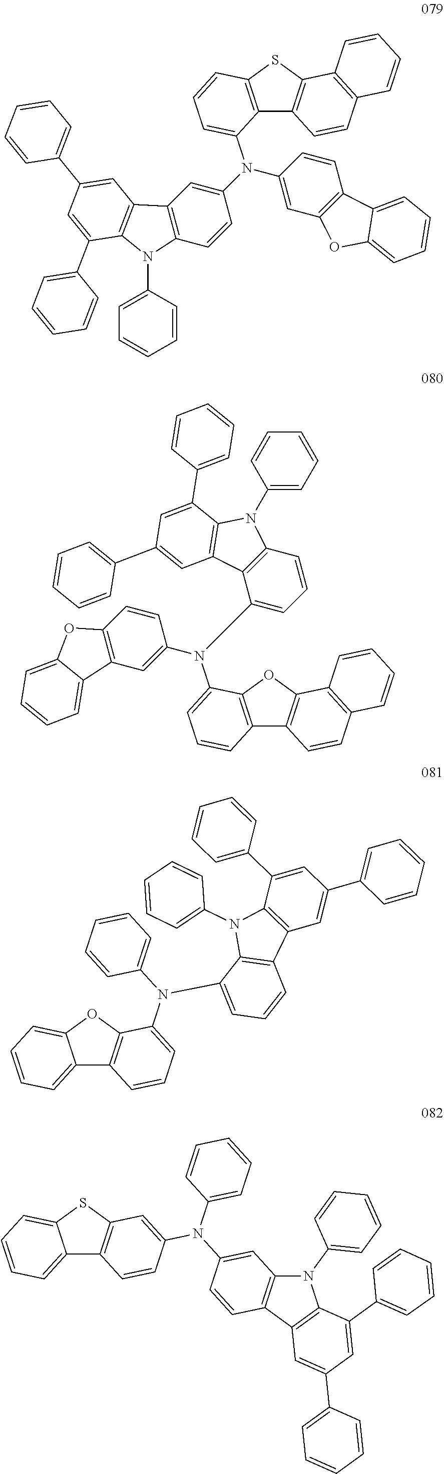

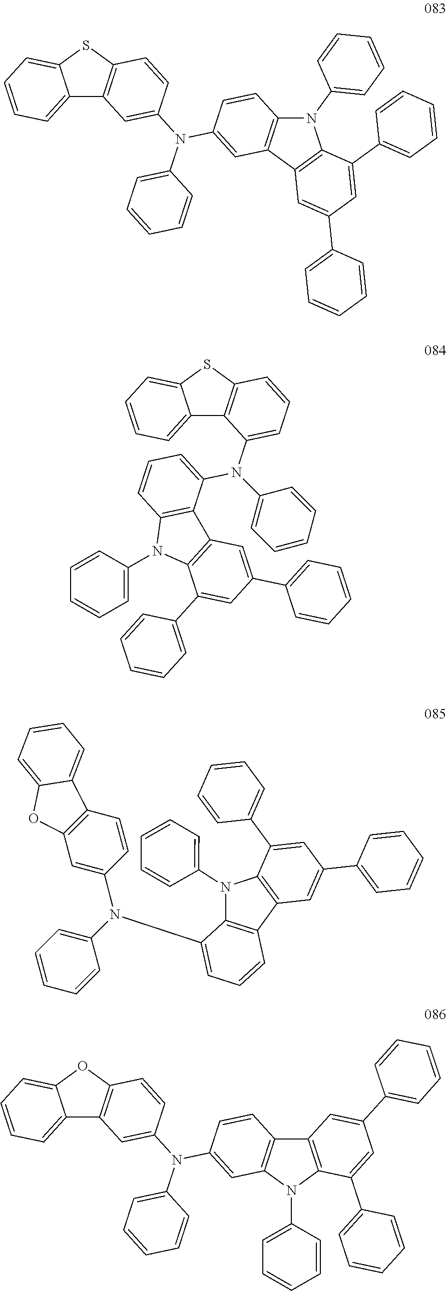 [SMILES: c1ccc(-c2cc(-c3ccccc3)c3c(c2)c2cc(N(c4ccccc4)c4ccc5sc6ccccc6c5c4)ccc2n3-c2ccccc2)cc1 ; c1ccc(-c2cc(-c3ccccc3)c3c(c2)c2c(N(c4ccccc4)c4cccc5sc6ccccc6c45)cccc2n3-c2ccccc2)cc1 ; c1ccc(-c2cc(-c3ccccc3)c3c(c2)c2cccc(N(c4ccccc4)c4ccc5c(c4)oc4ccccc45)c2n3-c2ccccc2)cc1 ; c1ccc(-c2cc(-c3ccccc3)c3c(c2)c2ccc(N(c4ccccc4)c4ccc5oc6ccccc6c5c4)cc2n3-c2ccccc2)cc1]

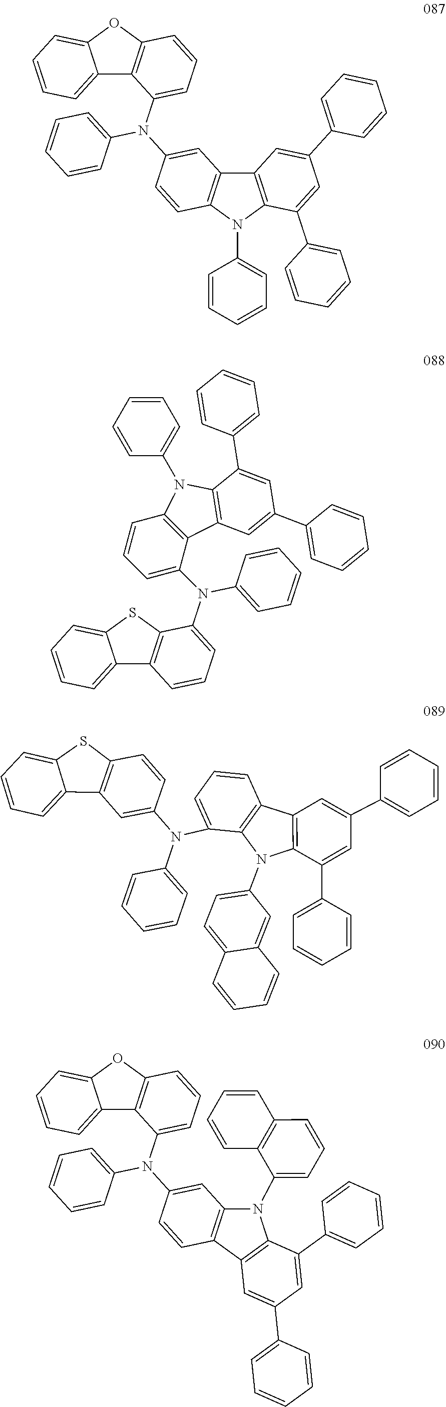 [SMILES: c1ccc(-c2cc(-c3ccccc3)c3c(c2)c2cc(N(c4ccccc4)c4cccc5oc6ccccc6c45)ccc2n3-c2ccccc2)cc1 ; c1ccc(-c2cc(-c3ccccc3)c3c(c2)c2c(N(c4ccccc4)c4cccc5c4sc4ccccc45)cccc2n3-c2ccccc2)cc1 ; c1ccc(-c2cc(-c3ccccc3)c3c(c2)c2cccc(N(c4ccccc4)c4ccc5sc6ccccc6c5c4)c2n3-c2ccc3ccccc3c2)cc1 ; c1ccc(-c2cc(-c3ccccc3)c3c(c2)c2ccc(N(c4ccccc4)c4cccc5oc6ccccc6c45)cc2n3-c2cccc3ccccc23)cc1]

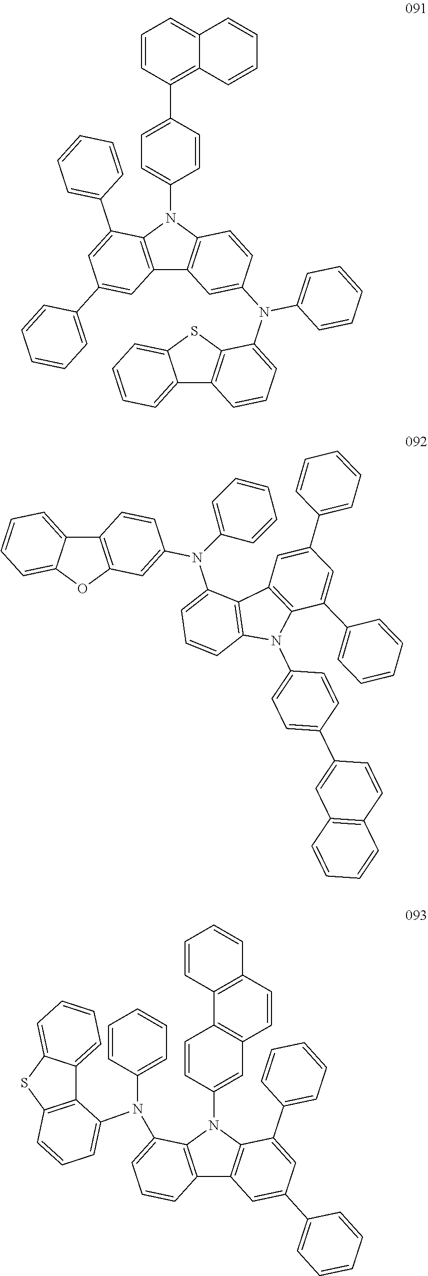 [SMILES: c1ccc(-c2cc(-c3ccccc3)c3c(c2)c2cc(N(c4ccccc4)c4cccc5c4sc4ccccc45)ccc2n3-c2ccc(-c3cccc4ccccc34)cc2)cc1 ; c1ccc(-c2cc(-c3ccccc3)c3c(c2)c2c(N(c4ccccc4)c4ccc5c(c4)oc4ccccc45)cccc2n3-c2ccc(-c3ccc4ccccc4c3)cc2)cc1 ; c1ccc(-c2cc(-c3ccccc3)c3c(c2)c2cccc(N(c4ccccc4)c4cccc5sc6ccccc6c45)c2n3-c2ccc3c(ccc4ccccc43)c2)cc1]

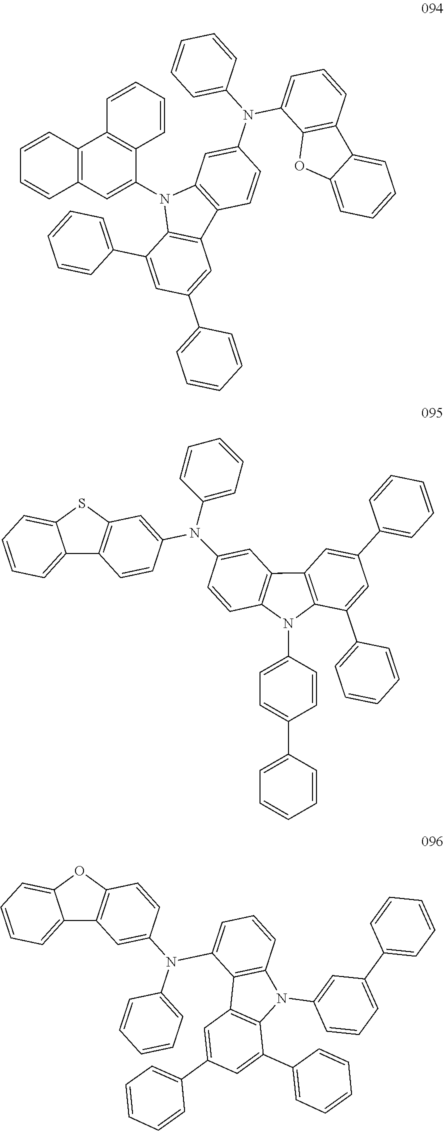 [SMILES: c1ccc(-c2cc(-c3ccccc3)c3c(c2)c2ccc(N(c4ccccc4)c4cccc5c4oc4ccccc45)cc2n3-c2cc3ccccc3c3ccccc23)cc1 ; c1ccc(-c2ccc(-n3c4ccc(N(c5ccccc5)c5ccc6c(c5)sc5ccccc56)cc4c4cc(-c5ccccc5)cc(-c5ccccc5)c43)cc2)cc1 ; c1ccc(-c2cccc(-n3c4cccc(N(c5ccccc5)c5ccc6oc7ccccc7c6c5)c4c4cc(-c5ccccc5)cc(-c5ccccc5)c43)c2)cc1]

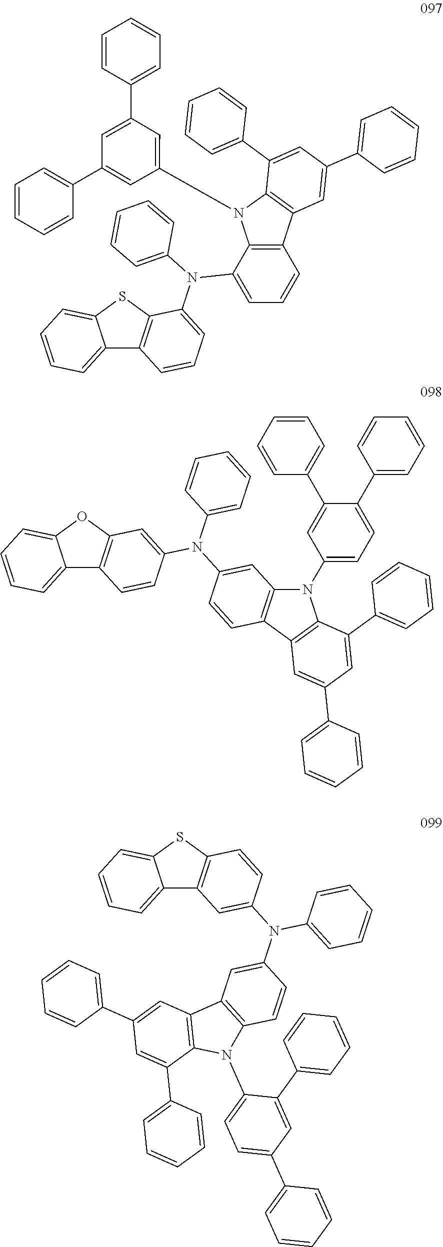 [SMILES: c1ccc(-c2cc(-c3ccccc3)cc(-n3c4c(-c5ccccc5)cc(-c5ccccc5)cc4c4cccc(N(c5ccccc5)c5cccc6c5sc5ccccc56)c43)c2)cc1 ; c1ccc(-c2cc(-c3ccccc3)c3c(c2)c2ccc(N(c4ccccc4)c4ccc5c(c4)oc4ccccc45)cc2n3-c2ccc(-c3ccccc3)c(-c3ccccc3)c2)cc1 ; c1ccc(-c2ccc(-n3c4ccc(N(c5ccccc5)c5ccc6sc7ccccc7c6c5)cc4c4cc(-c5ccccc5)cc(-c5ccccc5)c43)c(-c3ccccc3)c2)cc1]

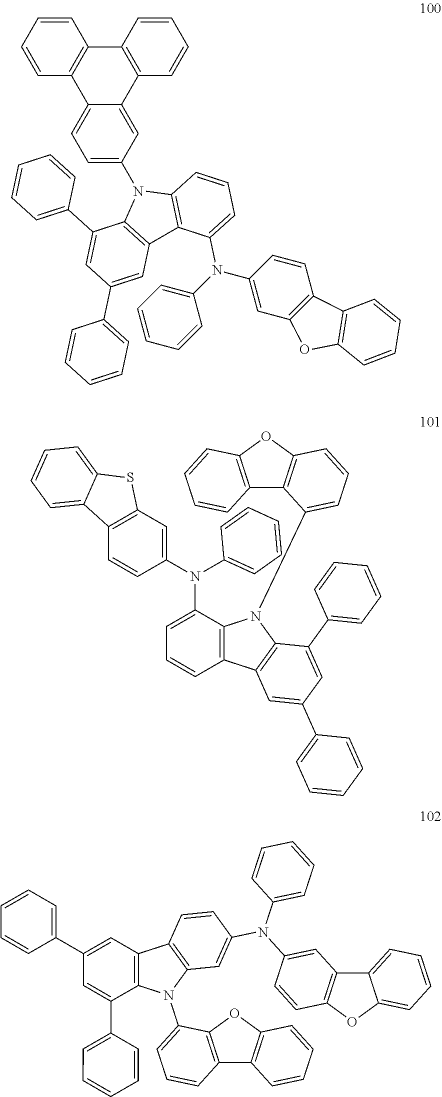 [SMILES: c1ccc(-c2cc(-c3ccccc3)c3c(c2)c2c(N(c4ccccc4)c4ccc5c(c4)oc4ccccc45)cccc2n3-c2ccc3c4ccccc4c4ccccc4c3c2)cc1 ; c1ccc(-c2cc(-c3ccccc3)c3c(c2)c2cccc(N(c4ccccc4)c4ccc5c(c4)sc4ccccc45)c2n3-c2cccc3oc4ccccc4c23)cc1 ; c1ccc(-c2cc(-c3ccccc3)c3c(c2)c2ccc(N(c4ccccc4)c4ccc5oc6ccccc6c5c4)cc2n3-c2cccc3c2oc2ccccc23)cc1]

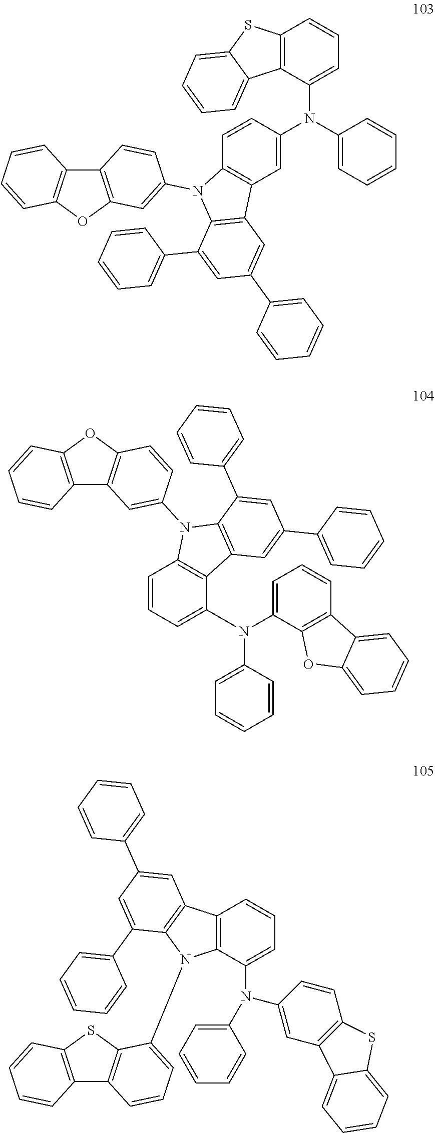 [SMILES: c1ccc(-c2cc(-c3ccccc3)c3c(c2)c2cc(N(c4ccccc4)c4cccc5sc6ccccc6c45)ccc2n3-c2ccc3c(c2)oc2ccccc23)cc1 ; c1ccc(-c2cc(-c3ccccc3)c3c(c2)c2c(N(c4ccccc4)c4cccc5c4oc4ccccc45)cccc2n3-c2ccc3oc4ccccc4c3c2)cc1 ; c1ccc(-c2cc(-c3ccccc3)c3c(c2)c2cccc(N(c4ccccc4)c4ccc5sc6ccccc6c5c4)c2n3-c2cccc3c2sc2ccccc23)cc1]

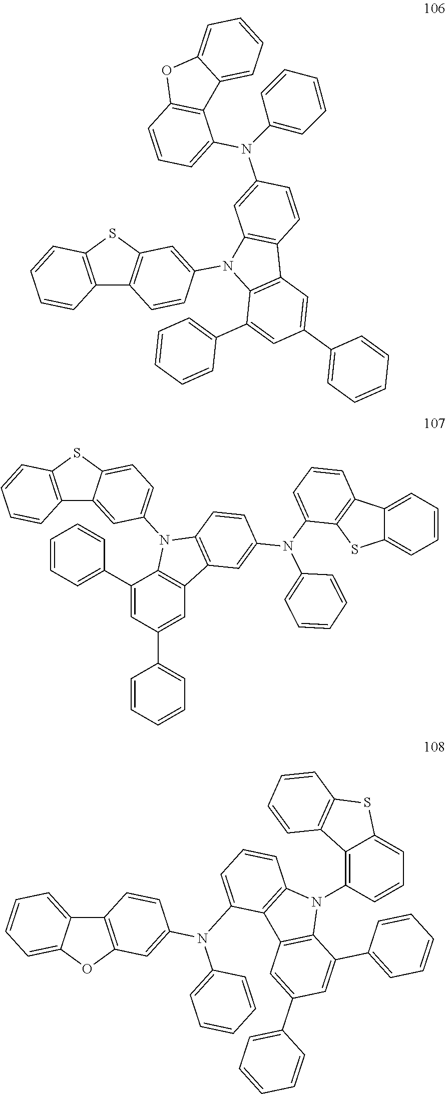 [SMILES: c1ccc(-c2cc(-c3ccccc3)c3c(c2)c2ccc(N(c4ccccc4)c4cccc5oc6ccccc6c45)cc2n3-c2ccc3c(c2)sc2ccccc23)cc1 ; c1ccc(-c2cc(-c3ccccc3)c3c(c2)c2cc(N(c4ccccc4)c4cccc5c4sc4ccccc45)ccc2n3-c2ccc3sc4ccccc4c3c2)cc1 ; c1ccc(-c2cc(-c3ccccc3)c3c(c2)c2c(N(c4ccccc4)c4ccc5c(c4)oc4ccccc45)cccc2n3-c2cccc3sc4ccccc4c23)cc1]

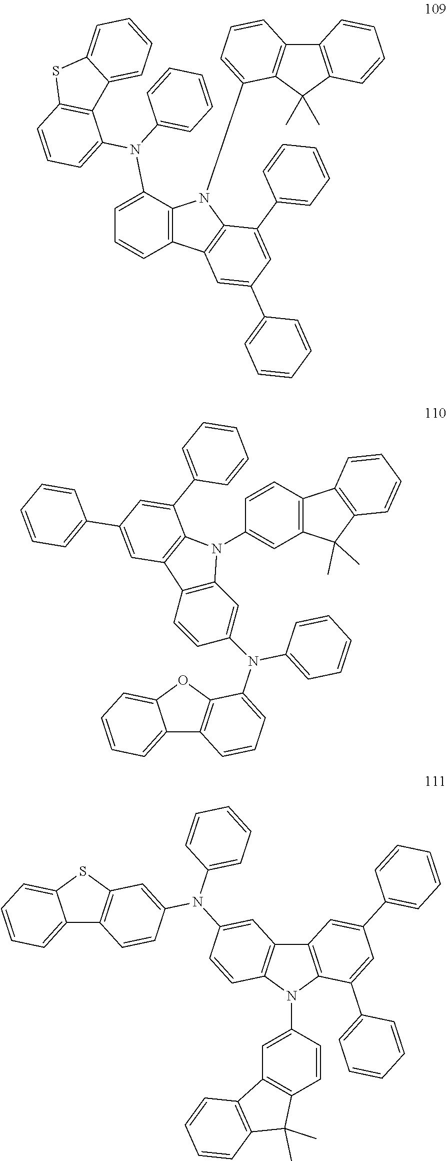 [SMILES: CC1(C)c2ccccc2-c2cccc(-n3c4c(-c5ccccc5)cc(-c5ccccc5)cc4c4cccc(N(c5ccccc5)c5cccc6sc7ccccc7c56)c43)c21 ; CC1(C)c2ccccc2-c2ccc(-n3c4cc(N(c5ccccc5)c5cccc6c5oc5ccccc56)ccc4c4cc(-c5ccccc5)cc(-c5ccccc5)c43)cc21 ; CC1(C)c2ccccc2-c2cc(-n3c4ccc(N(c5ccccc5)c5ccc6c(c5)sc5ccccc56)cc4c4cc(-c5ccccc5)cc(-c5ccccc5)c43)ccc21]

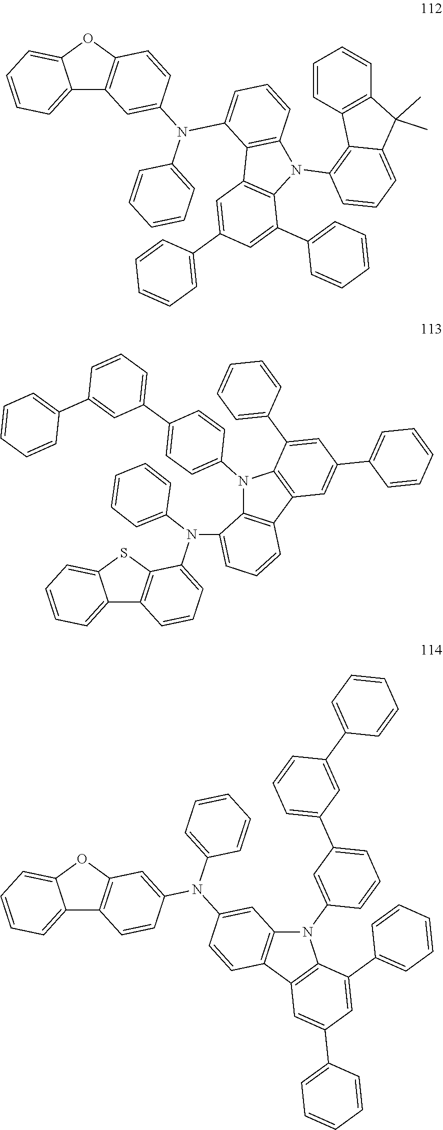 [SMILES: CC1(C)c2ccccc2-c2c(-n3c4cccc(N(c5ccccc5)c5ccc6oc7ccccc7c6c5)c4c4cc(-c5ccccc5)cc(-c5ccccc5)c43)cccc21 ; c1ccc(-c2cccc(-c3ccc(-n4c5c(-c6ccccc6)cc(-c6ccccc6)cc5c5cccc(N(c6ccccc6)c6cccc7c6sc6ccccc67)c54)cc3)c2)cc1 ; c1ccc(-c2cccc(-c3cccc(-n4c5cc(N(c6ccccc6)c6ccc7c(c6)oc6ccccc67)ccc5c5cc(-c6ccccc6)cc(-c6ccccc6)c54)c3)c2)cc1]

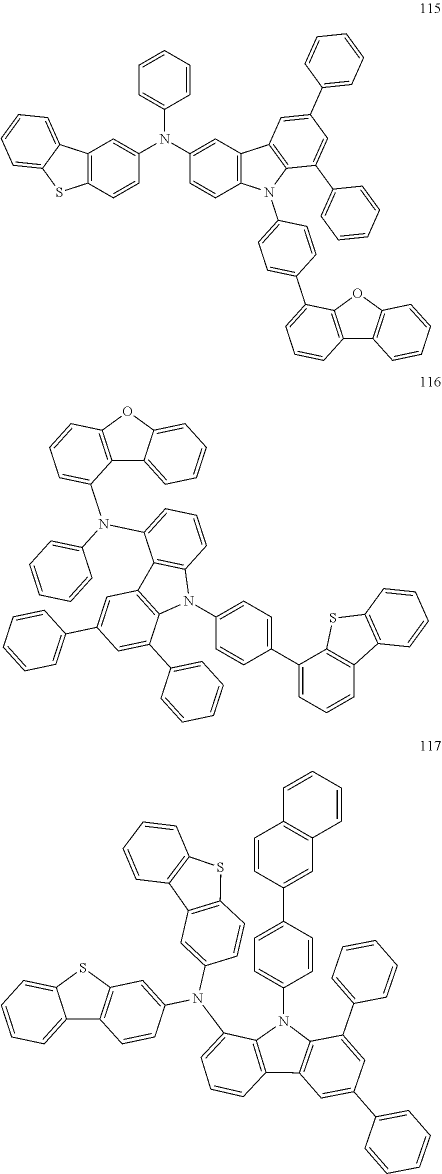 [SMILES: c1ccc(-c2cc(-c3ccccc3)c3c(c2)c2cc(N(c4ccccc4)c4ccc5sc6ccccc6c5c4)ccc2n3-c2ccc(-c3cccc4c3oc3ccccc34)cc2)cc1 ; c1ccc(-c2cc(-c3ccccc3)c3c(c2)c2c(N(c4ccccc4)c4cccc5oc6ccccc6c45)cccc2n3-c2ccc(-c3cccc4c3sc3ccccc34)cc2)cc1 ; c1ccc(-c2cc(-c3ccccc3)c3c(c2)c2cccc(N(c4ccc5c(c4)sc4ccccc45)c4ccc5sc6ccccc6c5c4)c2n3-c2ccc(-c3ccc4ccccc4c3)cc2)cc1]

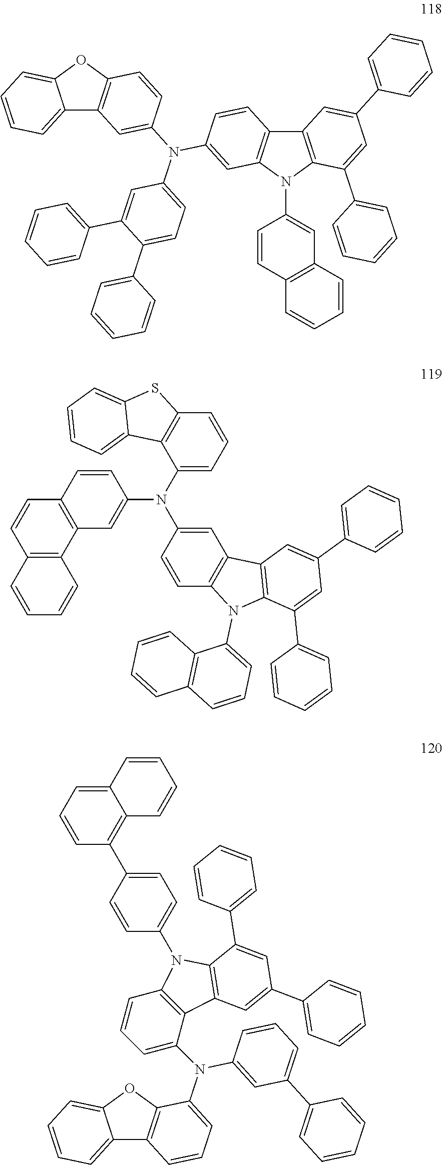 [SMILES: c1ccc(-c2cc(-c3ccccc3)c3c(c2)c2ccc(N(c4ccc(-c5ccccc5)c(-c5ccccc5)c4)c4ccc5oc6ccccc6c5c4)cc2n3-c2ccc3ccccc3c2)cc1 ; c1ccc(-c2cc(-c3ccccc3)c3c(c2)c2cc(N(c4ccc5ccc6ccccc6c5c4)c4cccc5sc6ccccc6c45)ccc2n3-c2cccc3ccccc23)cc1 ; c1ccc(-c2cccc(N(c3cccc4c3oc3ccccc34)c3cccc4c3c3cc(-c5ccccc5)cc(-c5ccccc5)c3n4-c3ccc(-c4cccc5ccccc45)cc3)c2)cc1]

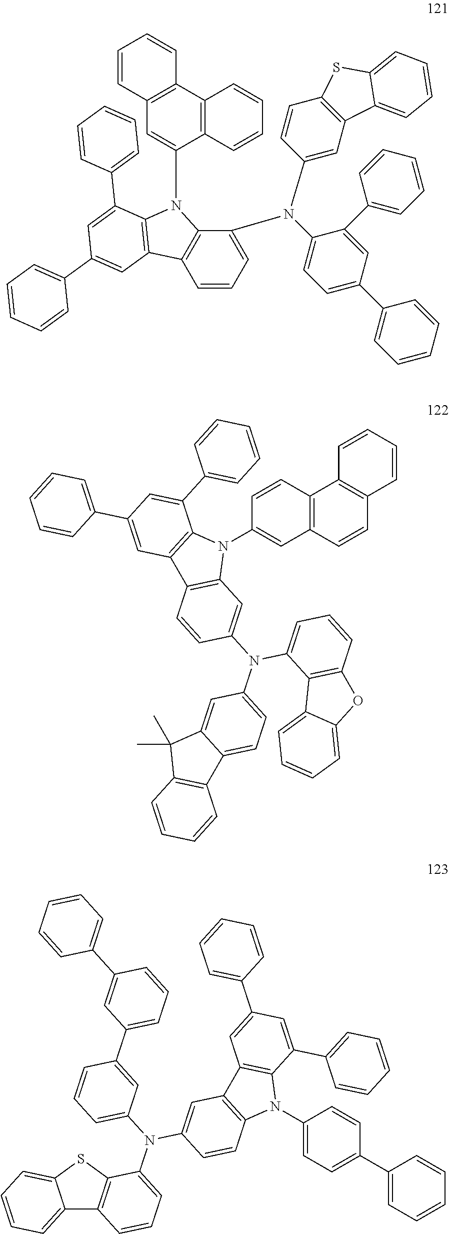 [SMILES: c1ccc(-c2ccc(N(c3ccc4sc5ccccc5c4c3)c3cccc4c5cc(-c6ccccc6)cc(-c6ccccc6)c5n(-c5cc6ccccc6c6ccccc56)c34)c(-c3ccccc3)c2)cc1 ; CC1(C)c2ccccc2-c2ccc(N(c3ccc4c5cc(-c6ccccc6)cc(-c6ccccc6)c5n(-c5ccc6c(ccc7ccccc76)c5)c4c3)c3cccc4oc5ccccc5c34)cc21 ; c1ccc(-c2ccc(-n3c4ccc(N(c5cccc(-c6cccc(-c7ccccc7)c6)c5)c5cccc6c5sc5ccccc56)cc4c4cc(-c5ccccc5)cc(-c5ccccc5)c43)cc2)cc1]

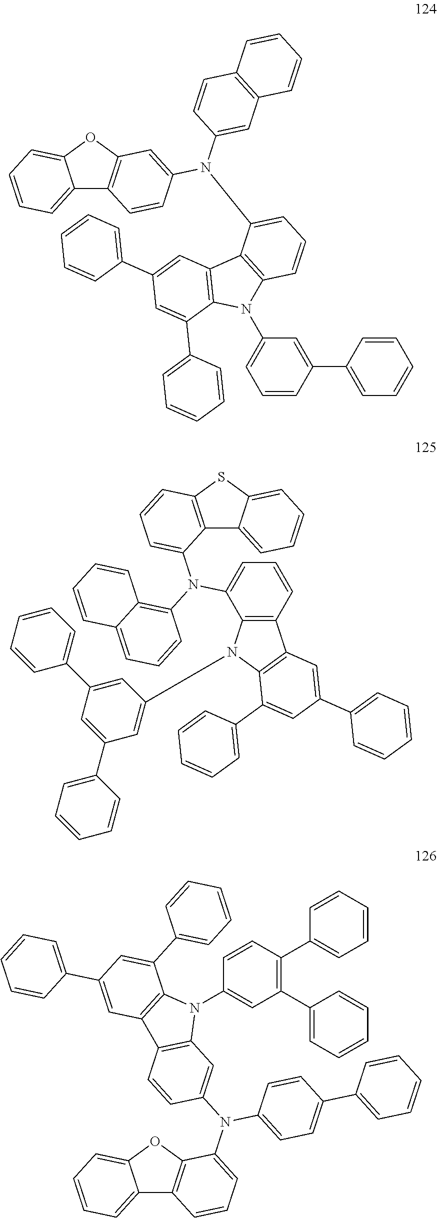 [SMILES: c1ccc(-c2cccc(-n3c4cccc(N(c5ccc6ccccc6c5)c5ccc6c(c5)oc5ccccc56)c4c4cc(-c5ccccc5)cc(-c5ccccc5)c43)c2)cc1 ; c1ccc(-c2cc(-c3ccccc3)cc(-n3c4c(-c5ccccc5)cc(-c5ccccc5)cc4c4cccc(N(c5cccc6ccccc56)c5cccc6sc7ccccc7c56)c43)c2)cc1 ; c1ccc(-c2ccc(N(c3ccc4c5cc(-c6ccccc6)cc(-c6ccccc6)c5n(-c5ccc(-c6ccccc6)c(-c6ccccc6)c5)c4c3)c3cccc4c3oc3ccccc34)cc2)cc1]

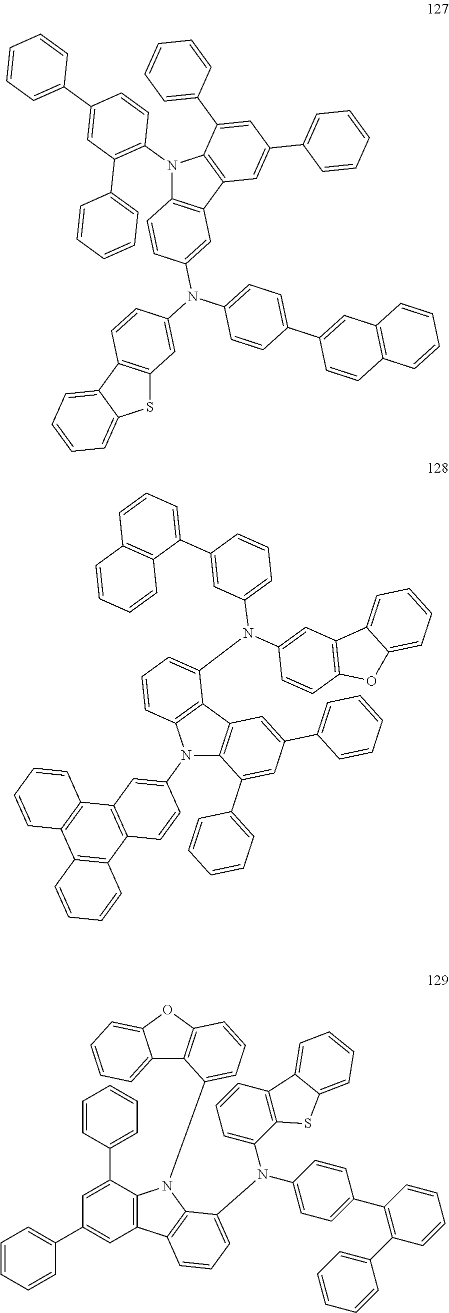 [SMILES: c1ccc(-c2ccc(-n3c4ccc(N(c5ccc(-c6ccc7ccccc7c6)cc5)c5ccc6c(c5)sc5ccccc56)cc4c4cc(-c5ccccc5)cc(-c5ccccc5)c43)c(-c3ccccc3)c2)cc1 ; c1ccc(-c2cc(-c3ccccc3)c3c(c2)c2c(N(c4cccc(-c5cccc6ccccc56)c4)c4ccc5oc6ccccc6c5c4)cccc2n3-c2ccc3c4ccccc4c4ccccc4c3c2)cc1 ; c1ccc(-c2cc(-c3ccccc3)c3c(c2)c2cccc(N(c4ccc(-c5ccccc5-c5ccccc5)cc4)c4cccc5c4sc4ccccc45)c2n3-c2cccc3oc4ccccc4c23)cc1]

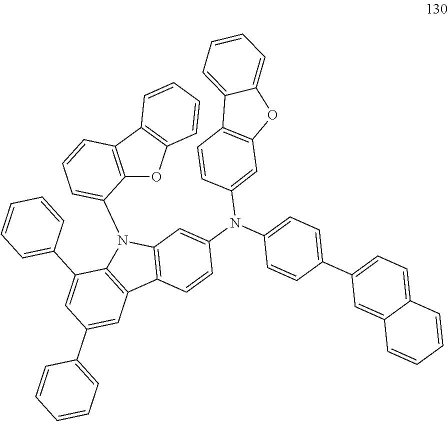 [SMILES: c1ccc(-c2cc(-c3ccccc3)c3c(c2)c2ccc(N(c4ccc(-c5ccc6ccccc6c5)cc4)c4ccc5c(c4)oc4ccccc45)cc2n3-c2cccc3c2oc2ccccc23)cc1]

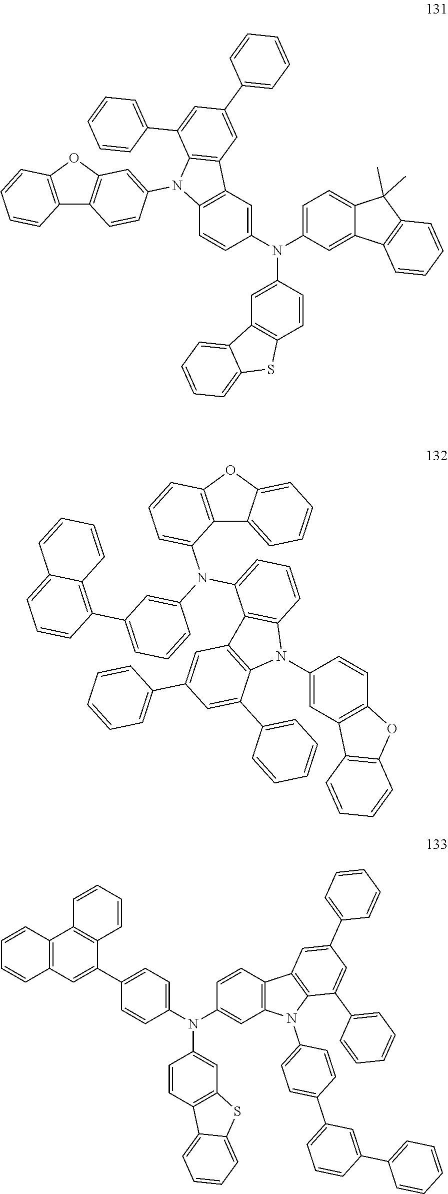 [SMILES: CC1(C)c2ccccc2-c2cc(N(c3ccc4sc5ccccc5c4c3)c3ccc4c(c3)c3cc(-c5ccccc5)cc(-c5ccccc5)c3n4-c3ccc4c(c3)oc3ccccc34)ccc21 ; c1ccc(-c2cc(-c3ccccc3)c3c(c2)c2c(N(c4cccc(-c5cccc6ccccc56)c4)c4cccc5oc6ccccc6c45)cccc2n3-c2ccc3oc4ccccc4c3c2)cc1 ; c1ccc(-c2cccc(-c3ccc(-n4c5cc(N(c6ccc(-c7cc8ccccc8c8ccccc78)cc6)c6ccc7c(c6)sc6ccccc67)ccc5c5cc(-c6ccccc6)cc(-c6ccccc6)c54)cc3)c2)cc1]

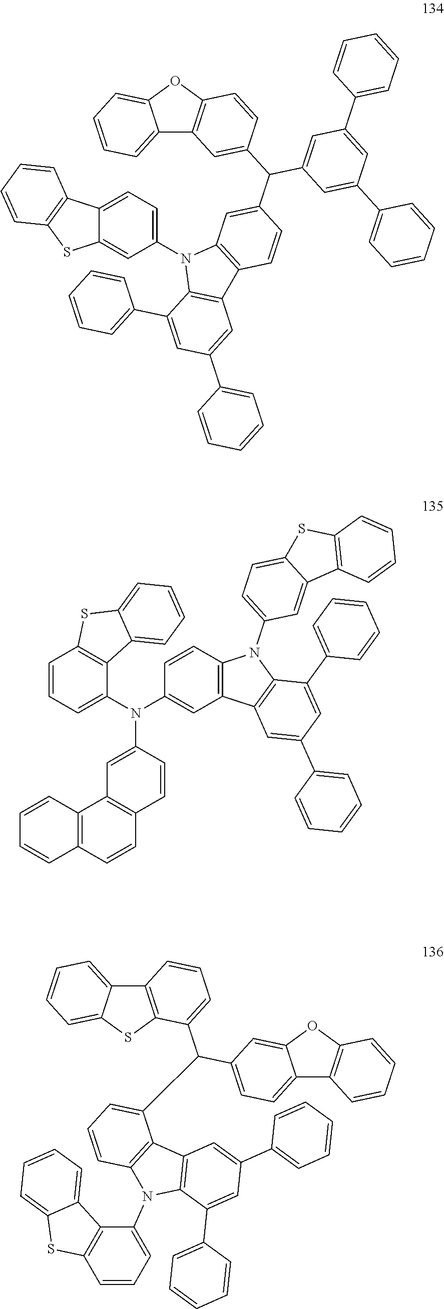 [SMILES: c1ccc(-c2cc(-c3ccccc3)cc(C(c3ccc4oc5ccccc5c4c3)c3ccc4c5cc(-c6ccccc6)cc(-c6ccccc6)c5n(-c5ccc6c(c5)sc5ccccc56)c4c3)c2)cc1 ; c1ccc(-c2cc(-c3ccccc3)c3c(c2)c2cc(N(c4ccc5ccc6ccccc6c5c4)c4cccc5sc6ccccc6c45)ccc2n3-c2ccc3sc4ccccc4c3c2)cc1 ; c1ccc(-c2cc(-c3ccccc3)c3c(c2)c2c(C(c4ccc5c(c4)oc4ccccc45)c4cccc5c4sc4ccccc45)cccc2n3-c2cccc3sc4ccccc4c23)cc1]

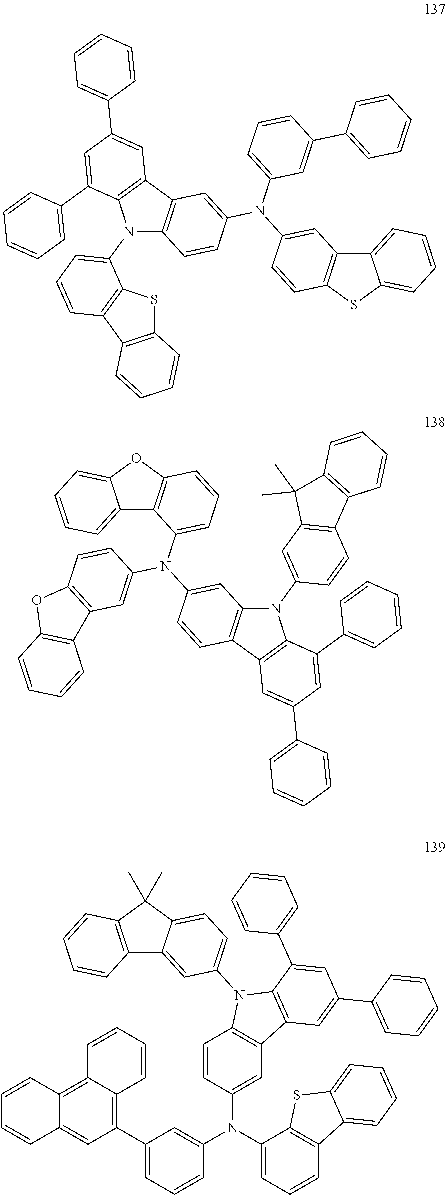 [SMILES: c1ccc(-c2cccc(N(c3ccc4sc5ccccc5c4c3)c3ccc4c(c3)c3cc(-c5ccccc5)cc(-c5ccccc5)c3n4-c3cccc4c3sc3ccccc34)c2)cc1 ; CC1(C)c2ccccc2-c2ccc(-n3c4cc(N(c5ccc6oc7ccccc7c6c5)c5cccc6oc7ccccc7c56)ccc4c4cc(-c5ccccc5)cc(-c5ccccc5)c43)cc21 ; CC1(C)c2ccccc2-c2cc(-n3c4ccc(N(c5cccc(-c6cc7ccccc7c7ccccc67)c5)c5cccc6c5sc5ccccc56)cc4c4cc(-c5ccccc5)cc(-c5ccccc5)c43)ccc21]

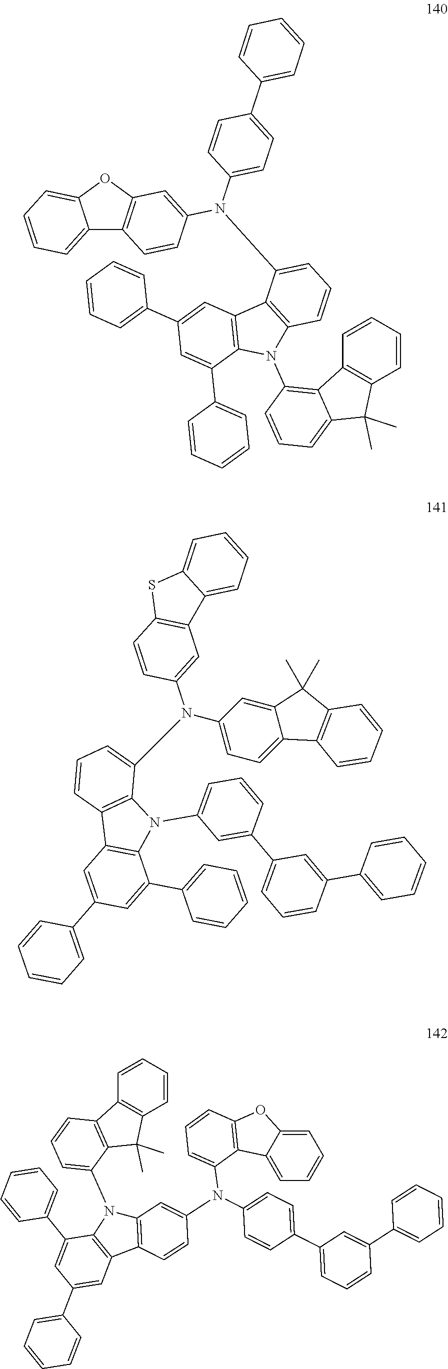 [SMILES: CC1(C)c2ccccc2-c2c(-n3c4cccc(N(c5ccc(-c6ccccc6)cc5)c5ccc6c(c5)oc5ccccc56)c4c4cc(-c5ccccc5)cc(-c5ccccc5)c43)cccc21 ; CC1(C)c2ccccc2-c2ccc(N(c3ccc4sc5ccccc5c4c3)c3cccc4c5cc(-c6ccccc6)cc(-c6ccccc6)c5n(-c5cccc(-c6cccc(-c7ccccc7)c6)c5)c34)cc21 ; CC1(C)c2ccccc2-c2cccc(-n3c4cc(N(c5ccc(-c6cccc(-c7ccccc7)c6)cc5)c5cccc6oc7ccccc7c56)ccc4c4cc(-c5ccccc5)cc(-c5ccccc5)c43)c21]

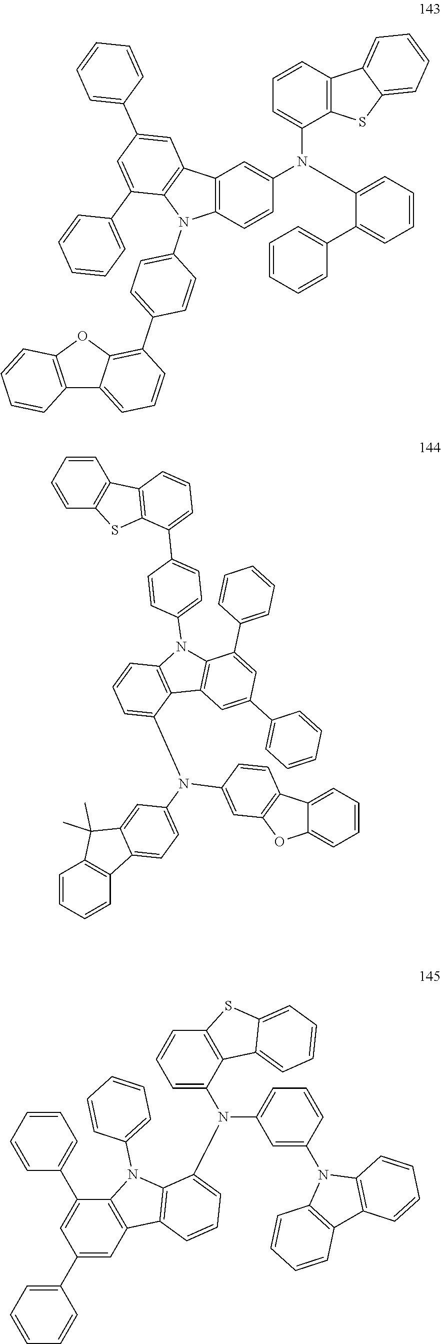 [SMILES: c1ccc(-c2cc(-c3ccccc3)c3c(c2)c2cc(N(c4ccccc4-c4ccccc4)c4cccc5c4sc4ccccc45)ccc2n3-c2ccc(-c3cccc4c3oc3ccccc34)cc2)cc1 ; CC1(C)c2ccccc2-c2ccc(N(c3ccc4c(c3)oc3ccccc34)c3cccc4c3c3cc(-c5ccccc5)cc(-c5ccccc5)c3n4-c3ccc(-c4cccc5c4sc4ccccc45)cc3)cc21 ; c1ccc(-c2cc(-c3ccccc3)c3c(c2)c2cccc(N(c4cccc(-n5c6ccccc6c6ccccc65)c4)c4cccc5sc6ccccc6c45)c2n3-c2ccccc2)cc1]

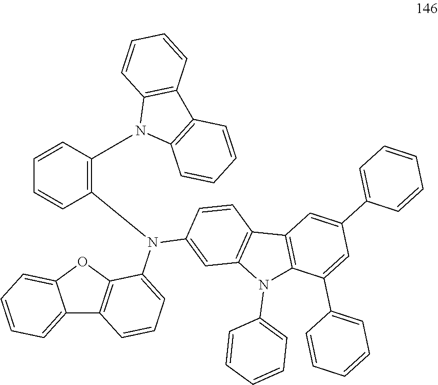 [SMILES: c1ccc(-c2cc(-c3ccccc3)c3c(c2)c2ccc(N(c4ccccc4-n4c5ccccc5c5ccccc54)c4cccc5c4oc4ccccc45)cc2n3-c2ccccc2)cc1]

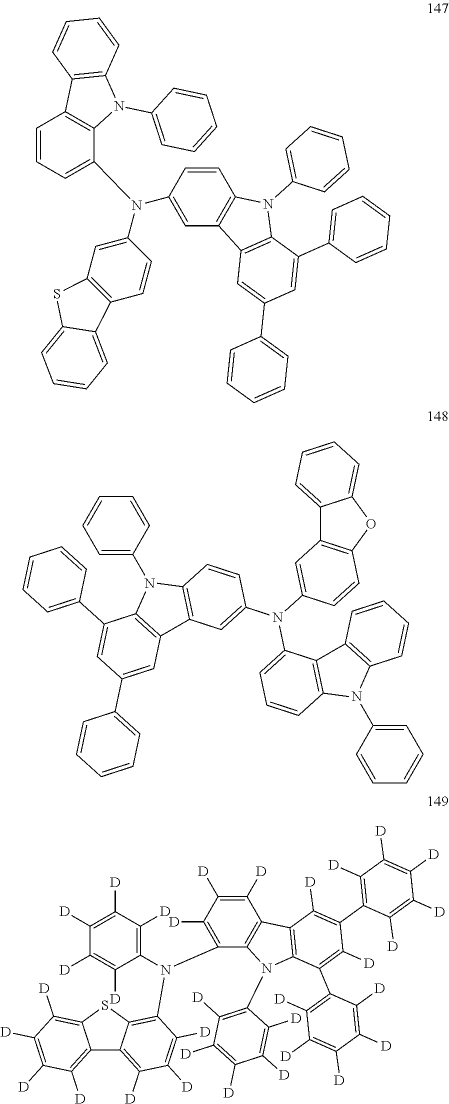 [SMILES: c1ccc(-c2cc(-c3ccccc3)c3c(c2)c2cc(N(c4ccc5c(c4)sc4ccccc45)c4cccc5c6ccccc6n(-c6ccccc6)c45)ccc2n3-c2ccccc2)cc1 ; c1ccc(-c2cc(-c3ccccc3)c3c(c2)c2cc(N(c4ccc5oc6ccccc6c5c4)c4cccc5c4c4ccccc4n5-c4ccccc4)ccc2n3-c2ccccc2)cc1 ; [2H]c1c([2H])c([2H])c(-c2c([2H])c(-c3c([2H])c([2H])c([2H])c([2H])c3[2H])c3c(c2[2H])c2c([2H])c([2H])c([2H])c(N(c4c([2H])c([2H])c([2H])c([2H])c4[2H])c4c([2H])c([2H])c([2H])c5c4sc4c([2H])c([2H])c([2H])c([2H])c45)c2n3-c2c([2H])c([2H])c([2H])c([2H])c2[2H])c([2H])c1[2H]]

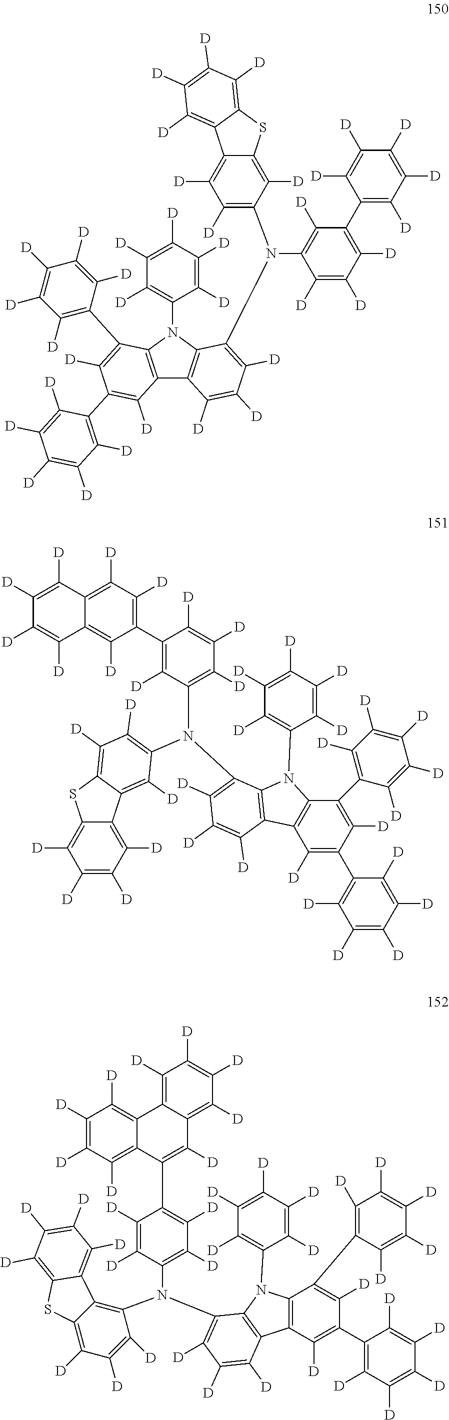

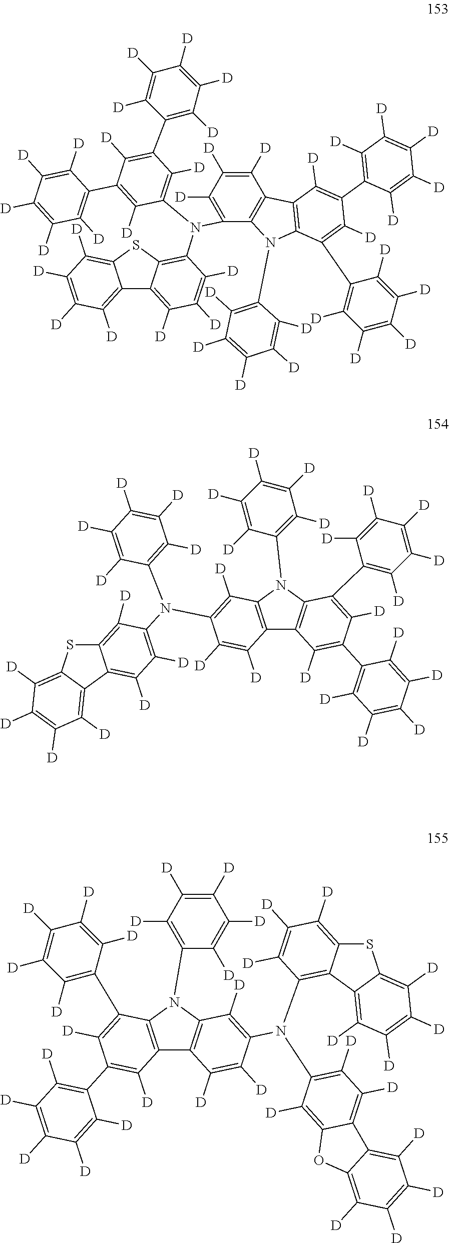

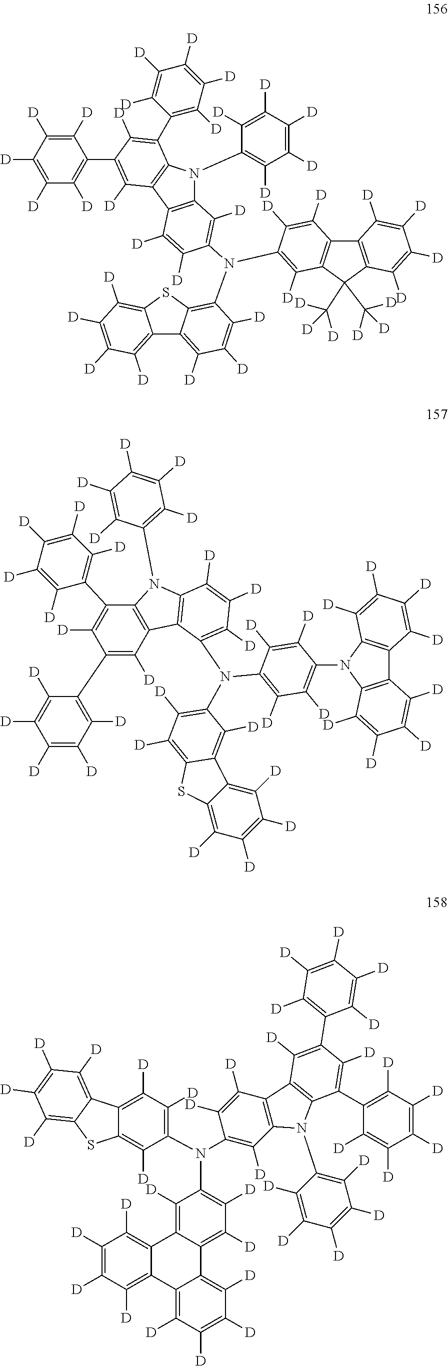

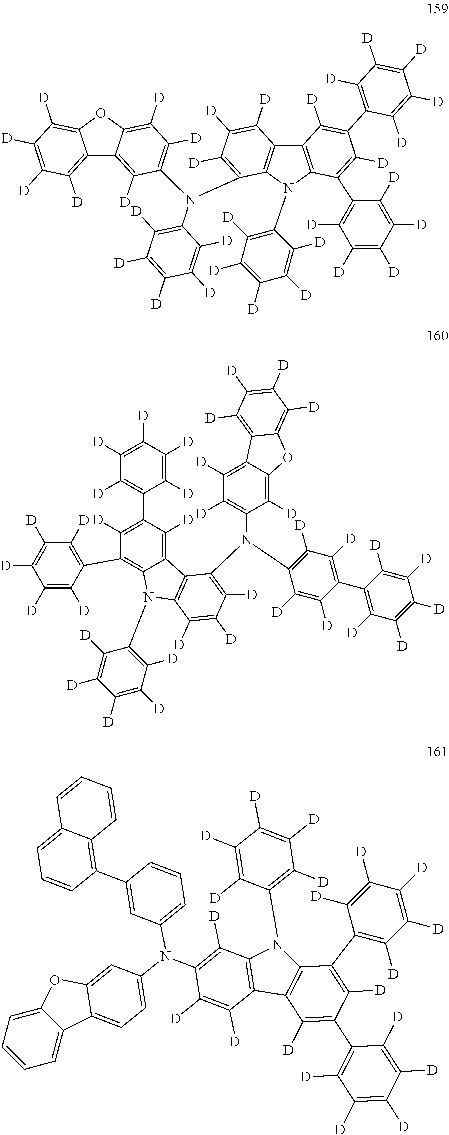

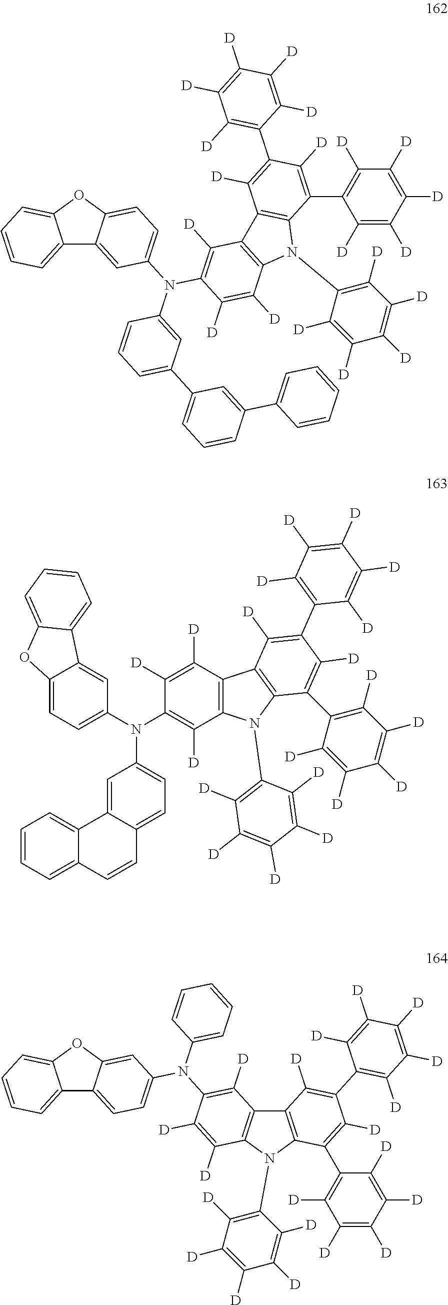

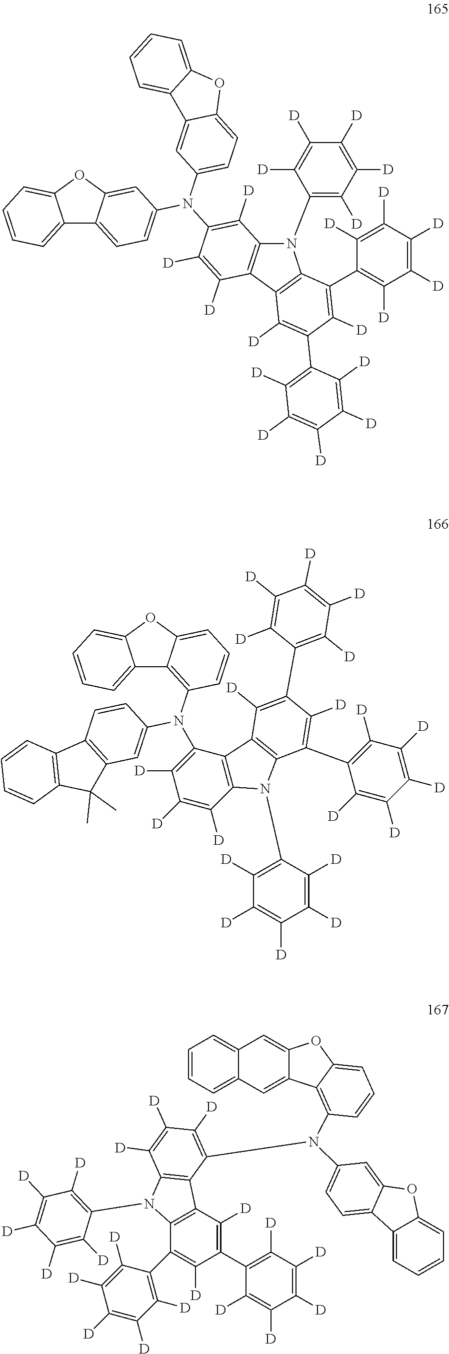

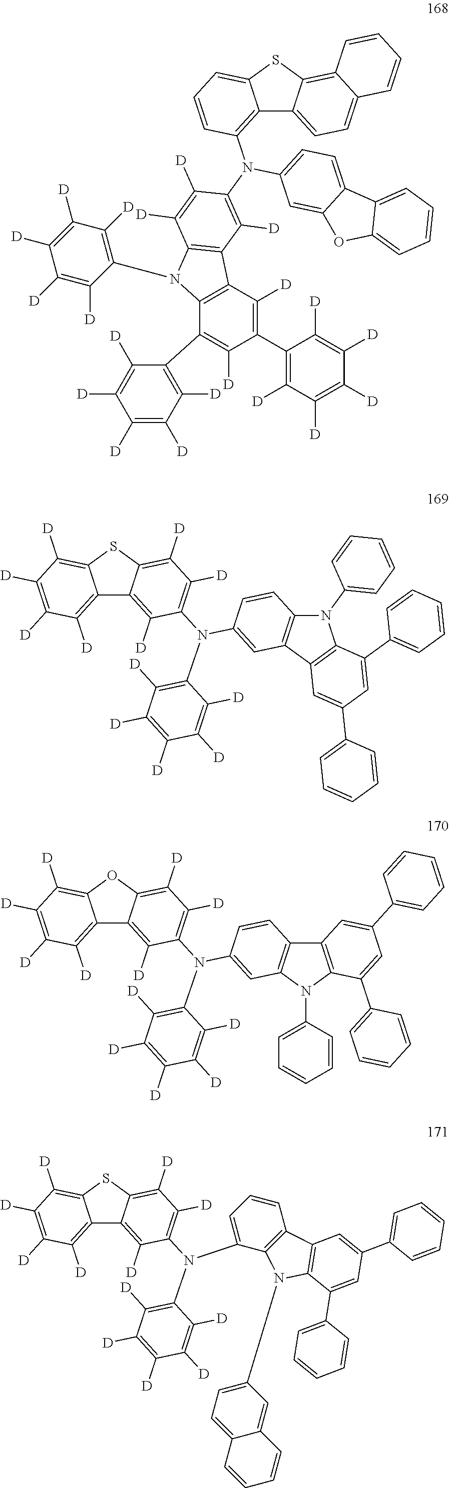

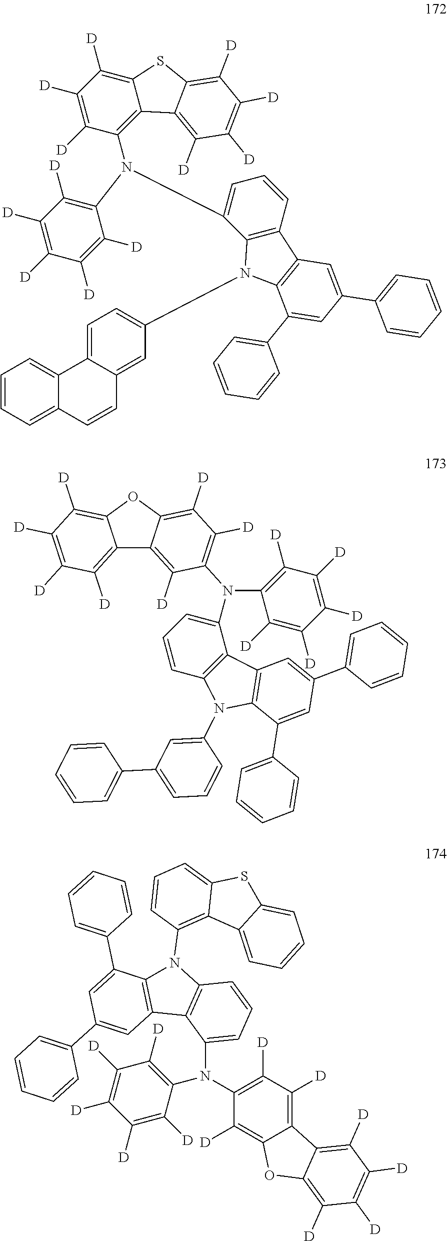 [SMILES: [2H]c1c([2H])c([2H])c(N(c2c([2H])c([2H])c([2H])c3sc4c([2H])c([2H])c([2H])c([2H])c4c23)c2cccc3c4cc(-c5ccccc5)cc(-c5ccccc5)c4n(-c4ccc5c(ccc6ccccc65)c4)c23)c([2H])c1[2H] ; [2H]c1c([2H])c([2H])c(N(c2c([2H])c([2H])c3oc4c([2H])c([2H])c([2H])c([2H])c4c3c2[2H])c2cccc3c2c2cc(-c4ccccc4)cc(-c4ccccc4)c2n3-c2cccc(-c3ccccc3)c2)c([2H])c1[2H] ; [2H]c1c([2H])c([2H])c(N(c2c([2H])c([2H])c3c(oc4c([2H])c([2H])c([2H])c([2H])c43)c2[2H])c2cccc3c2c2cc(-c4ccccc4)cc(-c4ccccc4)c2n3-c2cccc3sc4ccccc4c23)c([2H])c1[2H]]

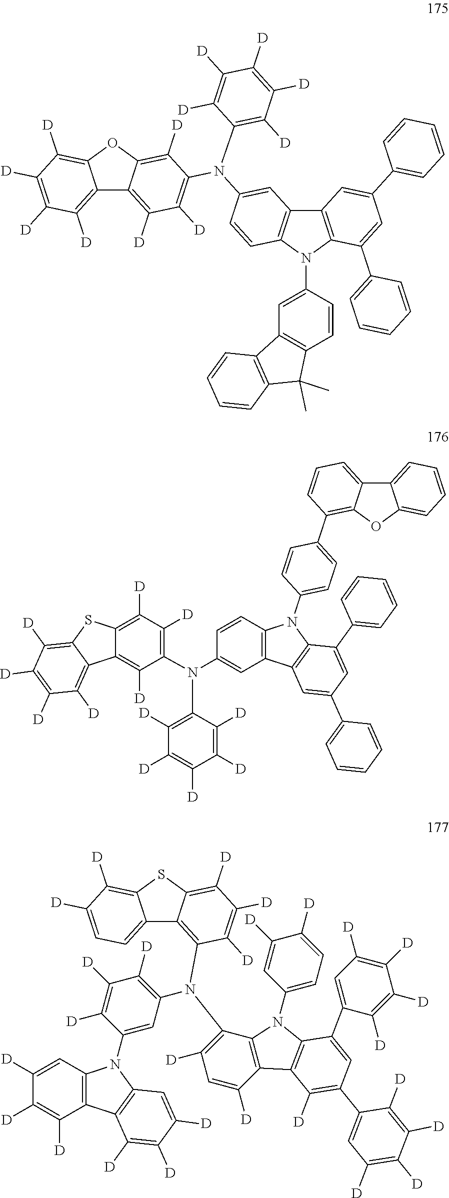

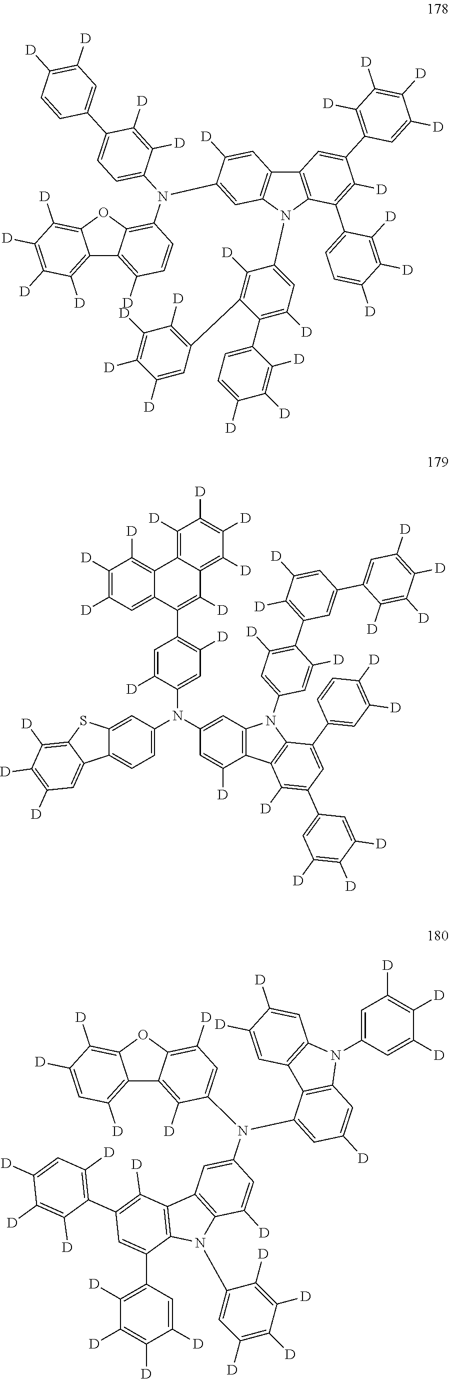

As long as the unique characteristics of the heterocyclic compound represented by Chemical Formula 1 are maintained, various substituents may be introduced in addition to the structures exemplified above to synthesize a heterocyclic compound to which the characteristics of the introduced substituents are added. For example, it is possible to synthesize a material which satisfies the conditions required for each organic material layer by introducing into the core structure a substituent usually used for a hole injection layer material, a hole transport layer material, a hole transport auxiliary layer material, an emission layer material, an electron transport layer material, an electron transport auxiliary layer material, and an electron blocking layer material used during the manufacture of an organic light emitting device.

In addition, it is possible to finely adjust an energy band-gap by introducing various substituents into the heterocyclic compound structure represented by Chemical Formula 1, and meanwhile, it is possible to improve characteristics at the interface between organic materials and diversify the use of the material.

### <Organic Light Emitting Device>

The organic light emitting device according to an exemplary embodiment of the present specification is an organic light emitting device including: a first electrode; a second electrode provided to face the first electrode; and one or more organic material layers provided between the first electrode and the second electrode, and one or more layers of the organic material layers may include the above-described heterocyclic compound (represented by Chemical Formula 1).

According to an exemplary embodiment of the present specification, the organic material layer further includes at least one of a hole transport layer and an electron blocking layer, and at least one of the hole transport layer and the electron blocking layer may include the heterocyclic compound (represented by Chemical Formula 1).

According to an exemplary embodiment of the present specification, the organic material layer further includes a hole transport layer, and the hole transport layer may include the heterocyclic compound (represented by Chemical Formula 1).

According to an exemplary embodiment of the present specification, the organic material layer further includes an electron blocking layer, and the electron blocking layer may include the heterocyclic compound (represented by Chemical Formula 1).

According to an exemplary embodiment of the present specification, the organic material layer further includes an emission layer, and the emission layer may include the heterocyclic compound represented by Chemical Formula 1.

In another exemplary embodiment of the present specification, the emission layer may include the heterocyclic compound represented by Chemical Formula 1 as a host.

According to an exemplary embodiment of the present specification, the emission layer may include the heterocyclic compound represented by Chemical Formula 1 as a red host.

According to an exemplary embodiment of the present specification, the emission layer may include the heterocyclic compound represented by Chemical Formula 1 as a green host.

According to an exemplary embodiment of the present specification, the emission layer may include the heterocyclic compound represented by Chemical Formula 1 as a blue host.

According to an exemplary embodiment of the present specification, the first electrode may be a positive electrode, and the second electrode may be a negative electrode.

According to an exemplary embodiment of the present specification, the first electrode may be a negative electrode, and the second electrode may be a positive electrode.

According to an exemplary embodiment of the present specification, the organic light emitting device may be a blue organic light emitting device, and the heterocyclic compound represented by Chemical Formula 1 may be used as a material for the blue organic light emitting device.

According to an exemplary embodiment of the present specification, the organic light emitting device may be a green organic light emitting device, and the heterocyclic compound represented by Chemical Formula 1 may be used as a material for the green organic light emitting device.

According to an exemplary embodiment of the present specification, the organic light emitting device may be a red organic light emitting device, and the heterocyclic compound represented by Chemical Formula 1 may be used as a material for the red organic light emitting device.

The organic material layer of the organic light emitting device of the present specification may also have a single-layered structure, but may have a multi-layered structure in which two or more organic material layers are stacked. For example, the organic light emitting device of the present specification may have a structure including a hole injection layer, a hole transport layer, an emission layer, an electron transport layer, an electron injection layer, and the like as organic material layers. However, the structure of the organic light emitting device is not limited thereto, and may include less or more numbers of organic material layers.

According to an exemplary embodiment of the present specification, the organic material layer may include an iridium-based dopant.

According to an exemplary embodiment of the present specification, as the iridium-based dopant, Ir(ppy)₃, which is a green phosphorescent dopant, may be used, but the iridium-based dopant is not limited thereto.

According to an exemplary embodiment of the present specification, as the iridium-based dopant, (piq)₂(Ir) (acac), which is a red phosphorescent dopant, may be used, but the iridium-based dopant is not limited thereto.

In the organic light emitting device of the present specification, as a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

In the organic light emitting device of the present specification, as a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

In the organic light emitting device of the present specification, as a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

In the organic light emitting device of the present specification, as a hole transport material, a pyrazoline derivative, an arylamine-based derivative, a stilbene derivative, a triphenyldiamine derivative, and the like may be used, and a low-molecular weight or polymer material may also be used.

In the organic light emitting device of the present specification, as an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

In the organic light emitting device of the present specification, as an electron injection material, for example, LiF is representatively used in the art, but the present specification is not limited thereto.

In the organic light emitting device of the present specification, as a light emitting material, a red, green, or blue light emitting material may be further used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, two or more types of materials selected from N-type host materials or P-type host materials may be used as a host material for an emission layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

The organic light emitting device of the present specification may further include one or two or more layers selected from the group consisting of an emission layer, a hole injection layer, a hole transport layer, a hole transport auxiliary layer, an electron injection layer, an electron transport layer, an electron blocking layer, and a hole blocking layer.

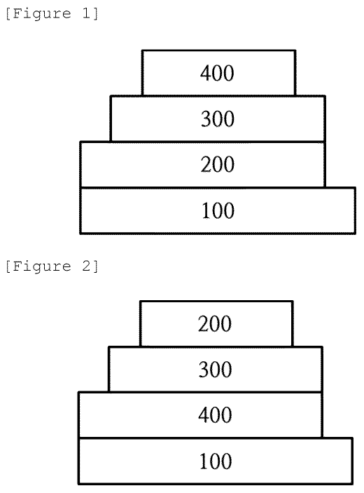

FIGS. 1 to 3 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

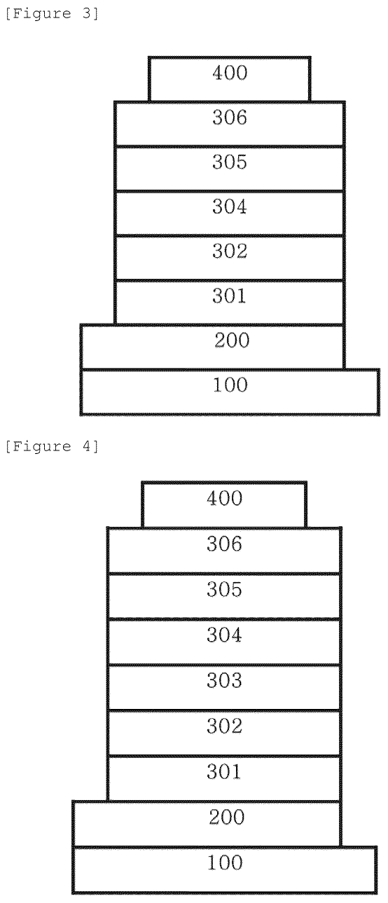

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as illustrated in FIG. 2, an organic light emitting device in which a negative electrode, an organic material layer, and a positive electrode are sequentially stacked on a substrate may also be implemented. The composition for an organic light emitting device may be included in the organic material layer 300, and the organic material layer 300 may be one or more layers.

FIG. 3 exemplifies a case where a positive electrode 200 is provided on a substrate 100 and an organic material layer is provided between the positive electrode 200 and a negative electrode 400 in a multi-layer structure. The organic light emitting device according to FIG. 3 may include a hole injection layer 301, a hole transport layer 302, an emission layer 304, an electron transport layer 305, and an electron injection layer 306 as an organic material layer, and the organic light emitting device according to FIG. 4 may further include an electron blocking layer 303 between the hole transport layer 302 and the emission layer 304. The heterocyclic compound (represented by Chemical Formula 1) may be included in the hole transport layer 302 and/or the electron blocking layer 303.

The scope of the present application is not limited by the stacking structure as described above, and if necessary, the other layers except for the hole transport layer and/or the electron blocking layer may be omitted, and another necessary functional layer may be further added.

The organic light emitting device according to an exemplary embodiment of the present specification includes a first electrode; a first stack provided on the first electrode and including a first emission layer; a second stack provided on the first stack and including a second emission layer; and a second electrode provided on the second stack.

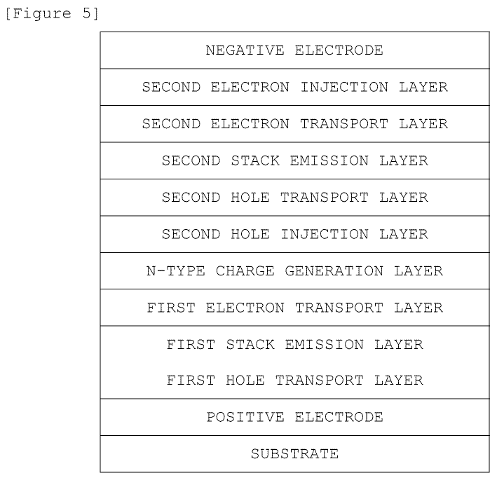

According to FIG. 5, when a first electrode on a substrate is a positive electrode, a first stack is provided on the positive electrode, a second stack is provided on the first stack, and a second electrode on the second stack may be a negative electrode. The first stack sequentially includes a first hole transport layer, a first stack emission layer (also referred to as a first emission layer), and a first electron transport layer, and the second stack may sequentially include a second hole injection layer, a second hole transport layer, a second stack emission layer (also referred to as a second emission layer), a second electron transport layer, and a second electron injection layer. An N-type charge generation layer may be interposed between the first stack and the second stack. Here, the heterocyclic compound represented by Chemical Formula 1 may be included in the second hole transport layer. Alternatively, the heterocyclic compound represented by Chemical Formula 1 may be included in the second electron injection layer.

Furthermore, the first stack and the second stack may each independently further include one or more of the above-described hole injection layer, hole transport layer, emission layer, hole blocking layer, electron transport layer, electron injection layer, and the like.

The heterocyclic compound represented by Chemical Formula 1 may be used in forming an organic material layer of an organic light emitting device, and may be more preferably used particularly as an electron blocking or hole transport material.

If necessary, when a different type of compound other than the heterocyclic compound represented by Chemical Formula 1 is mixed to form a mixture, the mixture may be in a premixed form, and a powder-state material may be mixed before forming the organic material layer of the organic light emitting device, and a compound that is in a liquid state at or above a suitable temperature may be mixed. The composition is in a solid state at a temperature which is equal to or less than the melting point of each material, and may be maintained as a liquid phase when the temperature is adjusted.

The heterocyclic compound represented by Chemical Formula 1 may additionally include materials publicly known in the art, such as solvents and additives.

### <Method for Manufacturing Organic Light Emitting Device>

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the heterocyclic compound (represented by Chemical Formula 1 above) according to an exemplary embodiment of the present specification.

According to an exemplary embodiment of the present specification, in the forming of the organic material layer, the heterocyclic compound represented by Chemical Formula 1 may be formed using a thermal vacuum deposition method.

The organic light emitting device according to an exemplary embodiment of the present specification may be manufactured by typical manufacturing methods and materials of the organic light emitting device, except that the above-described heterocyclic compound is used to form an organic material layer.

Specifically, for a method of forming an organic material layer, an organic material layer may be formed by not only a vacuum deposition method, but also a solution application method when the organic light emitting device is manufactured. Herein, the solution coating method means spin coating, dip coating, inkjet printing, screen printing, a spray method, roll coating, and the like, but is not limited thereto.

Hereinafter, the present specification will be described in more detail through Examples, but these Examples are provided only for exemplifying the present application, and are not intended to limit the scope of the present application.

### PREPARATION EXAMPLES

### [Preparation Example 1] Preparation of Compound 001

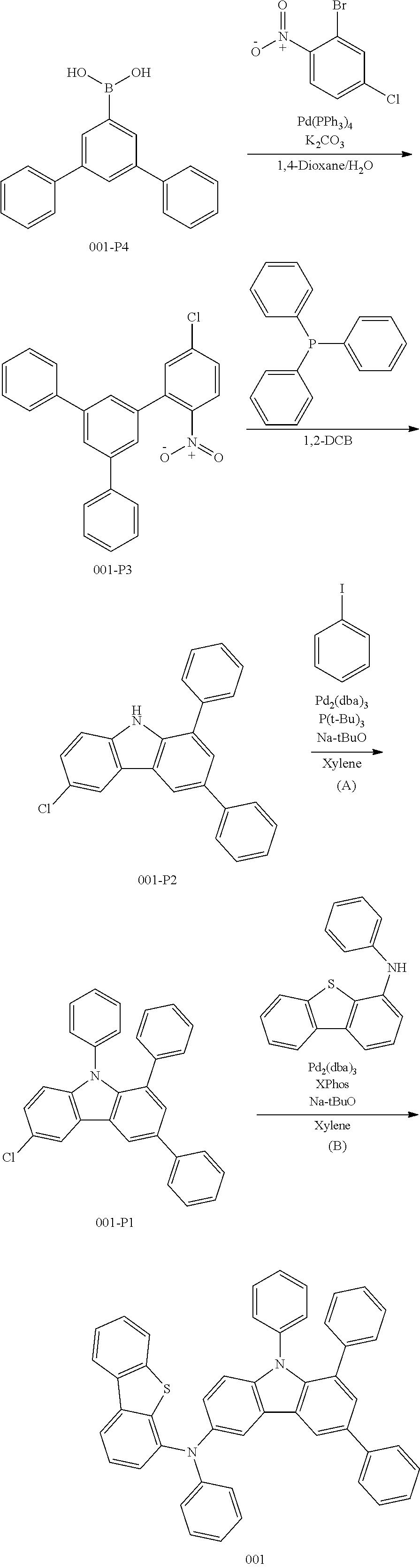

### 1) Preparation of Compound 001-P3

2-Bromo-4-chloro-1-nitrobenzene (50 g, 211.46 mmol, 1 eq), [1,1′:3′,1″-terphenyl]-5′-ylboronic acid (60.86 g, 222.03 mmol, 1.05 eq), Pd(PPh₃)₄ (12.22 g, 10.57 mmol, 0.05 eq), K₂CO₃ (73.06 g, 528.65 mmol, 2.5 eq), 1,4-dioxane (600 ml), and water (150 ml) were put into a container, and the resulting mixture was stirred at 120° C. for 6 hours.

After the reaction was terminated by adding water, extraction was performed using MC and water. Thereafter, moisture was removed with MgSO₄. The residue was separated by silica gel column to obtain 65 g of Compound 001-P3 with a yield of 79.67%.

### 2) Preparation of Compound 001-P2

Compound 001-P3 (65 g, 168.46 mmol, 1 eq), triphenylphosphine (110.47 g, 421.16 mmol, 2.5 eq), and 1,2-dichlorobenzene (650 ml) were put into a container, and the resulting mixture was stirred at 180° C. for 4 hours.

After the reaction was terminated by adding water, extraction was performed using MC and water. Thereafter, moisture was removed with MgSO₄. The residue was separated by silica gel column to obtain 39 g of Compound 001-P2 with a yield of 65.43%.

### 3) Preparation of Compound 001-P1

Compound 001-P2 (39 g, 110.22 mmol, 1 eq), iodobenzene (A) (68.14 g, 330.66 mmol, 3 eq), P(t-Bu)₃ (2.14 mL, 4.41 mmol, 0.04 eq), Pd₂(dba)₃ (2.51 g, 2.74 mmol, 0.02 eq), NaOtBu (21.18 g, 220.44 mmol, 2 eq), and xylene (400 ml) were put into a container, and the resulting mixture was stirred at 140° C. for 1 hour.

After the reaction was terminated by adding water, extraction was performed using MC and water. Thereafter, moisture was removed with MgSO₄. The residue was separated by silica gel column to obtain 34 g of Compound 001-P1 with a yield of 71.75%.

### 4) Preparation of Compound 001

Compound 001-P1 (10 g, 23.26 mmol, 1 eq), N-phenyldibenzo[b,d]thiophen-4-amine (B) (6.79 g, 24.42 mmol, 1.05 eq), XPhos (0.45 g, 0.93 mmol, 0.04 eq), Pd₂(dba)₃ (0.53 g, 0.58 mmol, 0.02 eq), NaOtBu (4.47 g, 46.52 mmol, 2 eq), and xylene (100 ml) were put into a container, and the resulting mixture was stirred at 140° C. for 1 hour.

After the reaction was terminated by adding water, extraction was performed using MC and water. Thereafter, moisture was removed with MgSO₄. The residue was separated by silica gel column to obtain 11.8 g of Compound 001 with a yield of 75.85%.

Each compound was synthesized in the same manner as in Preparation Example 1 above using Intermediate A in the following Table 1 and Intermediate B in the following Table 1 instead of iodobenzene (A) and 2-N-phenyldibenzo[b,d]thiophen-4-amine (B), respectively.

TABLE 1

| | Intermediate | Intermediate | |
|---|---|---|---|
| Compound No. | A | B | Yield |
| 003 | 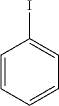 | 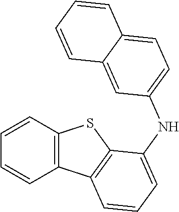 | 86% |
| 004 | 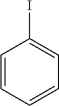 | 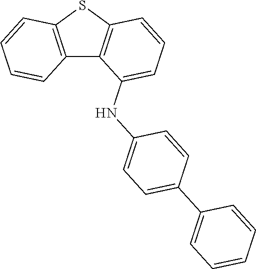 | 72% |
| 005 | 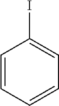 | 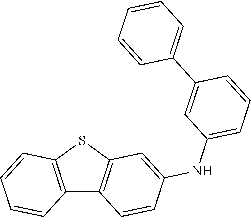 | 83% |
| 006 | 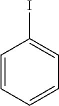 | 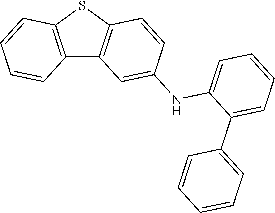 | 81% |
| 012 | 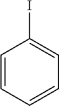 | 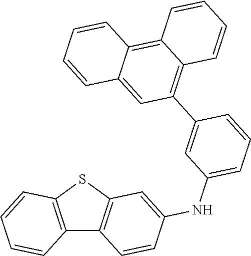 | 77% |
| 016 | 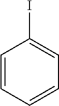 | 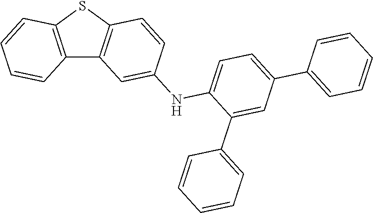 | 78% |
| 025 | 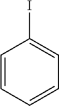 | 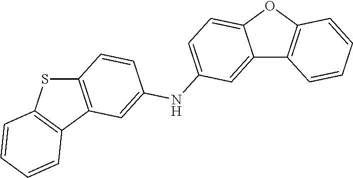 | 84% |
| 032 | 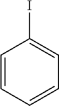 | 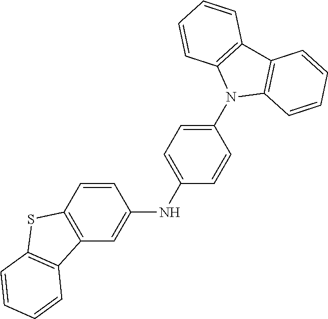 | 89% |
| 033 | 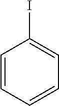 | 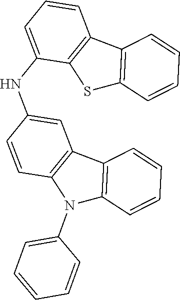 | 82% |
| 034 | 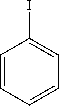 | 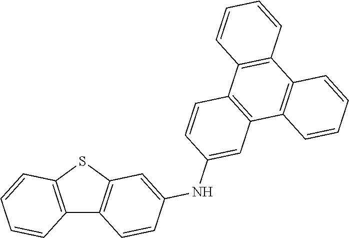 | 79% |
| 036 | 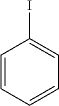 | 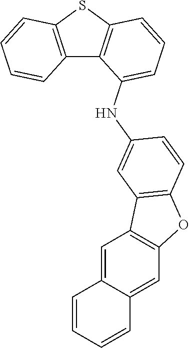 | 83% |
| 037 | 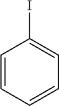 | 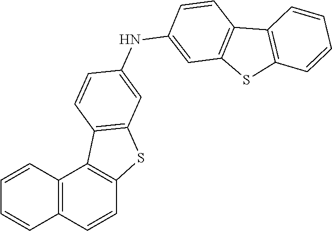 | 84% |
| 040 | 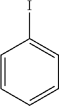 | 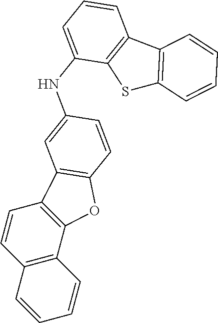 | 90% |
| 041 | 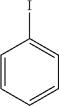 | 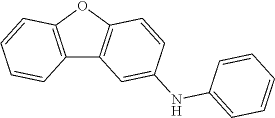 | 83% |
| 042 | 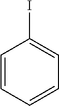 | 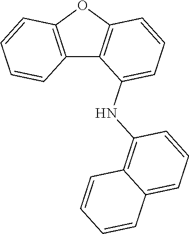 | 82% |
| 044 | 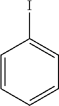 | 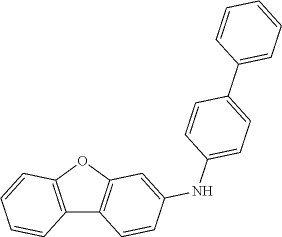 | 86% |
| 045 | 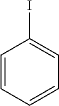 | 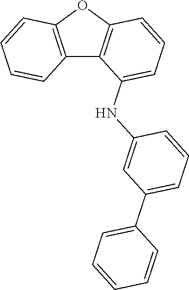 | 90% |
| 048 | 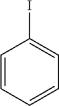 | 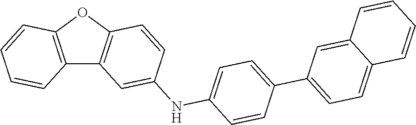 | 82% |
| 050 | 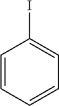 | 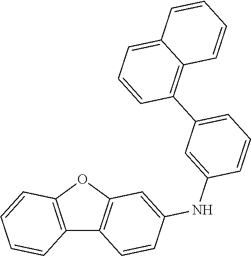 | 89% |
| 051 | 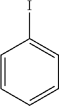 | 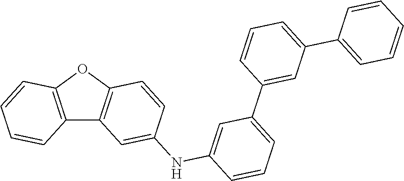 | 75% |
| 058 | 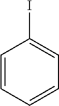 | 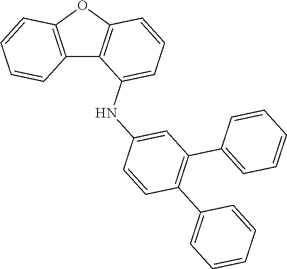 | 73% |
| 066 | 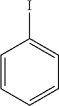 | 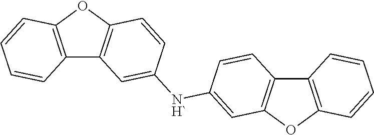 | 70% |
| 068 | 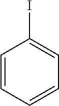 | 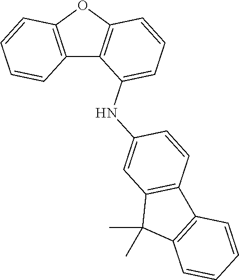 | 78% |
| 072 | 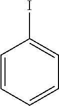 | 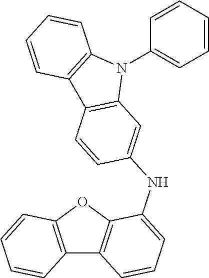 | 84% |
| 075 | 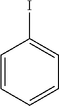 | 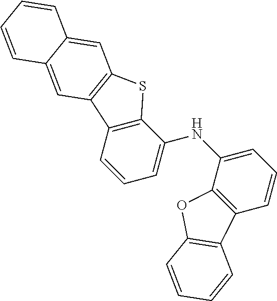 | 64% |
| 077 | 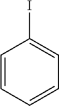 | 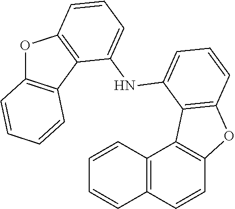 | 71% |
| 079 | 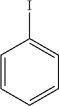 | 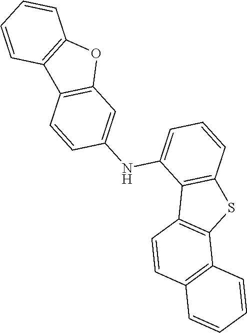 | 70% |
| 082 | 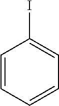 | 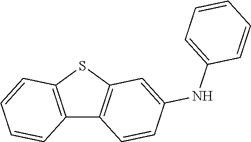 | 79% |
| 086 | 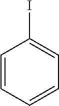 | 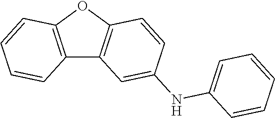 | 85% |
| 089 | 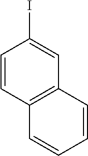 | 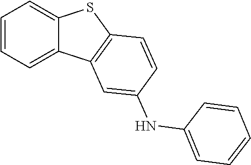 | 87% |
| 093 | 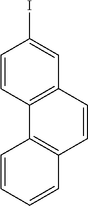 | 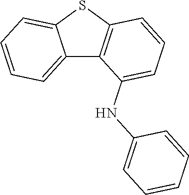 | 83% |
| 095 | 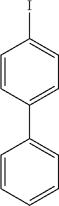 | 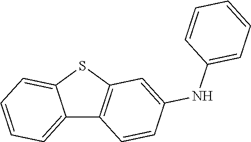 | 78% |
| 110 | 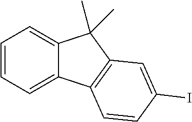 | 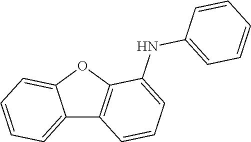 | 82% |
| 115 | 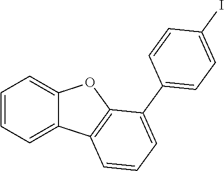 | 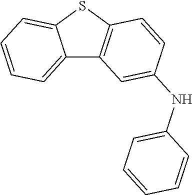 | 82% |
| 120 | 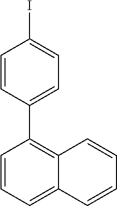 | 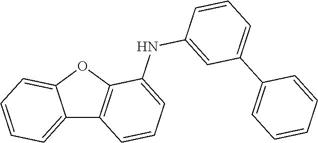 | 73% |
| 124 | 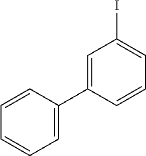 | 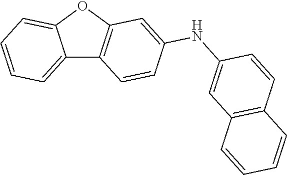 | 83% |
| 127 | 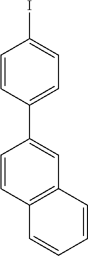 | 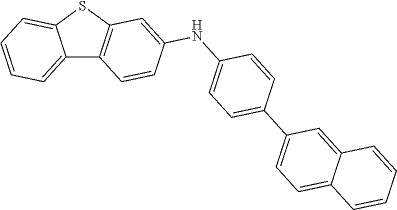 | 80% |
| 129 | 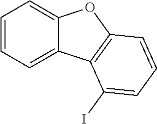 | 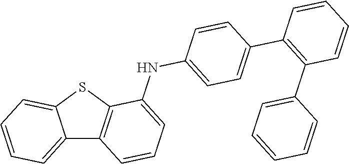 | 69% |
| 145 | 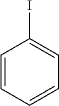 | 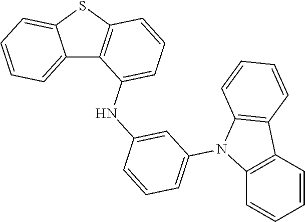 | 86% |

### [Preparation Example 2] Preparation of Compound 149

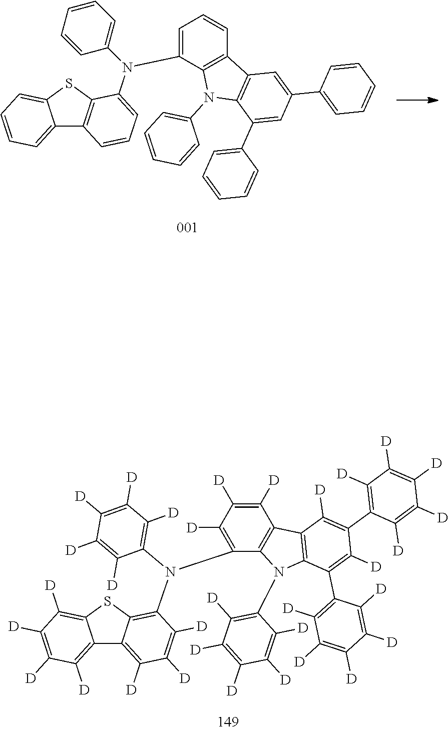 [SMILES: c1ccc(-c2cc(-c3ccccc3)c3c(c2)c2cccc(N(c4ccccc4)c4cccc5c4sc4ccccc45)c2n3-c2ccccc2)cc1 ; [2H]c1c([2H])c([2H])c(-c2c([2H])c(-c3c([2H])c([2H])c([2H])c([2H])c3[2H])c3c(c2[2H])c2c([2H])c([2H])c([2H])c(N(c4c([2H])c([2H])c([2H])c([2H])c4[2H])c4c([2H])c([2H])c([2H])c5c4sc4c([2H])c([2H])c([2H])c([2H])c45)c2n3-c2c([2H])c([2H])c([2H])c([2H])c2[2H])c([2H])c1[2H]]

Compound 001 (10 g, 14.95 mmol) was added to 50 mL of C₆D₆, and the resulting mixture was purged with nitrogen for 2 hours. 9.41 mL of trifluoromethanesulfonic acid (104.66 mmol, 7 eq) was added dropwise thereto via syringe, and the reaction mixture was heated under reflux for 2 hours. After the temperature was cooled to room temperature, 50 mL of deuterium oxide was added for extraction, and the organic layer was dried over anhydrous MgSO₄ and then concentrated using a rotary evaporator. Thereafter, the resulting product was allowed to pass through an ethylene acetate (EA) slurry to obtain Compound 149 (8 g, 76.33%).

The compounds in the following Table 2 were synthesized in the same manner as in Preparation Example 2 above, except for the reaction temperature, time, and equivalent of trifluoromethanesulfonic acid described in Preparation Example 2 above.

TABLE 2

| Compound | Reaction | Reaction | Trifluoromethanesulfonic | |
|---|---|---|---|---|
| No. | temperature | time | acid equivalent | Yield |
| 157 | RT | 1 HR | 5 eq | 68% |
| 159 | Reflux | 2 HR | 7 eq | 77% |
| 160 | Reflux | 2 HR | 3 eq | 70% |

Compounds were prepared in the same manner as in the Preparation Examples, and the synthesis confirmation results thereof are shown in Tables 3 and 4. Table 3 shows the measured values of ¹H NMR (CDCl₃, 400 MHz), and Table 4 shows the measured values of field desorption mass spectrometry (FD-MS).

TABLE 3

| Com- | |
|---|---|
| pound | |
| No. | ¹H NMR(CDCl₃, 400 MHz) |
| 1 | δ = 8.45(1H, d), 8.11(1H, d), 7.99-7.93(2H, m), 7.85(1H, |
| | d), 7.76-7.75(3H, m), 7.62-7.35(14H, m), 7.25-7.19(7H, m), |
| | 7.08-7.00(3H, m) |
| 3 | δ = 8.45(1H, d), 8.01-7.41(27H, m), 7.19-7.11(5H, m), 6.48 |
| | (1H, d) |
| 4 | δ = 8.45(1H, d), 7.99-7.93(2H, m), 7.76-7.75(5H, m), 7.62- |
| | 7.29(23H, m), 7.19(4H, m), 6.40(1H, d) |
| 5 | δ = 8.45(1H, d), 8.01-7.93(3H, m), 7.85(1H, d), 7.76- |
| | 7.75(5H, m), 7.64-7.35(18H, m), 7.27-7.17(8H, m) |
| 6 | δ = 8.45-8.39(2H, d), 8.10(1H, d), 7.99-7.93(3H, m), |
| | 7.85(1H, d), 7.76-7.75(3H, m), 7.62-7.37(17H, m), 7.19- |
| | 7.08(8H, m), 6.40(1H, d) |
| 12 | δ = 9.08(1H, d), 8.84(1H, d), 8.45(1H, d), 8.27(1H, d), |
| | 8.05-7.90(5H, m), 7.75-7.41(22H, m), 7.29-7.17(8H, m), |
| | 6.40(1H, d) |
| 16 | δ = 8.45(1H, d), 7.99-7.85(5H, m), 7.76-7.75(5H, m), |
| | 7.65-7.41(21H, m), 7.29(1H, t), 7.19(4H, m), 7.08(2H, |
| | d), 6.40(1H, d) |
| 25 | δ = 8.45(1H, d), 8.22(1H, s), 7.99-7.85(6H, m), 7.76- |
| | 7.75(3H, m), 7.62-7.19(22H, m), 6.97(1H, d) |
| 32 | δ = 8.55(1H, d), 8.45(1H, d), 8.19(1H, d), 7.99-7.93(4H, |
| | m), 7.85(1H, d), 7.76-7.16(30H, m), 6.40(1H, d) |
| 33 | δ = 8.55(1H, d), 8.45(1H, d), 8.11(1H, d), 7.99-7.93(3H, |
| | m), 7.85(1H, d), 7.76-7.75(3H, m), 7.62-7.33(23H, m), |
| | 7.25-7.16(6H, m) |
| 34 | δ = 9.08(1H, d), 8.71(1H, s), 8.45-8.33(3H, m), 8.07- |
| | 7.93(4H, m), 7.70-7.41(23H, m), 7.19-7.18(5H, m), |
| | 6.40(1H, d) |
| 36 | δ = 8.45(1H, d), 8.28-8.22(2H, m), 8.11(1H, d), 7.99- |
| | 7.93(2H, m), 7.75-7.41(23H, m), 7.29(1H, t), 7.19(4H, |
| | m), 6.97(1H, d), 6.40(1H, d) |
| 37 | δ = 8.54(1H, d), 8.45(1H, d), 8.01-7.93(5H, m), 7.85- |
| | 7.75(6H, m), 7.64-7.41(18H, m), 7.25-7.19(5H, m) |
| 40 | δ = 8.45(1H, d), 8.22-8.11(4H, m), 7.99-7.93(2H, m), |
| | 7.84(1H, d), 7.76-7.41(21H, m), 7.29(1H, t), 7.19(4H, |
| | m), 6.97(1H, d), 6.40(1H, d) |
| 41 | δ = 8.22(1H, s), 7.98(1H, d), 7.85(1H, d), 7.76-7.75(3H, |
| | m), 7.62-7.19(21H, m), 7.08-6.97(4H, m) |
| 42 | δ = 8.39(1H, d), 8.22-8.15(2H, d), 7.99-7.98(2H, m), |
| | 7.81-7.75(4H, m), 7.63-7.18(23H, m), 6.91(1H, d), |
| | 6.40(1H, d) |
| 44 | δ = 8.03-7.98(3H, m), 7.80-7.75(6H, m), 7.62-7.31(21H, |
| | m), 7.19(4H, m), 6.91(1H, d), 6.40(1H, d) |
| 45 | δ = 7.99-7.98(2H, m), 7.85(1H, d), 7.76-7.75(5H, m), |
| | 7.62-7.19(27H, m), 6.91(1H, d) |
| 48 | δ = 8.22(1H, s), 8.09-7.98(6H, m), 7.90(1H, d), 7.76- |
| | 7.75(3H, m), 7.63-7.31(21H, m), 7.19(4H, m), 6.97(1H, |
| | d), 6.48(1H, d) |
| 50 | δ = 8.95(1H, d), 8.50(1H, d), 8.39(1H, d), 8.20(1H, d), |
| | 8.09-7.98(4H, m), 7.80-7.75(4H, m), 7.62-7.18(23H, m), |
| | 6.91(1H, d), 6.40(1H, d) |
| 51 | δ = 8.22(1H, s), 8.01-7.90(5H, m), 7.76-7.73(6H, m), |
| | 7.62-7.19(26H, m), 6.97(1H, d), 6.48(1H, d) |
| 58 | δ = 8.39(1H, d), 7.99-7.92(3H, m), 7.79-7.75(7H, m), |
| | 7.62-7.18(27H, m), 6.91(1H, d), 6.40(1H, d) |
| 66 | δ = 8.39(1H, d), 8.22(1H, s), 8.03-7.98(4H, m), 7.80- |
| | 7.75(4H, m), 7.62-7.31(16H, m), 7.19-7.18(5H, m), 6.97- |
| | 6.91(2H, m), 6.40(1H, d) |
| 68 | δ = 7.99-7.98(2H, m), 7.90-7.86(2H, d), 7.76-7.75(3H, m), |
| | 7.62-7.16(25H, m), 6.91(1H, d), 6.40(1H, d) |
| 72 | δ = 7.99-7.98(2H, m), 7.90-7.86(2H, d), 7.76-7.75(3H, m), |
| | 7.62-7.16(25H, m), 6.91(1H, d), 6.40(1H, d) |
| 75 | δ = 8.28(1H, d), 8.11(2H, d), 8.01-7.98(3H, m), 7.90- |
| | 7.86(2H, m), 7.78-7.19(26H, m), 6.97(1H, d), 6.48(1H, d) |
| 77 | δ = 8.54(1H, d), 7.99-7.98(3H, m), 7.85(1H, d), 7.76- |
| | 7.75(3H, m), 7.62-7.19(26H, m), 6.91(2H, d) |
| 79 | δ = 8.16(1H, d), 8.05-7.98(6H, m), 7.90-7.75(6H, m), |
| | 7.64-7.31(17H, m), 7.19(4H, m), 6.91(1H, d), 6.48(1H, d) |
| 82 | δ = 8.45-8.39(2H, d), 8.01-7.93(3H, m), 7.76-7.75(3H, m), |
| | 7.64-7.41(13H, m), 7.24-7.18(7H, m), 7.08-7.00(3H, m), |
| | 6.40(1H, d) |
| 86 | δ = 8.39(1H, d), 8.22(1H, s), 7.99-7.98(2H, m), 7.76- |
| | 7.75(3H, m), 7.62-7.19(20H, m), 7.08-6.97(4H, m), |
| | 6.40(1H, d) |
| 89 | δ = 8.45(1H, d), 8.03-7.75(12H, m), 7.59-7.35(11H, m), |
| | 7.25-7.19(7H, m), 7.08-7.00(3H, m) |
| 93 | δ = 9.06(1H, d), 8.70(1H, d), 8.45(1H, d), 8.24(1H, d), |
| | 8.00-7.85(6H, m), 7.75-7.35(16H, m), 7.25-7.19(7H, m), |
| | 7.08-7.00(3H, m) |
| 95 | δ = 8.45(1H, d), 8.01-7.90(9H, m), 7.76-7.75(5H, m), |
| | 7.64(1H, s), 7.56-7.41(10H, m), 7.24-7.19(6H, m), 7.08- |
| | 7.00(3H, m), 6.48(1H, d) |
| 110 | δ = 8.39(1H, d), 8.04-7.98(3H, m), 7.90(1H, d), 7.76- |
| | 7.75(3H, m), 7.67-7.64(2H, m), 7.55-7.18(19H, m), 7.08- |
| | 6.97(4H, m), 6.40(1H, d), 1.69(2H, t) |
| 115 | δ = 8.45(1H, d), 8.08-7.85(13H, m), 7.76-7.75(3H, m), |
| | 7.54-7.19(17H, m), 7.08-7.00(3H, m), 6.48(1H, d) |
| 120 | δ = 8.95(1H, d), 8.50(1H, d), 8.20(1H, d), 8.09(1H, d), |
| | 7.99-7.91(6H, m), 7.77-7.75(6H, m), 7.64-7.17(24H, m), |
| | 6.97(1H, d), 6.40(1H, d) |
| 124 | δ = 8.21(1H, s), 8.03-7.98(3H, m), 7.80-7.31(22H, m), |
| | 7.19(4H, m), 7.11(1H, s), 6.91(1H, d), 6.40(1H, d) |
| 127 | δ = 8.45(1H, d), 8.11-7.90(11H, m), 7.76-7.75(5H, m), |
| | 7.64-7.37(20H, m), 7.19(8H, m), 6.48(1H, d) |
| 129 | δ = 8.45(1H, d), 8.11(1H, d), 7.99-7.93(5H, m), 7.85- |
| | 7.75(6H, m), 7.60-7.19(29H, m) |
| 145 | δ = 8.55(1H, d), 8.45(1H, d), 8.19(1H, d), 7.99-7.93(3H, |
| | m), 7.85(1H, d), 7.76-7.75(3H, m), 7.62-7.35(20H, m), |
| | 7.25-7.16(9H, m), 7.04(1H, d) |
| 149 | All D-substituent |
| 157 | All D-substituent |
| 159 | All D-substituent |
| 160 | All D-substituent |

TABLE 4

| Compound | | Compound | |
|---|---|---|---|
| No. | FD-MS | No. | FD-MS |
| 1 | m/z = 668.86 | 3 | m/z = 718.92 |
| | (C48H32N2S = 668.23) | | (C52H34N2S = 718.24) |
| 4 | m/z = 744.96 | 5 | m/z = 744.96 |
| | (C54H36N2S = 744.26) | | (C54H36N2S = 744.26) |
| 6 | m/z = 744.96 | 12 | m/z = 845.08 |
| | (C54H36N2S = 744.26) | | (C62H40N2S = 844.29) |
| 16 | m/z = 821.05 | 25 | m/z = 758.94 |
| | (C60H40N2S = 820.29) | | (C54H34N2OS = 758.24) |
| 32 | m/z = 834.05 | 33 | m/z = 834.05 |
| | (C60H39N3S = 833.29) | | (C60H39N3S = 833.29) |
| 34 | m/z = 819.04 | 36 | m/z = 809.00 |
| | (C60H38N2S = 818.28) | | (C58H36N2OS = 808.25) |
| 37 | m/z = 825.06 | 40 | m/z = 809.00 |
| | (C58H36N2S2 = 824.23) | | (C58H36N2OS = 808.25) |
| 41 | m/z = 652.80 | 42 | m/z = 702.86 |
| | (C48H32N2O = 652.25) | | (C52H34N2O = 702.27) |
| 44 | m/z = 728.89 | 45 | m/z = 728.89 |
| | (C54H36N2O = 728.28) | | (C54H36N2O = 728.28) |
| 48 | m/z = 778.95 | 50 | m/z = 778.95 |
| | (C58H38N2O = 778.30) | | (C58H38N2O = 778.30) |
| 51 | m/z = 804.99 | 58 | m/z = 804.99 |
| | (C60H40N2O = 804.31) | | (C60H40N2O = 804.31) |
| 66 | m/z = 742.88 | 68 | m/z = 768.96 |
| | (C54H34N2O2 = 742.26) | | (C57H40N2O = 768.31) |
| 72 | m/z = 817.99 | 75 | m/z = 809.00 |
| | (C60H39N3O = 817.31) | | (C58H36N2OS = 808.25) |
| 77 | m/z = 792.94 | 79 | m/z = 809.00 |
| | (C58H36N2O2 = 792.28) | | (C58H36N2OS = 808.25) |
| 82 | m/z = 668.86 | 86 | m/z = 652.80 |
| | (C48H32N2S = 668.23) | | (C48H32N2O = 652.25) |
| 89 | m/z = 718.92 | 93 | m/z = 768.98 |
| | (C52H34N2S = 718.24) | | (C56H36N2S = 768.26) |
| 95 | m/z = 744.96 | 110 | m/z = 768.96 |
| | (C54H36N2S = 744.26) | | (C57H40N2O = 768.31) |
| 115 | m/z = 835.04 | 120 | m/z = 855.05 |
| | (C60H38N2OS = 834.27) | | (C64H42N2O = 854.33) |
| 124 | m/z = 778.95 | 127 | m/z = 947.21 |
| | (C58H38N2O = 778.30) | | (C70H46N2S = 946.34) |
| 129 | m/z = 911.13 | 145 | m/z = 834.05 |
| | (C66H42N2OS = 910.30) | | (C60H39N3S = 833.29) |
| 149 | m/z = 701.05 | 157 | m/z = 873.29 |
| | (C48D32N2S = 700.43) | | (C60D39N3S = 872.53) |
| 159 | m/z = 684.99 | 160 | m/z = 765.11 |
| | (C48D32N2O = 684.45) | | (C54D36N2O = 764.51) |

### EXPERIMENTAL EXAMPLES

### Experimental Example 1

### 1) Manufacture of Organic Light Emitting Device

### Comparative Example 1

Trichloroethylene, acetone, ethanol, and distilled water were each sequentially used to ultrasonically wash a transparent electrode ITO thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

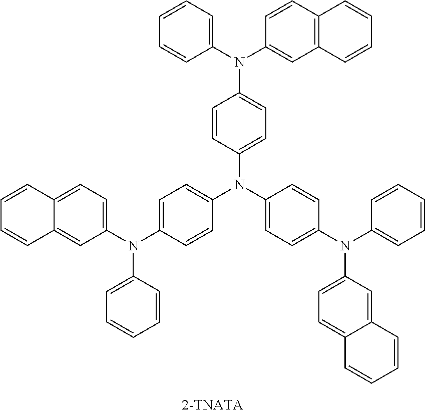 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)-N,N′-diphenyl-4,4′-diamine (NPB) in another cell in the vacuum deposition equipment and applying current to the cell to evaporate NPB.

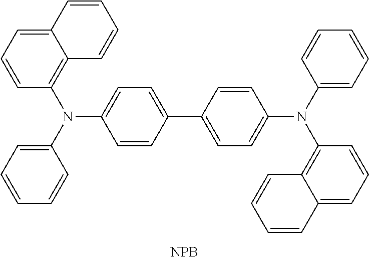 [SMILES: c1ccc(N(c2ccc(-c3ccc(N(c4ccccc4)c4cccc5ccccc45)cc3)cc2)c2cccc3ccccc23)cc1 ; "NPB"]

The hole injection layer and the hole transport layer were formed as described above, and then a blue light emitting material having the following structure as an emission layer was deposited thereon. Specifically, a blue light emitting host material H1 was vacuum deposited to have a thickness of 200 Å on one cell in the vacuum deposition apparatus, and a blue light emitting dopant material D1 was vacuum deposited thereon in an amount of 5% based on the host material.

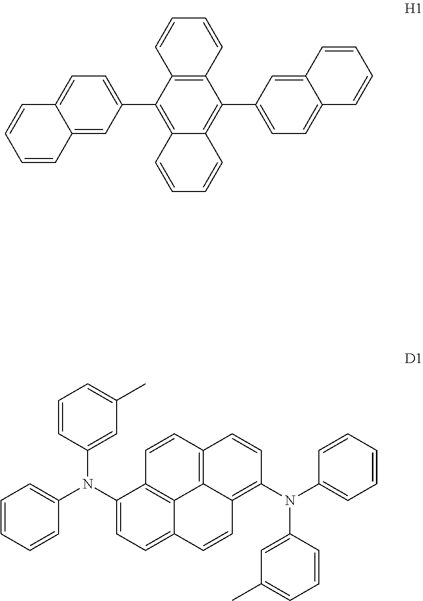

Subsequently, a compound having the following structural formula E1 as an electron transport layer was deposited to have a thickness of 300 Å.

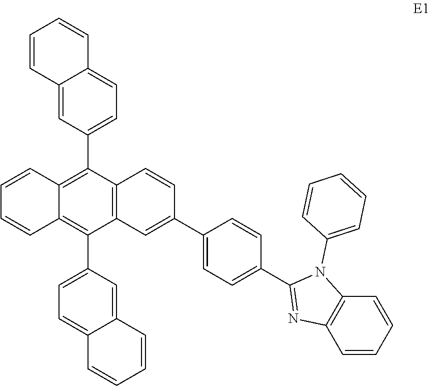 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å.

Meanwhile, all the organic compounds required for manufacturing an OLED device were subjected to vacuum sublimed purification under 10⁻⁸ to 10⁻⁶ torr for each material, and used for the manufacture of the OLED.

### Comparative Examples 2 to 7 and Examples 1 to 42

An organic light emitting device was manufactured in the same manner as in Comparative Example 1, except that the heterocyclic compound in the following Table 5 was used instead of NPB used when a hole transport layer was formed in Experimental Example 1.

### 2) Evaluation of Organic Light Emitting Device

For the organic light emitting device manufactured as described above, the electroluminescence (EL) characteristics were measured using M7000 manufactured by McScience Inc., and the measurement results were used to measure T₉₅ through a lifetime measurement device (M6000) manufactured by McScience Inc., when the reference luminance was 700 cd/m². The results of measuring the driving voltage, light emitting efficiency, color coordinate (CIE), and service life of the blue organic light emitting device manufactured according to the present invention are shown in the following Table 5.

TABLE 5

| | Hetero ring | | Light | | |
|---|---|---|---|---|---|
| | Compound | Driving | emitting | | Service |
| | (Hole | voltage | efficiency | CIE | life |
| No. | transport) | (V) | (cd/A) | (x, y) | (T₉₅) |
| Example 1 | 001 | 4.80 | 6.81 | (0.133, 0.100) | 90 |
| Example 2 | 003 | 4.84 | 6.78 | (0.134, 0.100) | 86 |
| Example 3 | 004 | 4.85 | 6.80 | (0.134, 0.100) | 89 |
| Example 4 | 005 | 4.80 | 6.83 | (0.134, 0.101) | 88 |
| Example 5 | 006 | 4.87 | 6.73 | (0.133, 0.101) | 85 |
| Example 6 | 012 | 4.81 | 6.85 | (0.134, 0.100) | 90 |
| Example 7 | 016 | 4.85 | 6.90 | (0.133, 0.101) | 89 |
| Example 8 | 025 | 4.90 | 6.74 | (0.133, 0.102) | 86 |
| Example 9 | 032 | 4.87 | 6.88 | (0.134, 0.101) | 89 |
| Example 10 | 033 | 4.86 | 6.80 | (0.134, 0.101) | 89 |
| Example 11 | 034 | 4.92 | 6.70 | (0.133, 0.100) | 92 |
| Example 12 | 036 | 4.88 | 6.85 | (0.134, 0.100) | 87 |
| Example 13 | 037 | 4.82 | 6.85 | (0.134, 0.100) | 85 |
| Example 14 | 040 | 4.83 | 6.84 | (0.133, 0.100) | 85 |
| Example 15 | 041 | 4.81 | 6.86 | (0.134, 0.101) | 88 |
| Example 16 | 042 | 4.85 | 6.87 | (0.134, 0.101) | 84 |
| Example 17 | 044 | 4.89 | 6.88 | (0.133, 0.100) | 90 |
| Example 18 | 045 | 4.83 | 6.85 | (0.133, 0.101) | 88 |
| Example 19 | 048 | 4.86 | 6.87 | (0.134, 0.100) | 85 |
| Example 20 | 050 | 4.91 | 6.71 | (0.134, 0.100) | 86 |
| Example 21 | 051 | 4.85 | 6.90 | (0.133, 0.101) | 85 |
| Example 22 | 058 | 4.86 | 6.88 | (0.134, 0.101) | 84 |
| Example 23 | 066 | 4.81 | 6.93 | (0.133, 0.101) | 86 |
| Example 24 | 068 | 4.91 | 6.95 | (0.134, 0.100) | 95 |
| Example 25 | 072 | 4.83 | 6.88 | (0.133, 0.101) | 87 |
| Example 26 | 075 | 4.84 | 6.90 | (0.133, 0.100) | 91 |
| Example 27 | 077 | 4.90 | 6.85 | (0.134, 0.100) | 88 |
| Example 28 | 079 | 4.85 | 6.83 | (0.134, 0.100) | 87 |
| Example 29 | 082 | 4.91 | 6.81 | (0.134, 0.100) | 89 |
| Example 30 | 086 | 4.85 | 6.90 | (0.134, 0.100) | 90 |
| Example 31 | 089 | 4.84 | 6.88 | (0.133, 0.101) | 90 |
| Example 32 | 093 | 4.80 | 6.90 | (0.133, 0.100) | 91 |
| Example 33 | 095 | 4.87 | 6.87 | (0.133, 0.100) | 87 |
| Example 34 | 110 | 4.87 | 6.85 | (0.134, 0.101) | 85 |
| Example 35 | 115 | 4.86 | 6.87 | (0.133, 0.101) | 88 |
| Example 36 | 120 | 4.92 | 6.71 | (0.134, 0.100) | 91 |
| Example 37 | 124 | 4.88 | 6.90 | (0.134, 0.100) | 88 |
| Example 38 | 127 | 4.82 | 6.88 | (0.134, 0.100) | 85 |
| Example 39 | 129 | 4.83 | 6.93 | (0.133, 0.101) | 89 |
| Example 40 | 142 | 4.81 | 6.85 | (0.134, 0.101) | 90 |
| Example 41 | 149 | 4.89 | 6.78 | (0.134, 0.100) | 94 |
| Example 42 | 157 | 4.85 | 6.70 | (0.134, 0.100) | 93 |
| Comparative | NPB | 5.54 | 6.05 | (0.134, 0.100) | 61 |
| Example : | | | | | |
| Comparative | Comparative | 5.39 | 6.28 | (0.133, 0.100) | 64 |
| Example 2 | Compound A | | | | |
| Comparative | Comparative | 5.44 | 6.19 | (0.133, 0.101) | 60 |
| Example 3 | Compound B | | | | |
| Comparative | Comparative | 5.42 | 6.20 | (0.134, 0.100) | 68 |
| Example 4 | Compound C | | | | |
| Comparative | Comparative | 5.50 | 6.21 | (0.133, 0.100) | 61 |
| Example 5 | Compound D | | | | |
| Comparative | Comparative | 5.48 | 6.25 | (0.133, 0.101) | 72 |
| Example 6 | Compound E | | | | |
| Comparative | Comparative | 5.41 | 6.17 | (0.133, 0.100) | 62 |
| Example 7 | Compound F | | | | |

The structures of Comparative Compounds A to F are as follows.

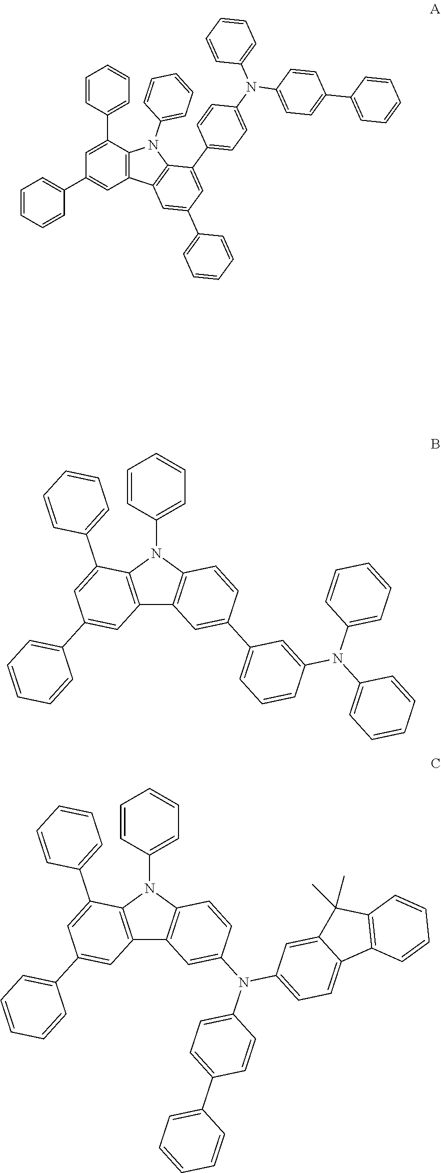 [SMILES: c1ccc(-c2ccc(N(c3ccccc3)c3ccc(-c4cc(-c5ccccc5)cc5c6cc(-c7ccccc7)cc(-c7ccccc7)c6n(-c6ccccc6)c45)cc3)cc2)cc1 ; c1ccc(-c2cc(-c3ccccc3)c3c(c2)c2cc(-c4cccc(N(c5ccccc5)c5ccccc5)c4)ccc2n3-c2ccccc2)cc1 ; CC1(C)c2ccccc2-c2ccc(N(c3ccc(-c4ccccc4)cc3)c3ccc4c(c3)c3cc(-c5ccccc5)cc(-c5ccccc5)c3n4-c3ccccc3)cc21]

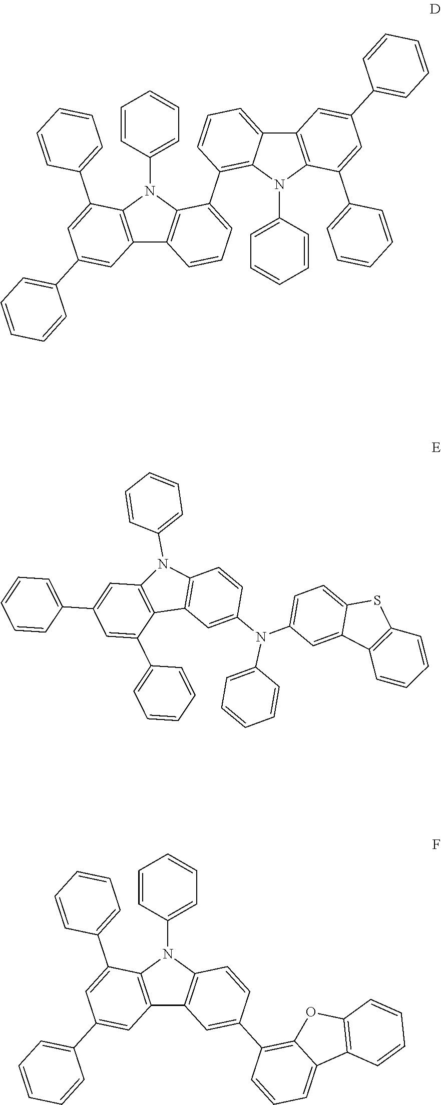 [SMILES: c1ccc(-c2cc(-c3ccccc3)c3c(c2)c2cccc(-c4cccc5c6cc(-c7ccccc7)cc(-c7ccccc7)c6n(-c6ccccc6)c45)c2n3-c2ccccc2)cc1 ; c1ccc(-c2cc(-c3ccccc3)c3c4cc(N(c5ccccc5)c5ccc6sc7ccccc7c6c5)ccc4n(-c4ccccc4)c3c2)cc1 ; c1ccc(-c2cc(-c3ccccc3)c3c(c2)c2cc(-c4cccc5c4oc4ccccc45)ccc2n3-c2ccccc2)cc1]

As can be seen from the results in Table 5, the organic light emitting device using the heterocyclic compound of the present invention as a hole transport material has a low driving voltage and remarkably improved light emitting efficiency and service life compared to Comparative Examples 1 to 7. In particular, it could be confirmed that the service life of the organic light emitting devices of Examples 1 to 42 was remarkably improved by 16.6% or more compared to that of Comparative Examples 1 to 7.

The reason for this result is that Compound A used in Comparative Example 2 has an amine structure different from that of the present invention and does not have a substituent including a heteroatom (O or S), and four phenyl groups are substituted based on the benzene ring at both ends of the carbazole group, which makes the hole characteristics higher than required, and for this reason, it is impossible to implement the hole mobility suitable for the device proposed in the present invention. Unlike Comparative Example 6 in which the substituents are located at positions 2 and 4, the present invention has a skeleton in which the substituents are located at positions 1 and 3, and has an advantage of exhibiting suitable physical properties as a hole transport layer by easily implementing a more suitable hole mobility than when used as a material for the organic light emitting device of the present invention.

Further, Comparative Examples 3 and 4 are different from the heterocyclic compound of the present invention, which includes a dibenzofuranyl group and/or a dibenzothiophenyl group as a substituent, in the type of substituent, and in addition, Comparative Example 5 has a symmetrical biscarbazole structure, but is not an amine, Comparative Example 6 has the positions of two phenyl groups substituted on the carbazole, which are out of the structure of the present invention, and Comparative Example 7 is different from the heterocyclic compound of the present invention in that a dibenzofuranyl group and/or a dibenzothiophenyl group are/is directly bonded to the benzene ring on the other side of the carbazole in which two phenyls are substituted, and thus the compound is not an amine. The heterocyclic compound specified in the present invention may implement hole mobility at a rate suitable for a device by the suitable bond length and strength of the first to third substituents to efficiently transport holes by forming a more stable compound without decomposition or destruction of the compound. Furthermore, when the compound of Comparative Example 5 is used to form a film on a device, the film forming characteristics deteriorate, so that there was a disadvantage in that the device characteristics may remarkably deteriorate because it is difficult to form a uniform film.

Therefore, it is determined that the heterocyclic compound of the present invention enhances electron-transporting characteristics or stability and is therefore excellent in all device characteristics such as driving, efficiency, and service life.

### Experimental Example 2

### 1) Manufacture of Organic Light Emitting Device

### Comparative Example 8

Trichloroethylene, acetone, ethanol, and distilled water were each sequentially used to ultrasonically wash a transparent electrode ITO thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

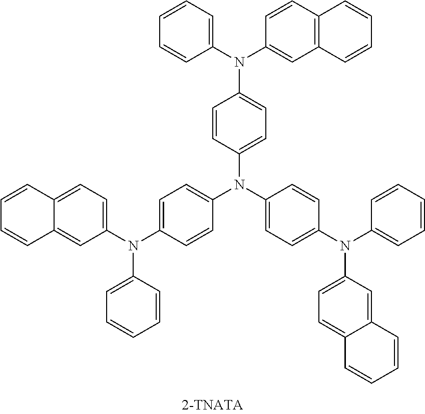 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 250 Å was deposited on the hole injection layer by placing the following N,N′-bis(α-naphthyl)-N,N′-diphenyl-4,4′-diamine (NPB) in another cell in the vacuum deposition equipment and applying current to the cell to evaporate NPB. Subsequently, an electron blocking layer having a thickness of 50 Å was formed on top of the hole transport layer using 1,3,5-tris(3-pyridyl-3-phenyl)benzene (TmPyPB).

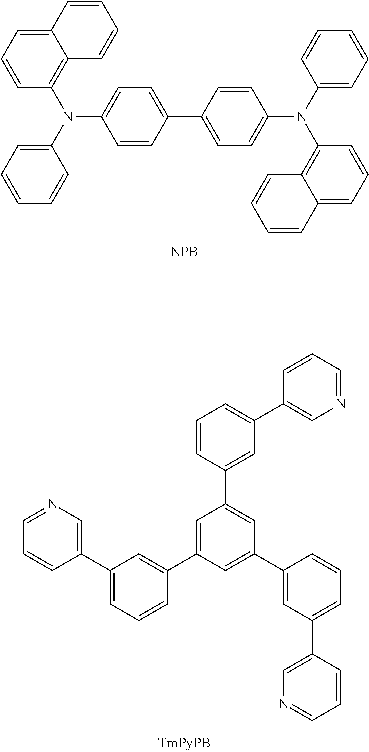 [SMILES: c1ccc(N(c2ccc(-c3ccc(N(c4ccccc4)c4cccc5ccccc45)cc3)cc2)c2cccc3ccccc23)cc1 ; "NPB" ; c1cncc(-c2cccc(-c3cc(-c4cccc(-c5cccnc5)c4)cc(-c4cccc(-c5cccnc5)c4)c3)c2)c1]

The electron blocking layer was formed as described above, and then a blue light emitting material having the following structure as an emission layer was deposited thereon. Specifically, a blue light emitting host material H1 was vacuum deposited to have a thickness of 200 Å on one cell in the vacuum deposition apparatus, and a blue light emitting dopant material D1 was vacuum deposited thereon in an amount of 5% based on the host material.

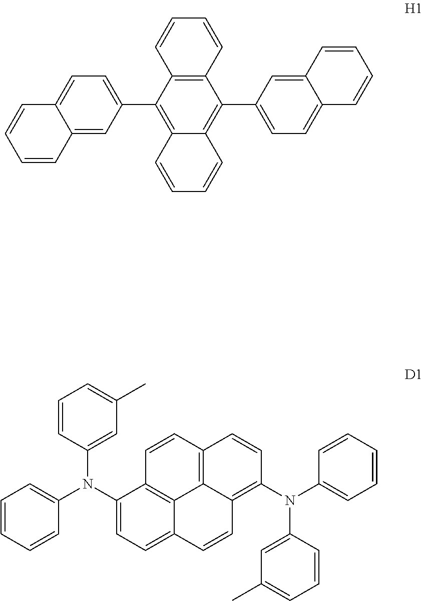

Subsequently, a compound having the following structural formula E1 as an electron transport layer was deposited to have a thickness of 300 Å.

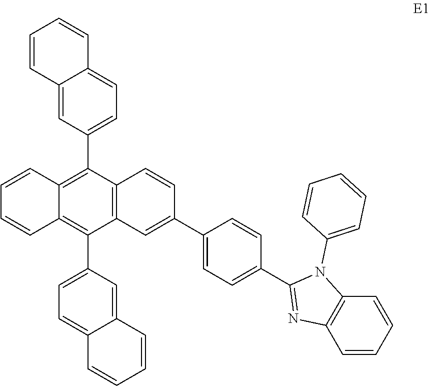 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10⁻⁸ to 10⁻⁶ torr for each material, and used for the manufacture of OLED.

### Comparative Examples 9 to 14 and Examples 43 to 84

An organic light emitting device was manufactured in the same manner as in Comparative Example 8, except that in Experimental Example 2, an electron blocking layer having a thickness of 50 Å was formed using a heterocyclic compound shown in the following Table 6 instead of an electron blocking layer having a thickness of 50 Å and including TmPyPB.

### 2) Evaluation of Organic Light Emitting Device

For the organic light emitting device manufactured as described above, the electroluminescence (EL) characteristics were measured using M7000 manufactured by McScience Inc., and the measurement results were used to measure T₉₅ through a lifetime measurement device (M6000) manufactured by McScience Inc., when the reference luminance was 700 cd/m².

The results of measuring the driving voltage, light emitting efficiency, color coordinate (CIE) and service life of the blue organic light emitting device manufactured according to the present invention are shown in the following Table 6.

TABLE 6

| | Hetero ring | | Light | | |
|---|---|---|---|---|---|
| | Compound | Driving | emitting | | Service |
| | (Electron | voltage | efficiency | CIE | life |
| No. | blocking) | (V) | (cd/A) | (x, y) | (T₉₅) |
| Example 43 | 001 | 7.31 | 68.53 | (0.215, 0.419) | 86 |
| Example 44 | 003 | 7.40 | 70.03 | (0.216, 0,424) | 90 |
| Example 45 | 004 | 7.34 | 69.87 | (0.214, 0.425) | 85 |
| Example 46 | 005 | 7.22 | 68.13 | (0.214, 0.418) | 86 |
| Example 47 | 006 | 7.32 | 69.00 | (0.214, 0.418) | 85 |
| Example 48 | 012 | 7.30 | 69.50 | (0.215, 0.417) | 84 |
| Example 49 | 016 | 7.22 | 67.72 | (0.209, 0.422) | 89 |
| Example 50 | 025 | 7.33 | 68.58 | (0.210, 0.428) | 86 |
| Example 51 | 032 | 7.35 | 69.13 | (0.209, 0.428) | 87 |
| Example 52 | 033 | 7.31 | 68.43 | (0.210, 0.430) | 91 |
| Example 53 | 034 | 7.38 | 69.84 | (0.212, 0.428) | 83 |
| Example 54 | 036 | 7.32 | 70.18 | (0.215, 0.424) | 90 |
| Example 55 | 037 | 7.29 | 70.02 | (0.214, 0.422) | 84 |
| Example 56 | 040 | 7.30 | 70.42 | (0.215, 0.417) | 88 |
| Example 57 | 041 | 7.27 | 70.25 | (0.214, 0.420) | 85 |
| Example 58 | 042 | 7.33 | 69.39 | (0.214, 0.418) | 86 |
| Example 59 | 044 | 7.21 | 69.88 | (0.214, 0.418) | 95 |
| Example 60 | 045 | 7.31 | 69.98 | (0.215, 0.419) | 91 |
| Example 61 | 048 | 7.25 | 68.80 | (0.214, 0.426) | 90 |
| Example 62 | 050 | 7.30 | 69.22 | (0.215, 0.424) | 85 |
| Example 63 | 051 | 7.28 | 70.13 | (0.219, 0.422) | 87 |
| Example 64 | 058 | 7.03 | 67.58 | (0.214, 0.418) | 88 |
| Example 65 | 066 | 7.20 | 69.77 | (0.213, 0.417) | 84 |
| Example 66 | 068 | 7.34 | 68.68 | (0.213, 0.420) | 85 |
| Example 67 | 072 | 7.32 | 69.12 | (0.215, 0.420) | 86 |
| Example 68 | 075 | 7.25 | 69.15 | (0.215, 0.420) | 88 |
| Example 69 | 077 | 7.24 | 68.88 | (0.214, 0.420) | 85 |
| Example 70 | 079 | 7.33 | 69.02 | (0.214, 0.418) | 87 |
| Example 71 | 082 | 7.05 | 68.80 | (0.215, 0.420) | 87 |
| Example 72 | 086 | 7.30 | 69.22 | (0.215, 0.421) | 91 |
| Example 73 | 089 | 7.28 | 70.13 | (0.219, 0.422) | 90 |
| Example 74 | 093 | 7.03 | 67.58 | (0.214, 0.418) | 86 |
| Example 75 | 095 | 7.27 | 69.77 | (0.213, 0.417) | 89 |
| Example 76 | 110 | 7.26 | 68.80 | (0.213, 0.420) | 90 |
| Example 77 | 115 | 7.33 | 69.22 | (0.214, 0.420) | 86 |
| Example 78 | 120 | 7.32 | 67.71 | (0.211, 0.430) | 85 |
| Example 79 | 124 | 7.25 | 66.23 | (0.213, 0.426) | 90 |
| Example 80 | 127 | 7.24 | 68.31 | (0.215, 0.420) | 87 |
| Example 81 | 129 | 7.33 | 68.61 | (0.214, 0.420) | 91 |
| Example 82 | 142 | 7.05 | 70.10 | (0.214, 0.418) | 86 |
| Example 83 | 159 | 7.30 | 67.83 | (0.214, 0.420) | 93 |
| Example 84 | 160 | 7.28 | 66.88 | (0.215, 0.420) | 95 |
| Comparative | TmPyPB | 8.20 | 57.71 | (0.211, 0.430) | 85 |
| Example 8 | | | | | |
| Comparative | Comparative | 7.86 | 56.23 | (0.213, 0.426) | 60 |
| Example 9 | Compound A | | | | |
| Comparative | Comparative | 7.79 | 57.31 | (0.209, 0.425) | 64 |
| Example 10 | Compound B | | | | |
| Comparative | Comparative | 7.71 | 58.61 | (0.211, 0.428) | 63 |
| Example 11 | Compound C | | | | |
| Comparative | Comparative | 7.80 | 57.10 | (0.209, 0.424) | 50 |
| Example 12 | Compound D | | | | |
| Comparative | Comparative | 7.67 | 57.83 | (0.212, 0.426) | 66 |
| Example 13 | Compound E | | | | |
| Comparative | Comparative | 7.84 | 56.88 | (0.209, 0.425) | 65 |
| Example 14 | Compound F | | | | |

The structures of Comparative Compounds A to F are as follows.

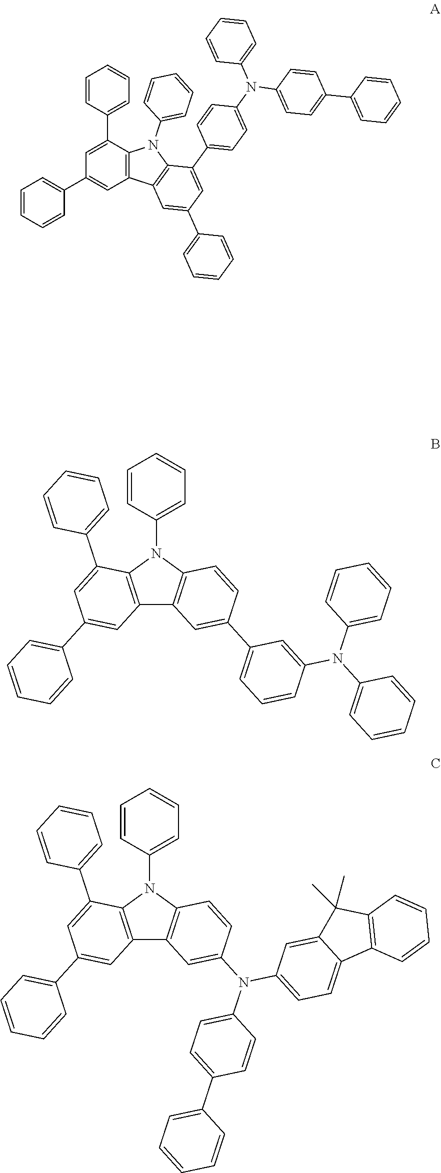 [SMILES: c1ccc(-c2ccc(N(c3ccccc3)c3ccc(-c4cc(-c5ccccc5)cc5c6cc(-c7ccccc7)cc(-c7ccccc7)c6n(-c6ccccc6)c45)cc3)cc2)cc1 ; c1ccc(-c2cc(-c3ccccc3)c3c(c2)c2cc(-c4cccc(N(c5ccccc5)c5ccccc5)c4)ccc2n3-c2ccccc2)cc1 ; CC1(C)c2ccccc2-c2ccc(N(c3ccc(-c4ccccc4)cc3)c3ccc4c(c3)c3cc(-c5ccccc5)cc(-c5ccccc5)c3n4-c3ccccc3)cc21]

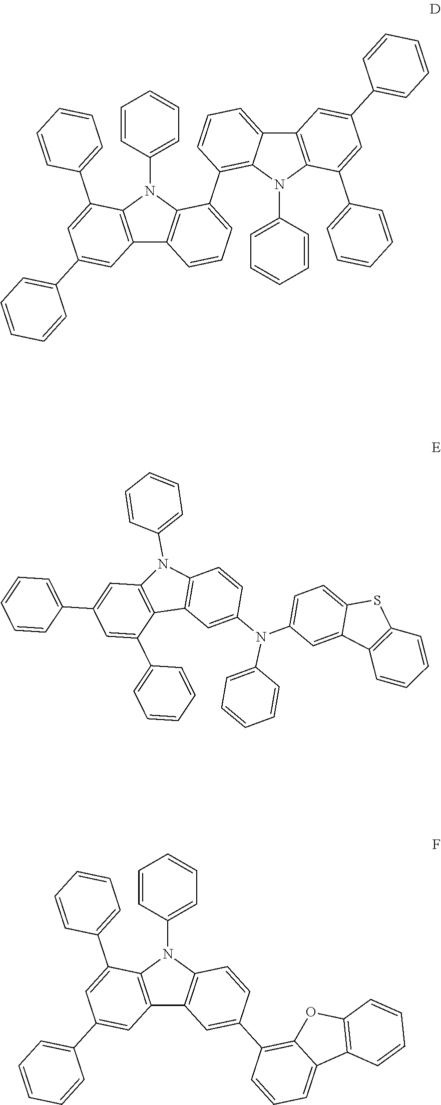 [SMILES: c1ccc(-c2cc(-c3ccccc3)c3c(c2)c2cccc(-c4cccc5c6cc(-c7ccccc7)cc(-c7ccccc7)c6n(-c6ccccc6)c45)c2n3-c2ccccc2)cc1 ; c1ccc(-c2cc(-c3ccccc3)c3c4cc(N(c5ccccc5)c5ccc6sc7ccccc7c6c5)ccc4n(-c4ccccc4)c3c2)cc1 ; c1ccc(-c2cc(-c3ccccc3)c3c(c2)c2cc(-c4cccc5c4oc4ccccc45)ccc2n3-c2ccccc2)cc1]

As can be seen from the results in Table 6, the organic light emitting device using an electron blocking material for the blue organic light emitting device of the present invention had a lower driving voltage and improved service life and light emitting efficiency compared to Comparative Examples 8 to 14. In particular, it could be confirmed that the service life of the organic light emitting devices of Examples 43 to 84 was remarkably improved by 27.7% or more compared to that of Comparative Examples 8 to 14.

Further, unlike Comparative Example 9, which has a 4-substituted structure based on the benzene ring of the carbazole group, the heterocyclic compound of the present invention has a 3-substituted structure based on the benzene ring of the carbazole group, and unlike Comparative Example 13, which has a skeleton in which the substituents are located at positions 2 and 4, the compound of the present invention has a skeleton in which the substituents are located at positions 1 and 3, and thus has an advantage in that the LUMO level can be adjusted to a level more suitable for an electron blocking layer in the device proposed in the present invention.

In addition, Comparative Example 12 has a symmetric structure, and has a disadvantage in that the device characteristics may remarkably deteriorate because the crystallinity is too high to easily form a uniform film when an electron blocking layer is prepared. Although Comparative Examples 10, 11, and 14 have an asymmetric structure, the electron and hole mobility suitable for the device used in the present invention could not be implemented compared to the heterocyclic compound specified in the present invention and a compound to which a substituent such as an amine group and an aryl group is bonded.

Furthermore, when electrons are not bonded in the emission layer and pass through the hole transport layer to the positive electrode, there is a problem in that the efficiency and service life of the OLED device are reduced. When a compound with high LUMO and T1 levels is used as an electron blocking layer in order to prevent this phenomenon, the probability that electrons passing through the emission layer to the positive electrode form excitons in the emission layer is increased. From these results, it is determined that the heterocyclic compound of the present invention provides excellence in all aspects of driving, efficiency, and service life.

The present invention is not limited to the Examples, but may be prepared in various forms, and a person with ordinary skill in the art to which the present invention pertains will understand that the present invention can be implemented in another specific form without changing the technical spirit or essential feature of the present invention. Therefore, it should be understood that the above-described Examples are illustrative only in all aspects and are not restrictive.

What is claimed is:

1. A heterocyclic compound represented by the following Chemical Formula 1: 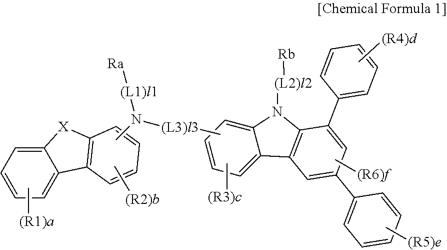
wherein, in Chemical Formula 1,
X is O; or S,
Ra and Rb are the same as or different from each other, and are each independently a substituted or unsubstituted C6 to C60 aryl group; or a substituted or unsubstituted C2 to C60 heteroaryl group,
L1 to L3 are the same as or different from each other, and are each independently a direct bond; or a substituted or unsubstituted C6 to C60 arylene group,
each of l1 to l3 is an integer from 0 to 4, and
when each of l1 to l3 is an integer of 2 or higher, substituents in the parenthesis are the same as or different from each other,
R1 to R6 are the same as or different from each other, and are each independently hydrogen; deuterium; a cyano group; a halogen group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted silyl group; or a substituted or unsubstituted phosphine oxide group,
a is an integer from 0 to 4,
each of b and c is an integer from 0 to 3,
each of d and e is an integer from 0 to 5,
f is an integer from 0 to 2, and
when each of a, b, c, d, e, and f is 2 or an integer higher than 2, substituents in the parenthesis are the same as or different from each other.

2. The heterocyclic compound of claim 1, wherein Chemical Formula 1 is represented by any one of the following Chemical Formulae 1-1 to 1-4: 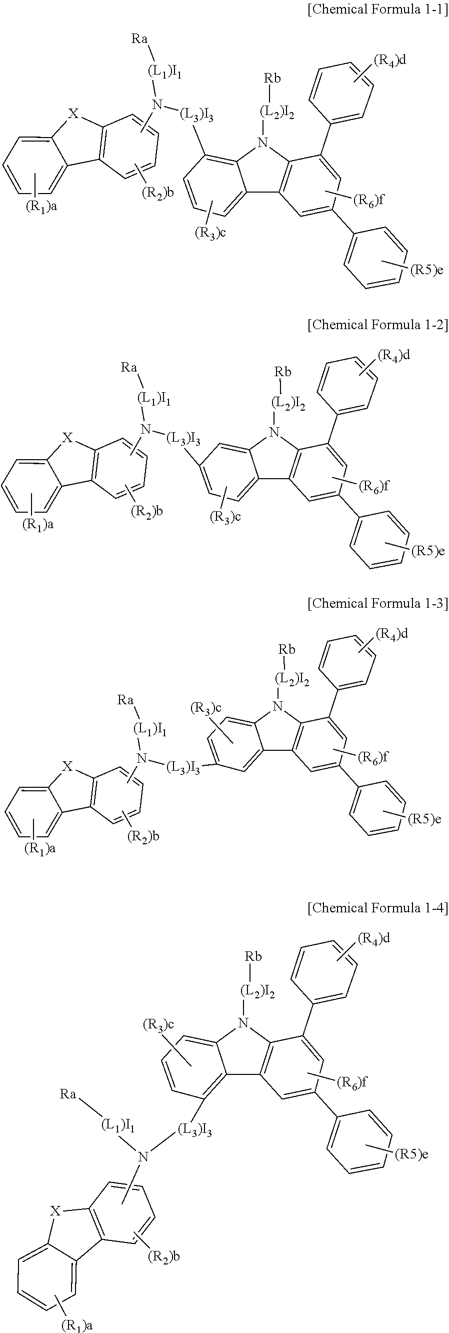
in Chemical Formula 1-1 to 1-4,
each of X, Ra, Rb, L1 to L3, l1 to l3, R1 to R6, a, b, c, d, e, and f is the same as that defined in Chemical Formula 1.

3. The heterocyclic compound of claim 1, wherein Ra and Rb are the same as or different from each other, and are each independently a substituted or unsubstituted C6 to C30 aryl group; or a C2 to C30 heteroaryl group comprising at least one heteroatom from N, O, and S and substituted or unsubstituted.

4. The heterocyclic compound of claim 1, wherein L1 to L3 are the same as or different from each other, and are each independently a direct bond; or a substituted or unsubstituted C6 to C30 arylene group.

5. The heterocyclic compound of claim 1, wherein R1 to R6 are the same as or different from each other, and are each independently hydrogen; or deuterium.

6. The heterocyclic compound of claim 1, wherein a deuterium content of the heterocyclic compound represented by Chemical Formula 1 is 0%, or 1% to 100%.

7. The heterocyclic compound of claim 1, wherein Chemical Formula 1 is represented by any one of the following compounds:

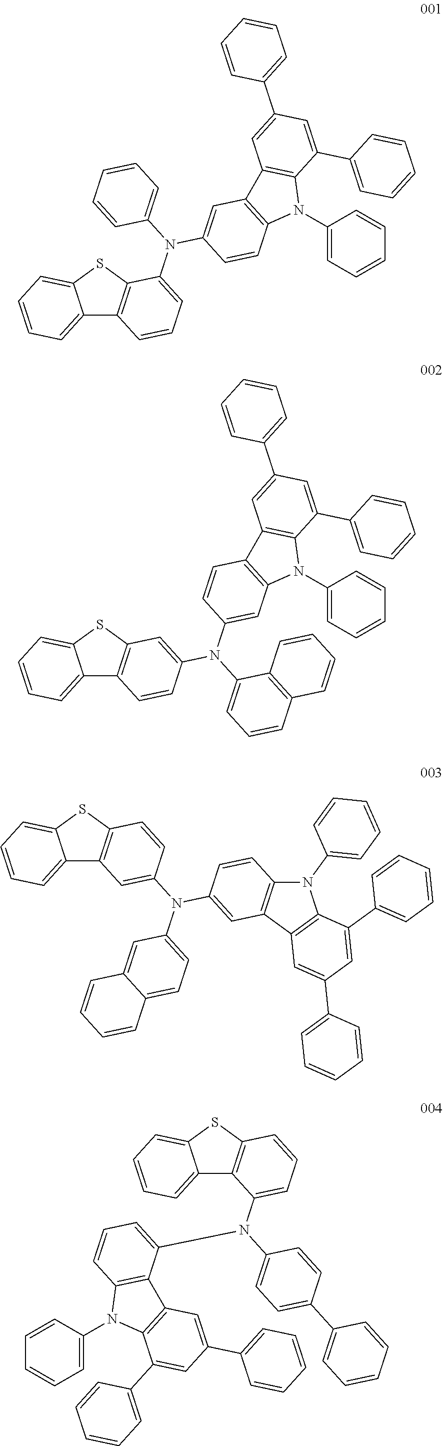 [SMILES: c1ccc(-c2cc(-c3ccccc3)c3c(c2)c2cc(N(c4ccccc4)c4cccc5c4sc4ccccc45)ccc2n3-c2ccccc2)cc1 ; c1ccc(-c2cc(-c3ccccc3)c3c(c2)c2ccc(N(c4ccc5c(c4)sc4ccccc45)c4cccc5ccccc45)cc2n3-c2ccccc2)cc1 ; c1ccc(-c2cc(-c3ccccc3)c3c(c2)c2cc(N(c4ccc5ccccc5c4)c4ccc5sc6ccccc6c5c4)ccc2n3-c2ccccc2)cc1 ; c1ccc(-c2ccc(N(c3cccc4sc5ccccc5c34)c3cccc4c3c3cc(-c5ccccc5)cc(-c5ccccc5)c3n4-c3ccccc3)cc2)cc1]

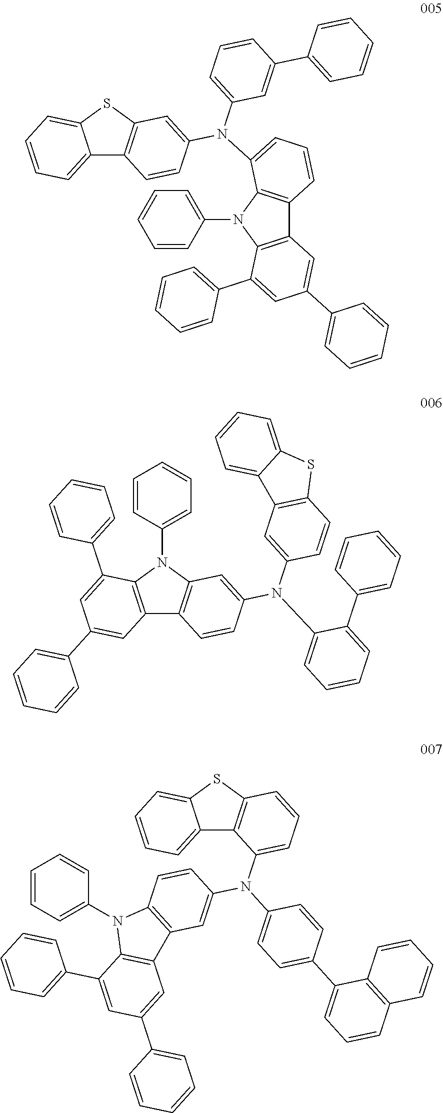 [SMILES: c1ccc(-c2cccc(N(c3ccc4c(c3)sc3ccccc34)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccc5sc6ccccc6c5c4)c4ccccc4-c4ccccc4)cc2n3-c2ccccc2)cc1 ; c1ccc(-c2cc(-c3ccccc3)c3c(c2)c2cc(N(c4ccc(-c5cccc6ccccc56)cc4)c4cccc5sc6ccccc6c45)ccc2n3-c2ccccc2)cc1]

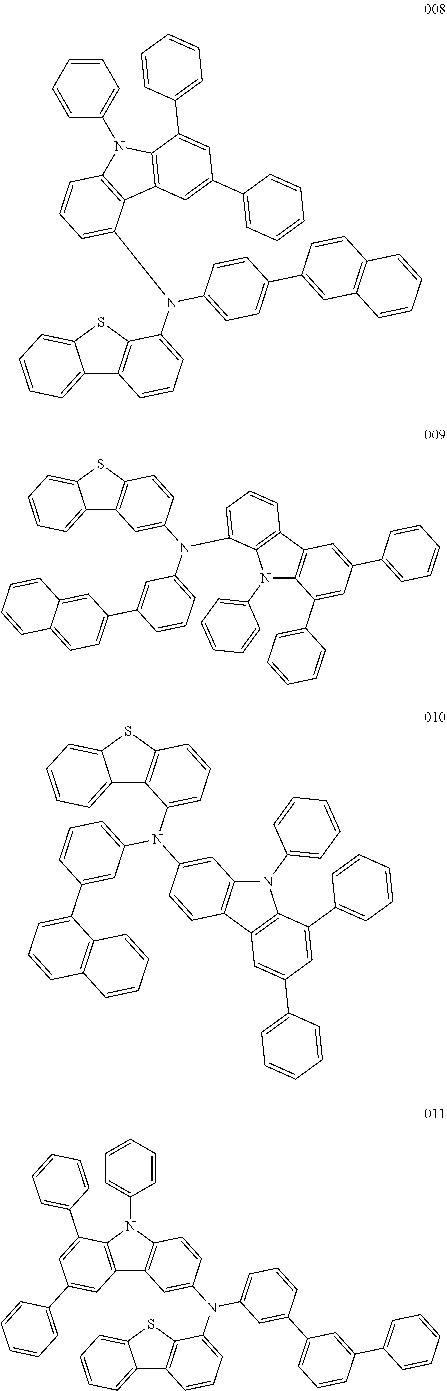

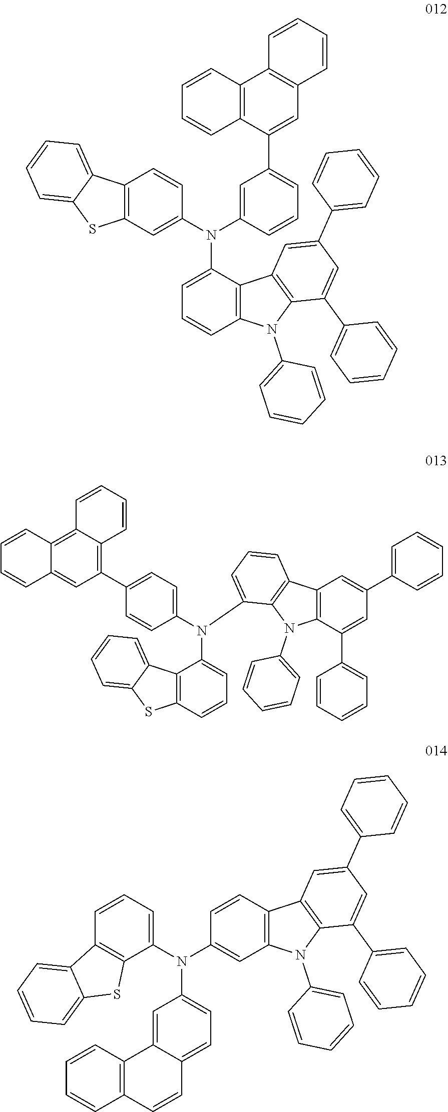 [SMILES: c1ccc(-c2cc(-c3ccccc3)c3c(c2)c2c(N(c4cccc(-c5cc6ccccc6c6ccccc56)c4)c4ccc5c(c4)sc4ccccc45)cccc2n3-c2ccccc2)cc1 ; c1ccc(-c2cc(-c3ccccc3)c3c(c2)c2cccc(N(c4ccc(-c5cc6ccccc6c6ccccc56)cc4)c4cccc5sc6ccccc6c45)c2n3-c2ccccc2)cc1 ; c1ccc(-c2cc(-c3ccccc3)c3c(c2)c2ccc(N(c4ccc5ccc6ccccc6c5c4)c4cccc5c4sc4ccccc45)cc2n3-c2ccccc2)cc1]

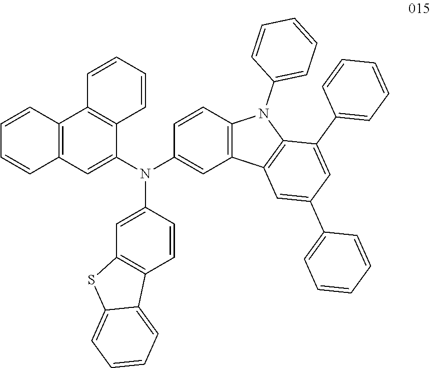 [SMILES: c1ccc(-c2cc(-c3ccccc3)c3c(c2)c2cc(N(c4ccc5c(c4)sc4ccccc45)c4cc5ccccc5c5ccccc45)ccc2n3-c2ccccc2)cc1]

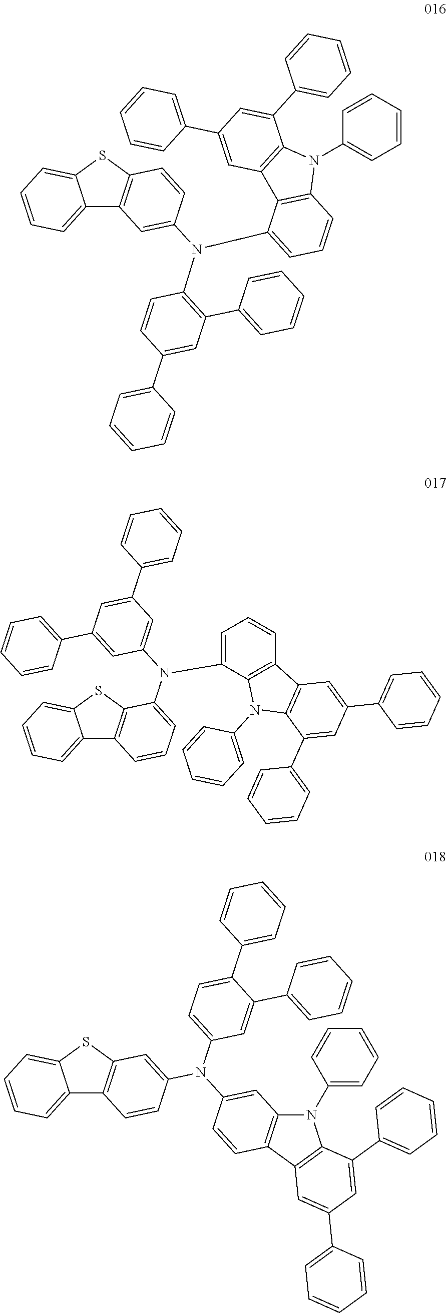 [SMILES: c1ccc(-c2ccc(N(c3ccc4sc5ccccc5c4c3)c3cccc4c3c3cc(-c5ccccc5)cc(-c5ccccc5)c3n4-c3ccccc3)c(-c3ccccc3)c2)cc1 ; c1ccc(-c2cc(-c3ccccc3)cc(N(c3cccc4c3sc3ccccc34)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccc(-c5ccccc5)c(-c5ccccc5)c4)c4ccc5c(c4)sc4ccccc45)cc2n3-c2ccccc2)cc1]

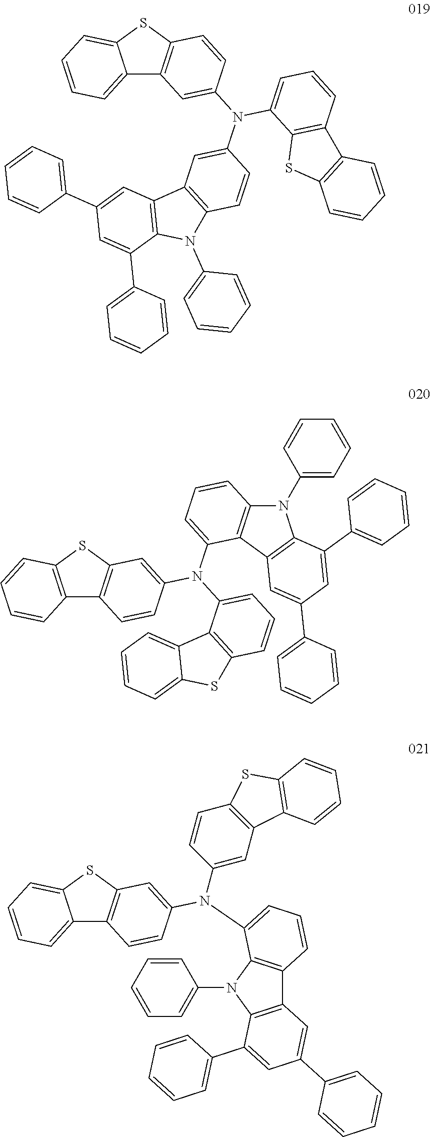 [SMILES: c1ccc(-c2cc(-c3ccccc3)c3c(c2)c2cc(N(c4ccc5sc6ccccc6c5c4)c4cccc5c4sc4ccccc45)ccc2n3-c2ccccc2)cc1 ; c1ccc(-c2cc(-c3ccccc3)c3c(c2)c2c(N(c4ccc5c(c4)sc4ccccc45)c4cccc5sc6ccccc6c45)cccc2n3-c2ccccc2)cc1 ; c1ccc(-c2cc(-c3ccccc3)c3c(c2)c2cccc(N(c4ccc5c(c4)sc4ccccc45)c4ccc5sc6ccccc6c5c4)c2n3-c2ccccc2)cc1]

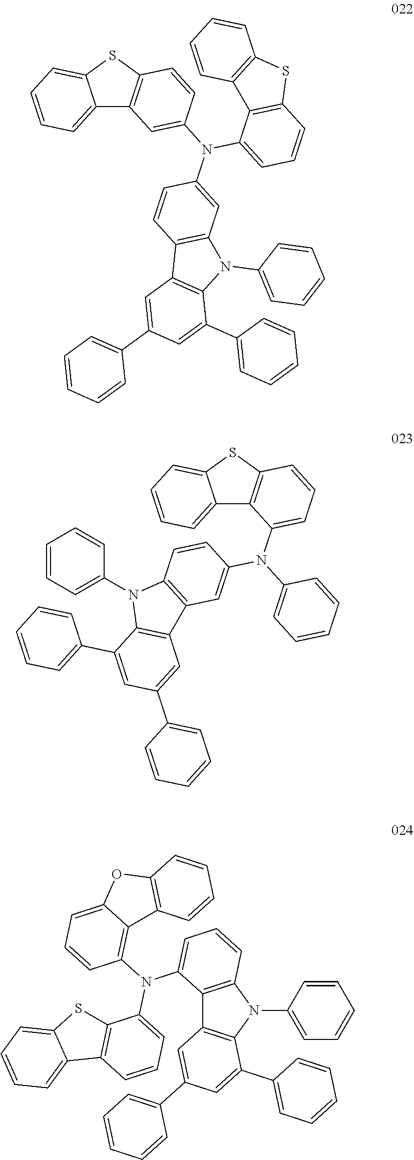 [SMILES: c1ccc(-c2cc(-c3ccccc3)c3c(c2)c2ccc(N(c4ccc5sc6ccccc6c5c4)c4cccc5sc6ccccc6c45)cc2n3-c2ccccc2)cc1 ; c1ccc(-c2cc(-c3ccccc3)c3c(c2)c2cc(N(c4ccccc4)c4cccc5sc6ccccc6c45)ccc2n3-c2ccccc2)cc1 ; c1ccc(-c2cc(-c3ccccc3)c3c(c2)c2c(N(c4cccc5c4sc4ccccc45)c4cccc5oc6ccccc6c45)cccc2n3-c2ccccc2)cc1]

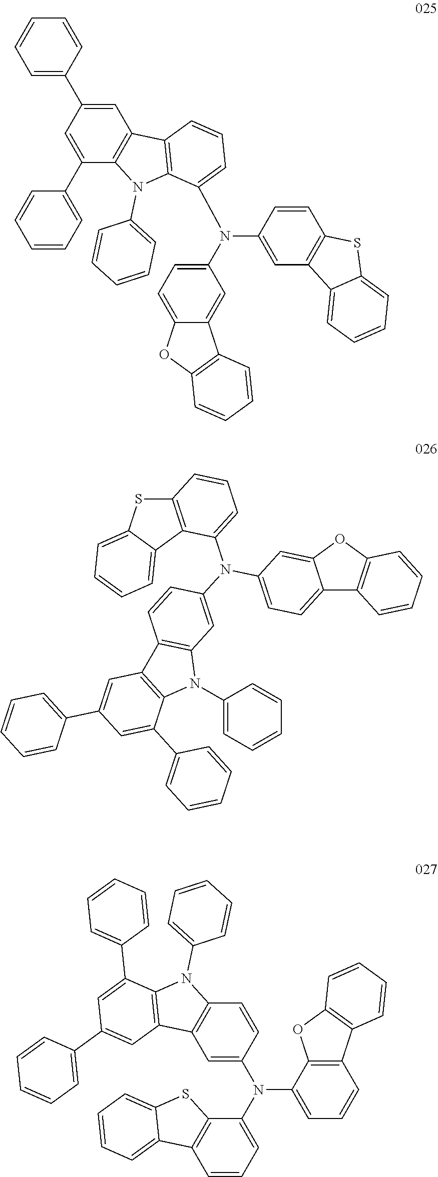 [SMILES: c1ccc(-c2cc(-c3ccccc3)c3c(c2)c2cccc(N(c4ccc5oc6ccccc6c5c4)c4ccc5sc6ccccc6c5c4)c2n3-c2ccccc2)cc1 ; c1ccc(-c2cc(-c3ccccc3)c3c(c2)c2ccc(N(c4ccc5c(c4)oc4ccccc45)c4cccc5sc6ccccc6c45)cc2n3-c2ccccc2)cc1 ; c1ccc(-c2cc(-c3ccccc3)c3c(c2)c2cc(N(c4cccc5c4oc4ccccc45)c4cccc5c4sc4ccccc45)ccc2n3-c2ccccc2)cc1]

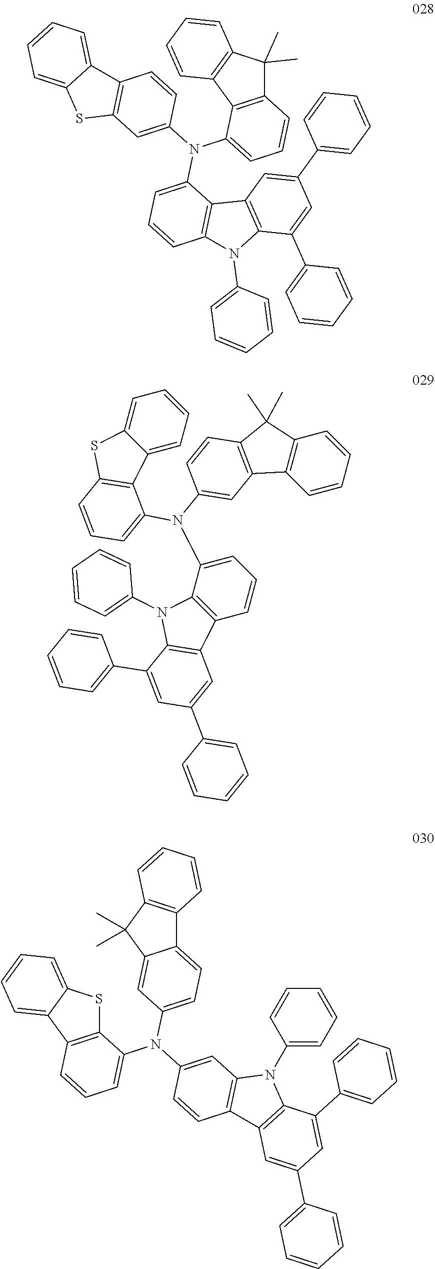

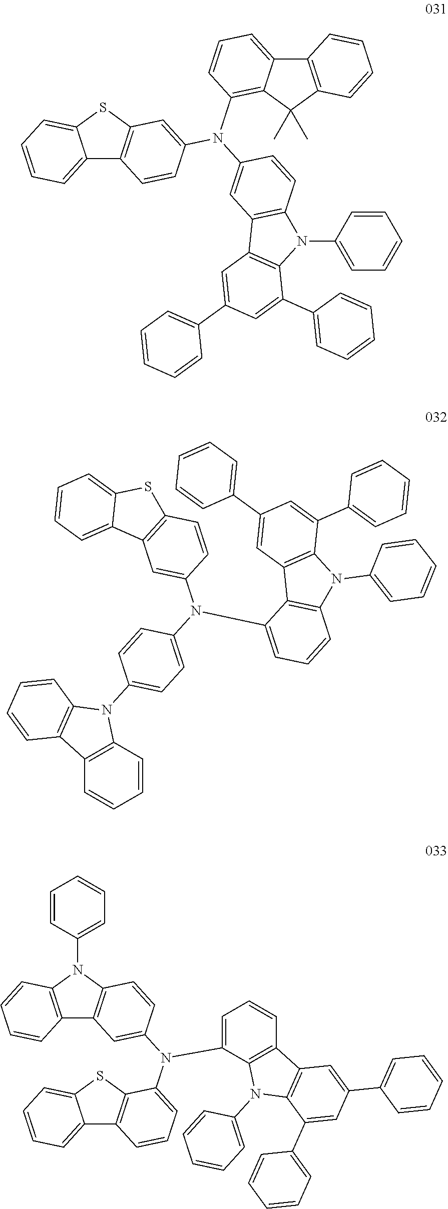 [SMILES: CC1(C)c2ccccc2-c2cccc(N(c3ccc4c(c3)sc3ccccc34)c3ccc4c(c3)c3cc(-c5ccccc5)cc(-c5ccccc5)c3n4-c3ccccc3)c21 ; c1ccc(-c2cc(-c3ccccc3)c3c(c2)c2c(N(c4ccc(-n5c6ccccc6c6ccccc65)cc4)c4ccc5sc6ccccc6c5c4)cccc2n3-c2ccccc2)cc1 ; c1ccc(-c2cc(-c3ccccc3)c3c(c2)c2cccc(N(c4ccc5c(c4)c4ccccc4n5-c4ccccc4)c4cccc5c4sc4ccccc45)c2n3-c2ccccc2)cc1]

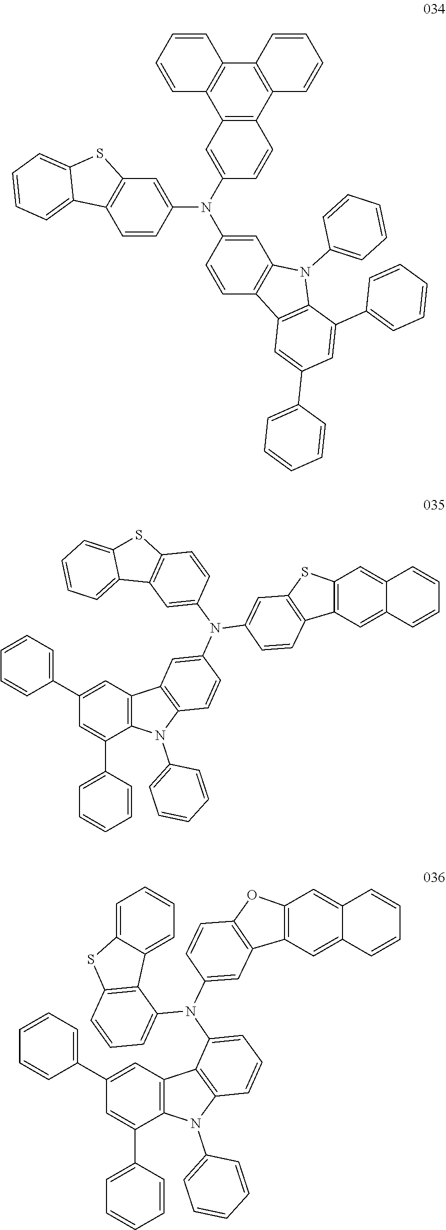 [SMILES: c1ccc(-c2cc(-c3ccccc3)c3c(c2)c2ccc(N(c4ccc5c(c4)sc4ccccc45)c4ccc5c6ccccc6c6ccccc6c5c4)cc2n3-c2ccccc2)cc1 ; c1ccc(-c2cc(-c3ccccc3)c3c(c2)c2cc(N(c4ccc5c(c4)sc4cc6ccccc6cc45)c4ccc5sc6ccccc6c5c4)ccc2n3-c2ccccc2)cc1 ; c1ccc(-c2cc(-c3ccccc3)c3c(c2)c2c(N(c4ccc5oc6cc7ccccc7cc6c5c4)c4cccc5sc6ccccc6c45)cccc2n3-c2ccccc2)cc1]

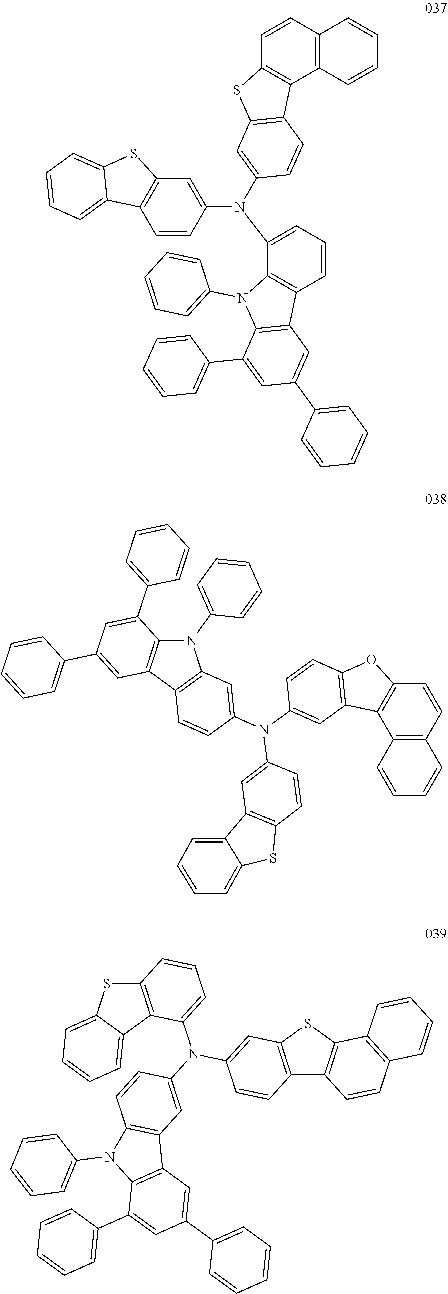 [SMILES: c1ccc(-c2cc(-c3ccccc3)c3c(c2)c2cccc(N(c4ccc5c(c4)sc4ccccc45)c4ccc5c(c4)sc4ccc6ccccc6c45)c2n3-c2ccccc2)cc1 ; c1ccc(-c2cc(-c3ccccc3)c3c(c2)c2ccc(N(c4ccc5sc6ccccc6c5c4)c4ccc5oc6ccc7ccccc7c6c5c4)cc2n3-c2ccccc2)cc1 ; c1ccc(-c2cc(-c3ccccc3)c3c(c2)c2cc(N(c4ccc5c(c4)sc4c6ccccc6ccc54)c4cccc5sc6ccccc6c45)ccc2n3-c2ccccc2)cc1]

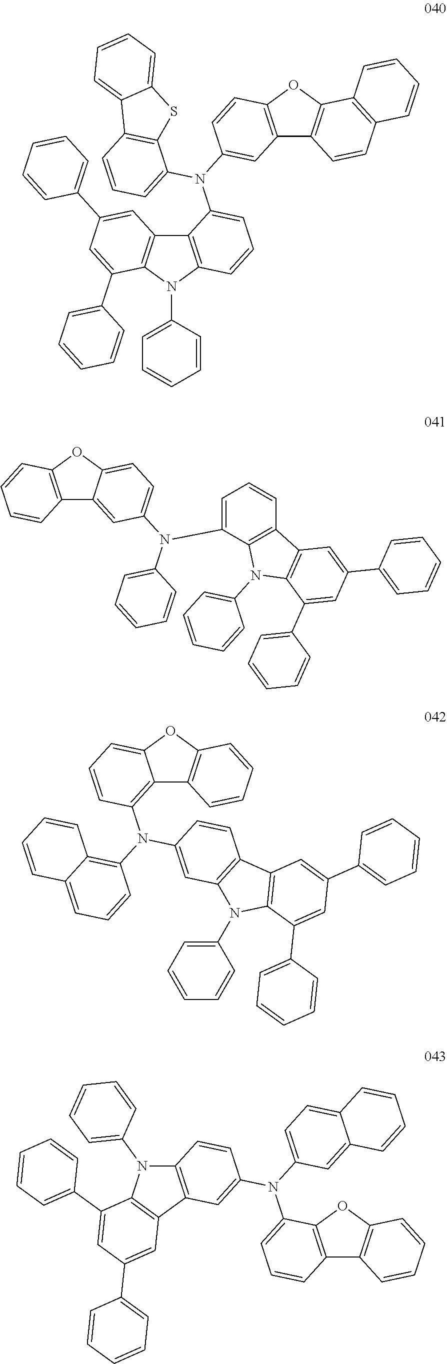 [SMILES: c1ccc(-c2cc(-c3ccccc3)c3c(c2)c2c(N(c4ccc5oc6c7ccccc7ccc6c5c4)c4cccc5c4sc4ccccc45)cccc2n3-c2ccccc2)cc1 ; c1ccc(-c2cc(-c3ccccc3)c3c(c2)c2cccc(N(c4ccccc4)c4ccc5oc6ccccc6c5c4)c2n3-c2ccccc2)cc1 ; c1ccc(-c2cc(-c3ccccc3)c3c(c2)c2ccc(N(c4cccc5ccccc45)c4cccc5oc6ccccc6c45)cc2n3-c2ccccc2)cc1 ; c1ccc(-c2cc(-c3ccccc3)c3c(c2)c2cc(N(c4ccc5ccccc5c4)c4cccc5c4oc4ccccc45)ccc2n3-c2ccccc2)cc1]

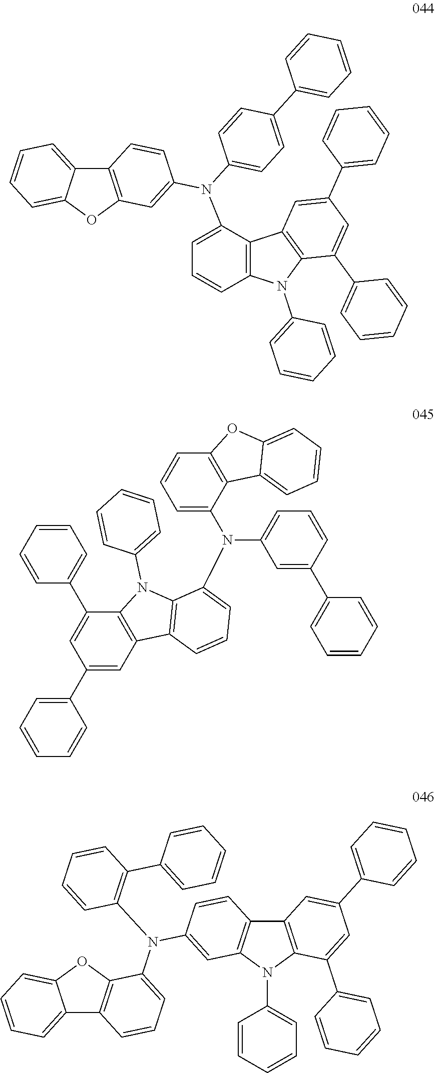 [SMILES: c1ccc(-c2ccc(N(c3ccc4c(c3)oc3ccccc34)c3cccc4c3c3cc(-c5ccccc5)cc(-c5ccccc5)c3n4-c3ccccc3)cc2)cc1 ; c1ccc(-c2cccc(N(c3cccc4oc5ccccc5c34)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccccc4-c4ccccc4)c4cccc5c4oc4ccccc45)cc2n3-c2ccccc2)cc1]

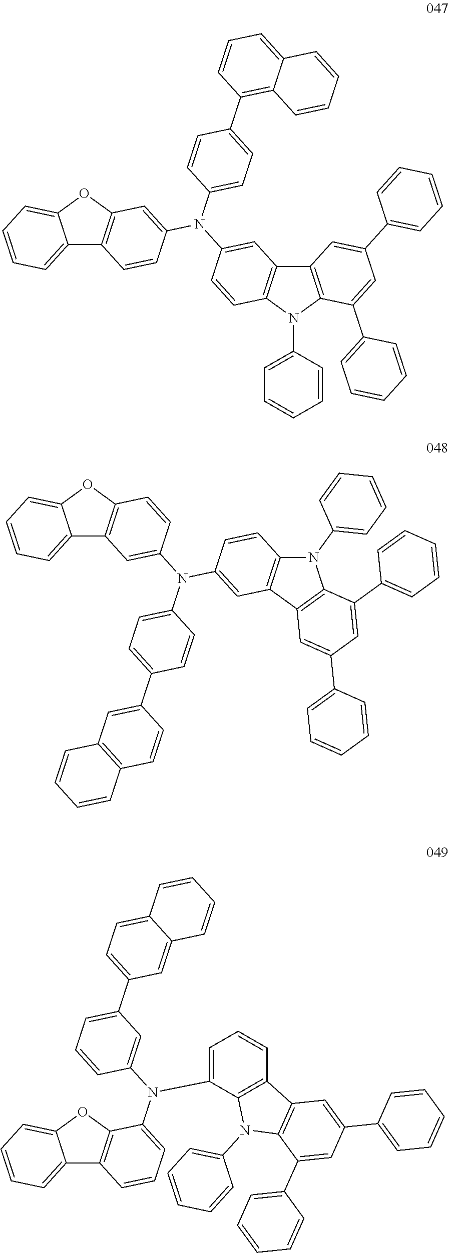 [SMILES: c1ccc(-c2cc(-c3ccccc3)c3c(c2)c2cc(N(c4ccc(-c5cccc6ccccc56)cc4)c4ccc5c(c4)oc4ccccc45)ccc2n3-c2ccccc2)cc1 ; c1ccc(-c2cc(-c3ccccc3)c3c(c2)c2cc(N(c4ccc(-c5ccc6ccccc6c5)cc4)c4ccc5oc6ccccc6c5c4)ccc2n3-c2ccccc2)cc1 ; c1ccc(-c2cc(-c3ccccc3)c3c(c2)c2cccc(N(c4cccc(-c5ccc6ccccc6c5)c4)c4cccc5c4oc4ccccc45)c2n3-c2ccccc2)cc1]

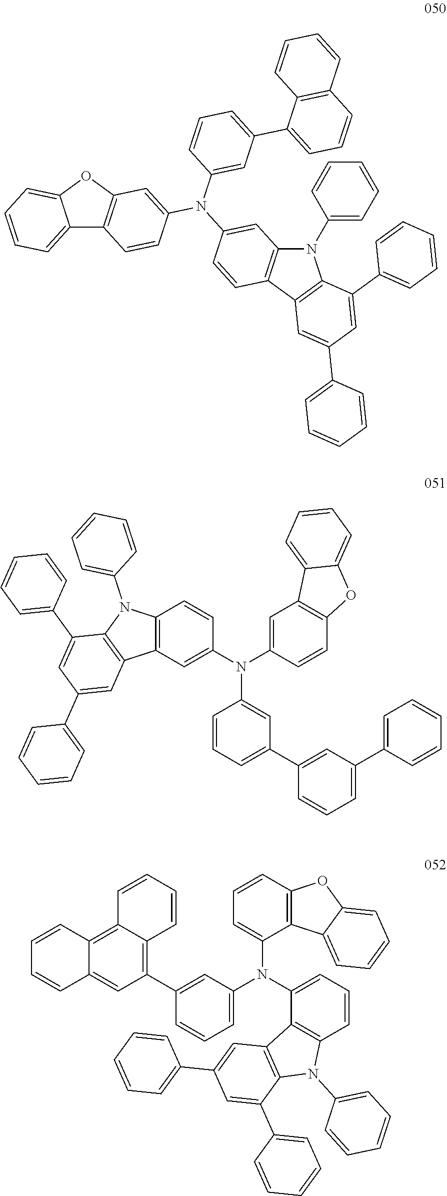 [SMILES: c1ccc(-c2cc(-c3ccccc3)c3c(c2)c2ccc(N(c4cccc(-c5cccc6ccccc56)c4)c4ccc5c(c4)oc4ccccc45)cc2n3-c2ccccc2)cc1 ; c1ccc(-c2cccc(-c3cccc(N(c4ccc5oc6ccccc6c5c4)c4ccc5c(c4)c4cc(-c6ccccc6)cc(-c6ccccc6)c4n5-c4ccccc4)c3)c2)cc1 ; c1ccc(-c2cc(-c3ccccc3)c3c(c2)c2c(N(c4cccc(-c5cc6ccccc6c6ccccc56)c4)c4cccc5oc6ccccc6c45)cccc2n3-c2ccccc2)cc1]

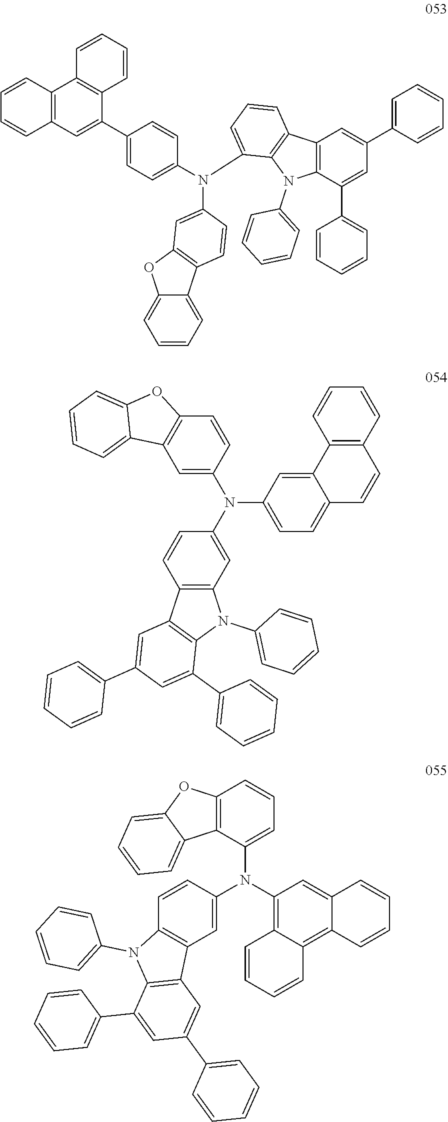

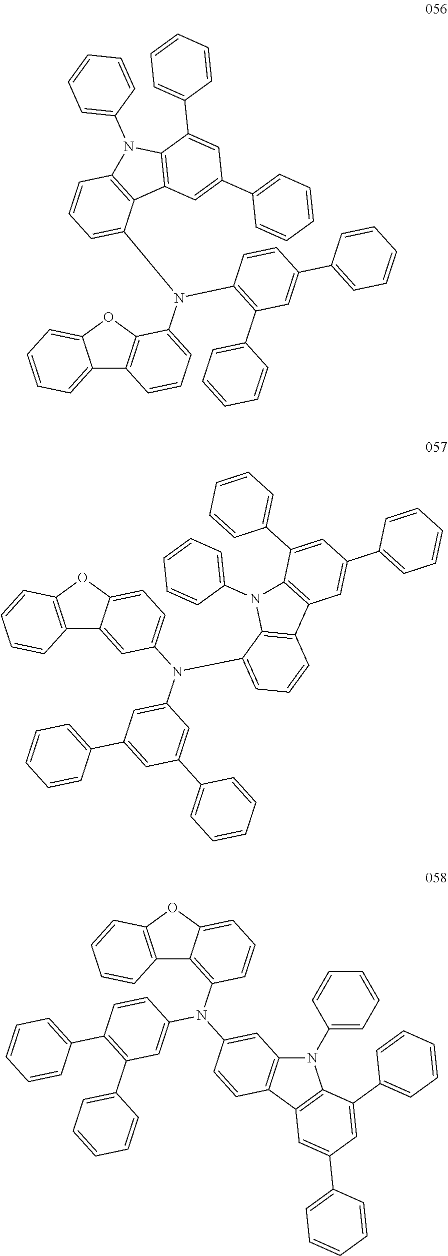 [SMILES: c1ccc(-c2ccc(N(c3cccc4c3oc3ccccc34)c3cccc4c3c3cc(-c5ccccc5)cc(-c5ccccc5)c3n4-c3ccccc3)c(-c3ccccc3)c2)cc1 ; c1ccc(-c2cc(-c3ccccc3)cc(N(c3ccc4oc5ccccc5c4c3)c3cccc4c5cc(-c6ccccc6)cc(-c6ccccc6)c5n(-c5ccccc5)c34)c2)cc1 ; c1ccc(-c2cc(-c3ccccc3)c3c(c2)c2ccc(N(c4ccc(-c5ccccc5)c(-c5ccccc5)c4)c4cccc5oc6ccccc6c45)cc2n3-c2ccccc2)cc1]

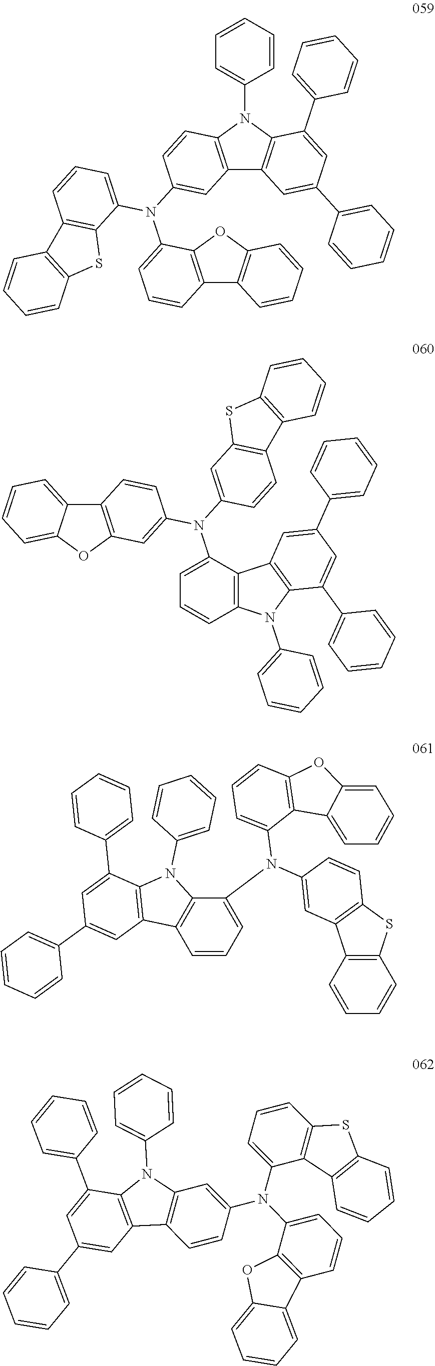 [SMILES: c1ccc(-c2cc(-c3ccccc3)c3c(c2)c2cc(N(c4cccc5c4oc4ccccc45)c4cccc5c4sc4ccccc45)ccc2n3-c2ccccc2)cc1 ; c1ccc(-c2cc(-c3ccccc3)c3c(c2)c2c(N(c4ccc5c(c4)oc4ccccc45)c4ccc5c(c4)sc4ccccc45)cccc2n3-c2ccccc2)cc1 ; c1ccc(-c2cc(-c3ccccc3)c3c(c2)c2cccc(N(c4ccc5sc6ccccc6c5c4)c4cccc5oc6ccccc6c45)c2n3-c2ccccc2)cc1 ; c1ccc(-c2cc(-c3ccccc3)c3c(c2)c2ccc(N(c4cccc5c4oc4ccccc45)c4cccc5sc6ccccc6c45)cc2n3-c2ccccc2)cc1]

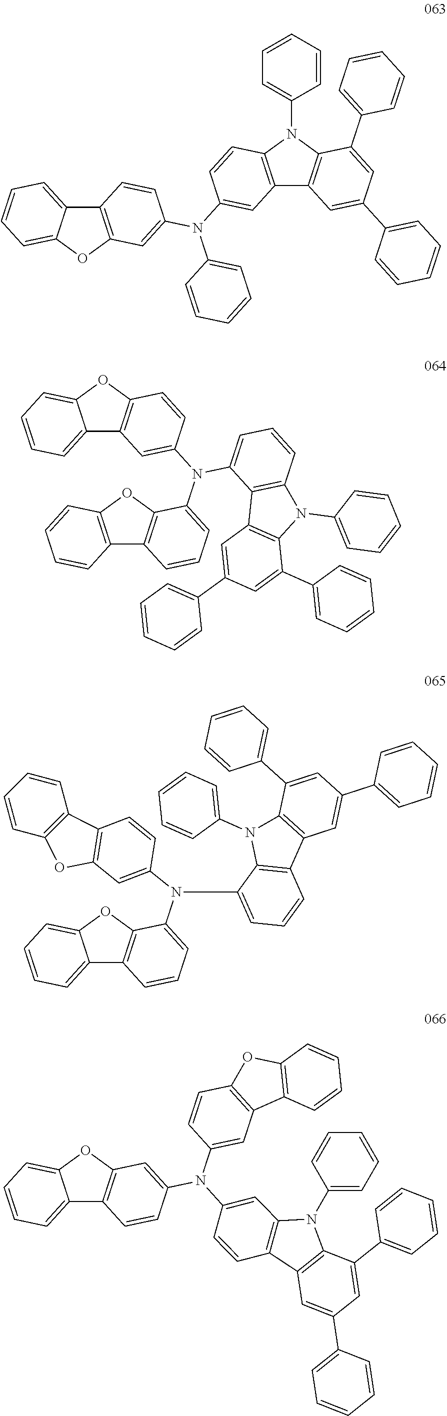 [SMILES: c1ccc(-c2cc(-c3ccccc3)c3c(c2)c2cc(N(c4ccccc4)c4ccc5c(c4)oc4ccccc45)ccc2n3-c2ccccc2)cc1 ; c1ccc(-c2cc(-c3ccccc3)c3c(c2)c2c(N(c4ccc5oc6ccccc6c5c4)c4cccc5c4oc4ccccc45)cccc2n3-c2ccccc2)cc1 ; c1ccc(-c2cc(-c3ccccc3)c3c(c2)c2cccc(N(c4ccc5c(c4)oc4ccccc45)c4cccc5c4oc4ccccc45)c2n3-c2ccccc2)cc1 ; c1ccc(-c2cc(-c3ccccc3)c3c(c2)c2ccc(N(c4ccc5c(c4)oc4ccccc45)c4ccc5oc6ccccc6c5c4)cc2n3-c2ccccc2)cc1]

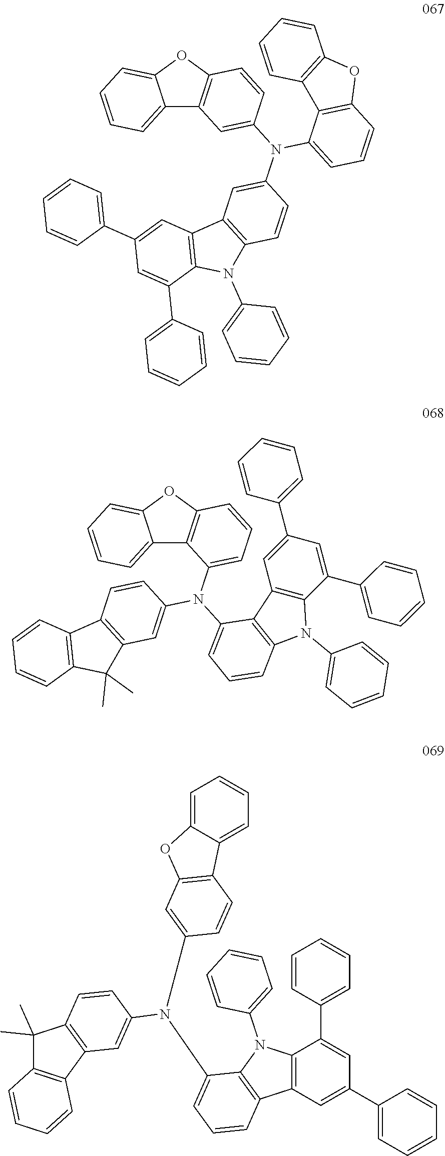 [SMILES: c1ccc(-c2cc(-c3ccccc3)c3c(c2)c2cc(N(c4ccc5oc6ccccc6c5c4)c4cccc5oc6ccccc6c45)ccc2n3-c2ccccc2)cc1 ; CC1(C)c2ccccc2-c2ccc(N(c3cccc4oc5ccccc5c34)c3cccc4c3c3cc(-c5ccccc5)cc(-c5ccccc5)c3n4-c3ccccc3)cc21 ; CC1(C)c2ccccc2-c2cc(N(c3ccc4c(c3)oc3ccccc34)c3cccc4c5cc(-c6ccccc6)cc(-c6ccccc6)c5n(-c5ccccc5)c34)ccc21]

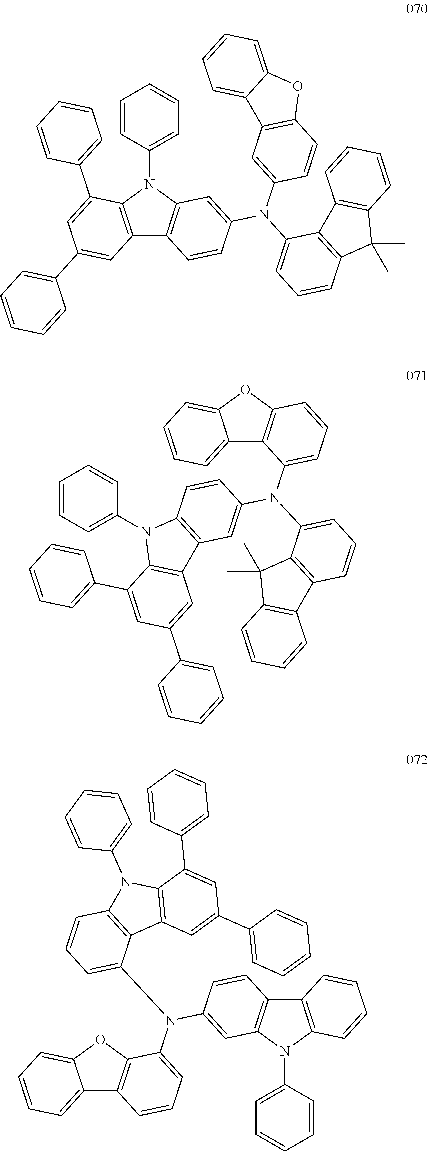 [SMILES: CC1(C)c2ccccc2-c2c(N(c3ccc4oc5ccccc5c4c3)c3ccc4c5cc(-c6ccccc6)cc(-c6ccccc6)c5n(-c5ccccc5)c4c3)cccc21 ; CC1(C)c2ccccc2-c2cccc(N(c3ccc4c(c3)c3cc(-c5ccccc5)cc(-c5ccccc5)c3n4-c3ccccc3)c3cccc4oc5ccccc5c34)c21 ; c1ccc(-c2cc(-c3ccccc3)c3c(c2)c2c(N(c4ccc5c6ccccc6n(-c6ccccc6)c5c4)c4cccc5c4oc4ccccc45)cccc2n3-c2ccccc2)cc1]

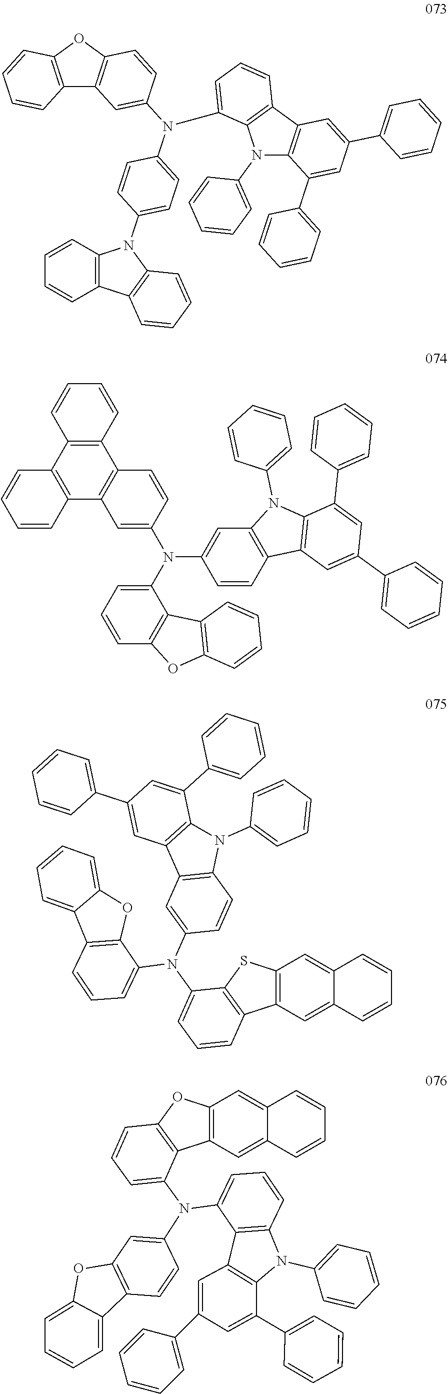

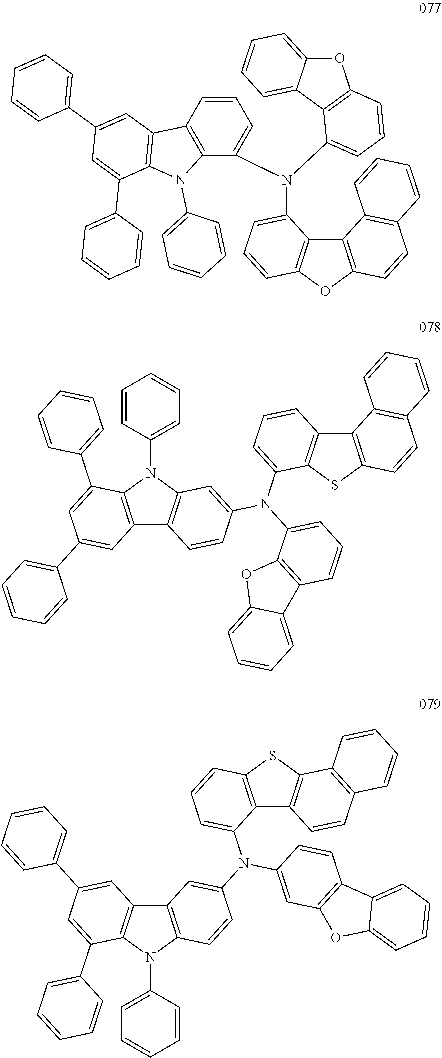 [SMILES: c1ccc(-c2cc(-c3ccccc3)c3c(c2)c2cccc(N(c4cccc5oc6ccccc6c45)c4cccc5oc6ccc7ccccc7c6c45)c2n3-c2ccccc2)cc1 ; c1ccc(-c2cc(-c3ccccc3)c3c(c2)c2ccc(N(c4cccc5c4oc4ccccc45)c4cccc5c4sc4ccc6ccccc6c45)cc2n3-c2ccccc2)cc1 ; c1ccc(-c2cc(-c3ccccc3)c3c(c2)c2cc(N(c4ccc5c(c4)oc4ccccc45)c4cccc5sc6c7ccccc7ccc6c45)ccc2n3-c2ccccc2)cc1]

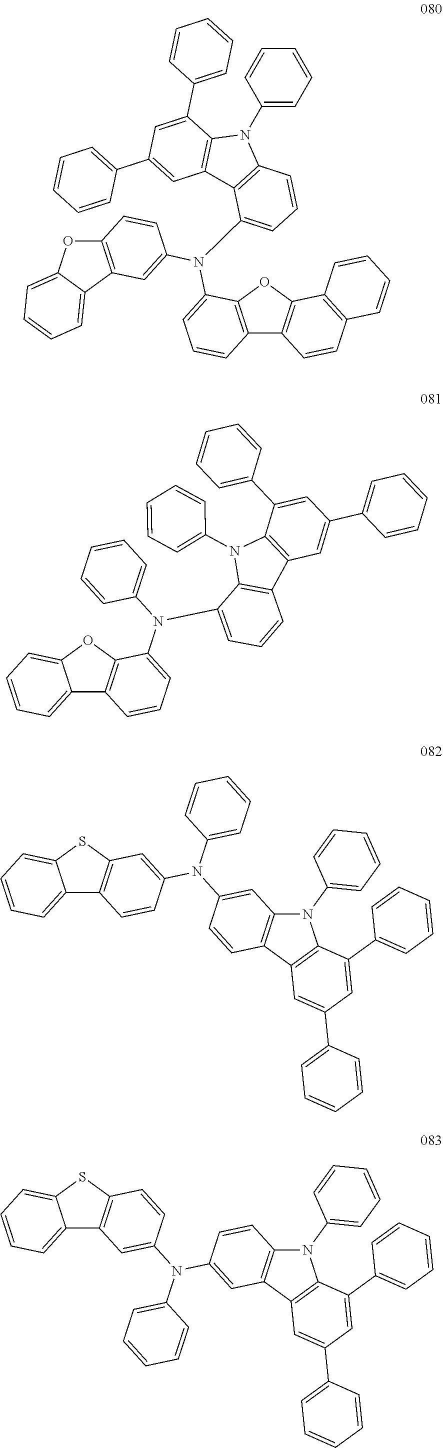 [SMILES: c1ccc(-c2cc(-c3ccccc3)c3c(c2)c2c(N(c4ccc5oc6ccccc6c5c4)c4cccc5c4oc4c6ccccc6ccc54)cccc2n3-c2ccccc2)cc1 ; c1ccc(-c2cc(-c3ccccc3)c3c(c2)c2cccc(N(c4ccccc4)c4cccc5c4oc4ccccc45)c2n3-c2ccccc2)cc1 ; c1ccc(-c2cc(-c3ccccc3)c3c(c2)c2ccc(N(c4ccccc4)c4ccc5c(c4)sc4ccccc45)cc2n3-c2ccccc2)cc1 ; c1ccc(-c2cc(-c3ccccc3)c3c(c2)c2cc(N(c4ccccc4)c4ccc5sc6ccccc6c5c4)ccc2n3-c2ccccc2)cc1]

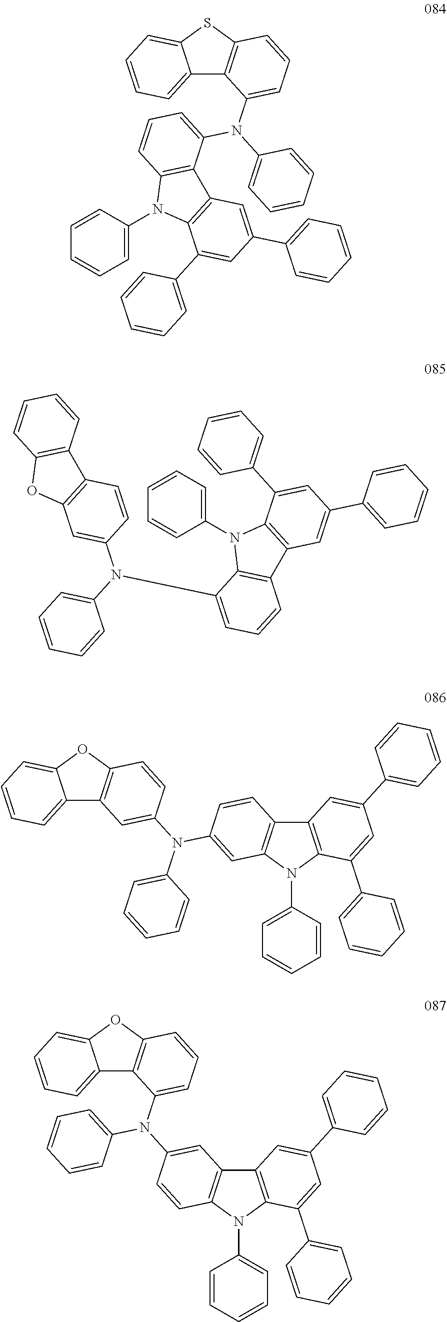 [SMILES: c1ccc(-c2cc(-c3ccccc3)c3c(c2)c2c(N(c4ccccc4)c4cccc5sc6ccccc6c45)cccc2n3-c2ccccc2)cc1 ; c1ccc(-c2cc(-c3ccccc3)c3c(c2)c2cccc(N(c4ccccc4)c4ccc5c(c4)oc4ccccc45)c2n3-c2ccccc2)cc1 ; c1ccc(-c2cc(-c3ccccc3)c3c(c2)c2ccc(N(c4ccccc4)c4ccc5oc6ccccc6c5c4)cc2n3-c2ccccc2)cc1 ; c1ccc(-c2cc(-c3ccccc3)c3c(c2)c2cc(N(c4ccccc4)c4cccc5oc6ccccc6c45)ccc2n3-c2ccccc2)cc1]

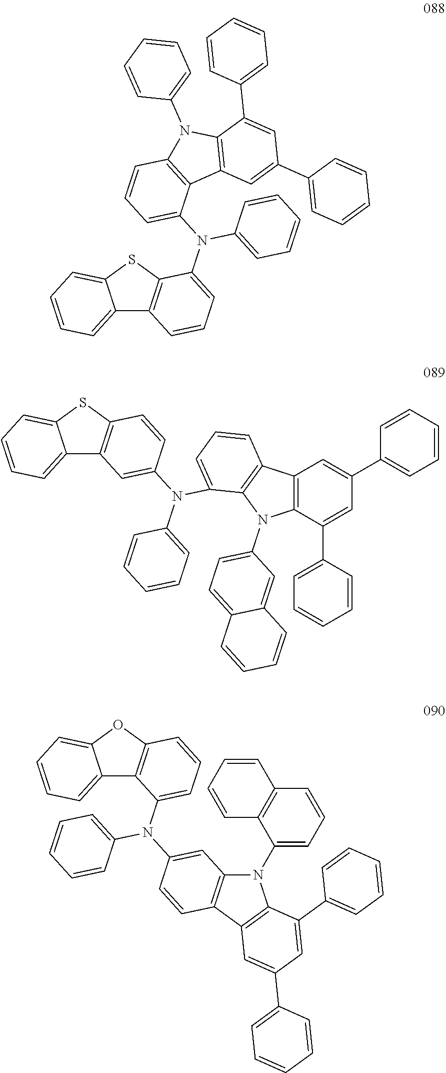 [SMILES: c1ccc(-c2cc(-c3ccccc3)c3c(c2)c2c(N(c4ccccc4)c4cccc5c4sc4ccccc45)cccc2n3-c2ccccc2)cc1 ; c1ccc(-c2cc(-c3ccccc3)c3c(c2)c2cccc(N(c4ccccc4)c4ccc5sc6ccccc6c5c4)c2n3-c2ccc3ccccc3c2)cc1 ; c1ccc(-c2cc(-c3ccccc3)c3c(c2)c2ccc(N(c4ccccc4)c4cccc5oc6ccccc6c45)cc2n3-c2cccc3ccccc23)cc1]

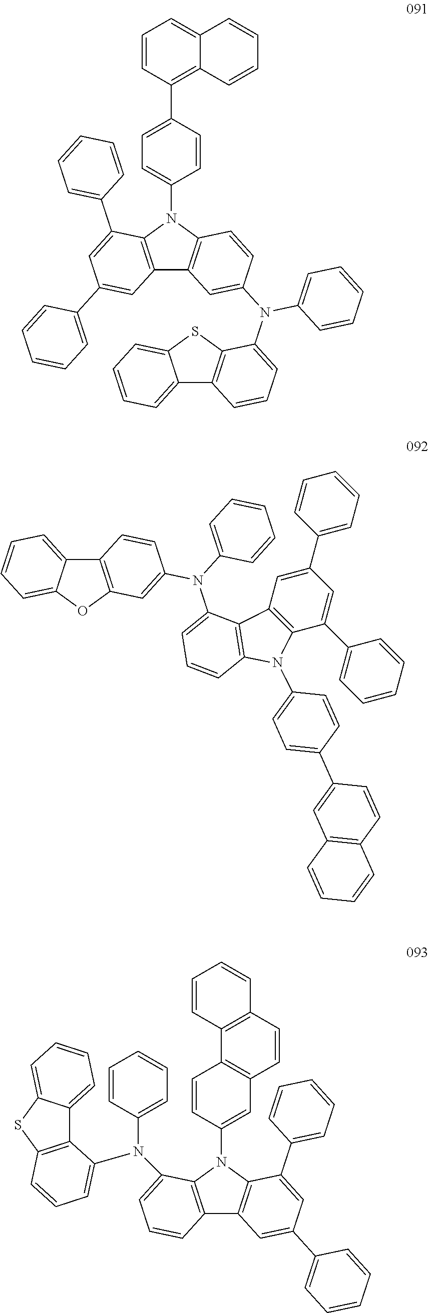 [SMILES: c1ccc(-c2cc(-c3ccccc3)c3c(c2)c2cc(N(c4ccccc4)c4cccc5c4sc4ccccc45)ccc2n3-c2ccc(-c3cccc4ccccc34)cc2)cc1 ; c1ccc(-c2cc(-c3ccccc3)c3c(c2)c2c(N(c4ccccc4)c4ccc5c(c4)oc4ccccc45)cccc2n3-c2ccc(-c3ccc4ccccc4c3)cc2)cc1 ; c1ccc(-c2cc(-c3ccccc3)c3c(c2)c2cccc(N(c4ccccc4)c4cccc5sc6ccccc6c45)c2n3-c2ccc3c(ccc4ccccc43)c2)cc1]

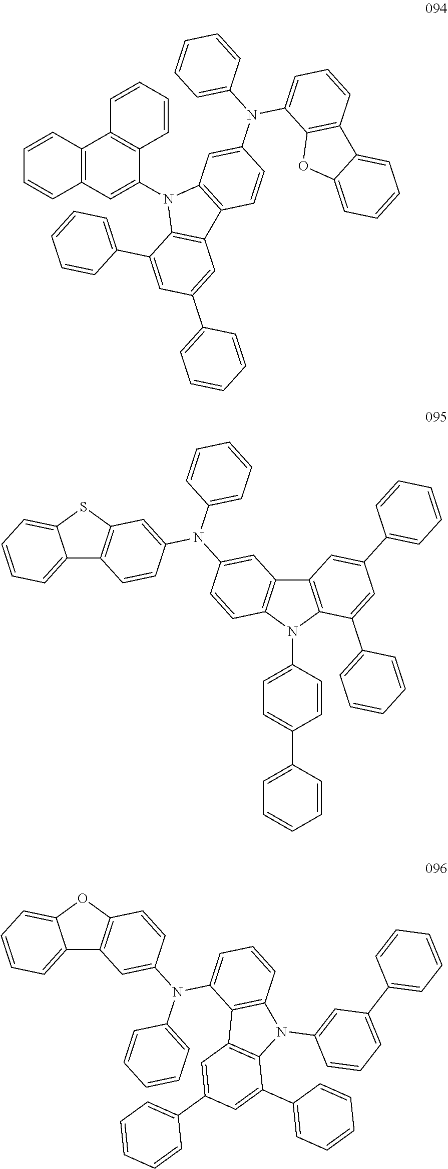 [SMILES: c1ccc(-c2cc(-c3ccccc3)c3c(c2)c2ccc(N(c4ccccc4)c4cccc5c4oc4ccccc45)cc2n3-c2cc3ccccc3c3ccccc23)cc1 ; c1ccc(-c2ccc(-n3c4ccc(N(c5ccccc5)c5ccc6c(c5)sc5ccccc56)cc4c4cc(-c5ccccc5)cc(-c5ccccc5)c43)cc2)cc1 ; c1ccc(-c2cccc(-n3c4cccc(N(c5ccccc5)c5ccc6oc7ccccc7c6c5)c4c4cc(-c5ccccc5)cc(-c5ccccc5)c43)c2)cc1]

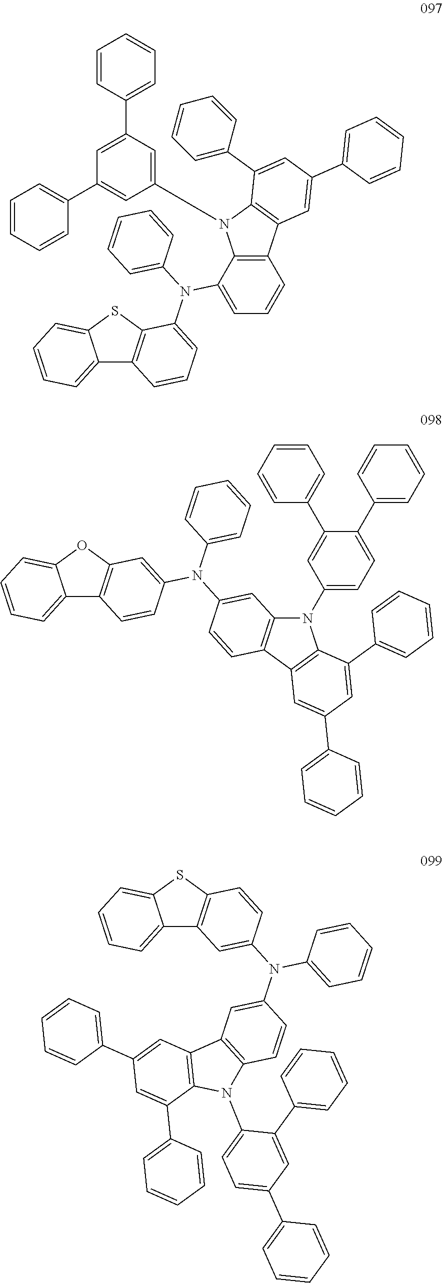 [SMILES: c1ccc(-c2cc(-c3ccccc3)cc(-n3c4c(-c5ccccc5)cc(-c5ccccc5)cc4c4cccc(N(c5ccccc5)c5cccc6c5sc5ccccc56)c43)c2)cc1 ; c1ccc(-c2cc(-c3ccccc3)c3c(c2)c2ccc(N(c4ccccc4)c4ccc5c(c4)oc4ccccc45)cc2n3-c2ccc(-c3ccccc3)c(-c3ccccc3)c2)cc1 ; c1ccc(-c2ccc(-n3c4ccc(N(c5ccccc5)c5ccc6sc7ccccc7c6c5)cc4c4cc(-c5ccccc5)cc(-c5ccccc5)c43)c(-c3ccccc3)c2)cc1]

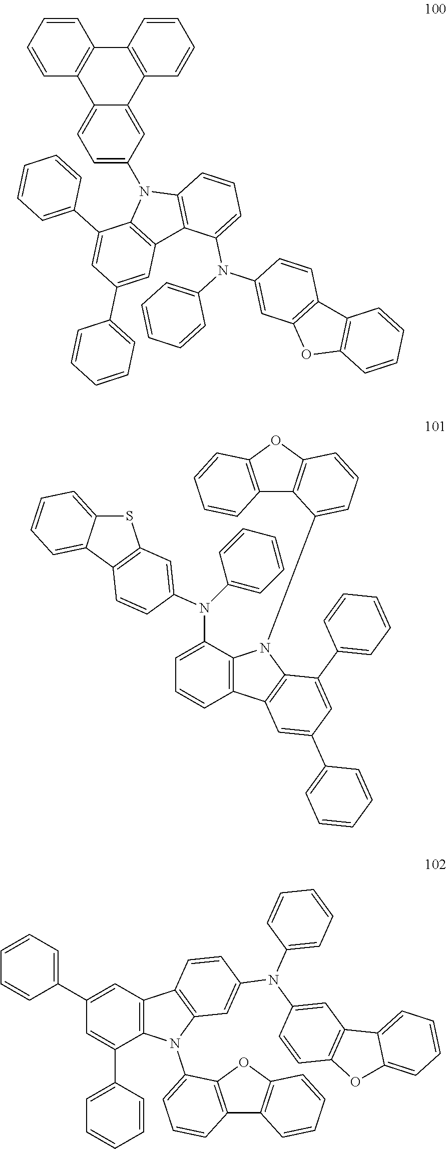 [SMILES: c1ccc(-c2cc(-c3ccccc3)c3c(c2)c2c(N(c4ccccc4)c4ccc5c(c4)oc4ccccc45)cccc2n3-c2ccc3c4ccccc4c4ccccc4c3c2)cc1 ; c1ccc(-c2cc(-c3ccccc3)c3c(c2)c2cccc(N(c4ccccc4)c4ccc5c(c4)sc4ccccc45)c2n3-c2cccc3oc4ccccc4c23)cc1 ; c1ccc(-c2cc(-c3ccccc3)c3c(c2)c2ccc(N(c4ccccc4)c4ccc5oc6ccccc6c5c4)cc2n3-c2cccc3c2oc2ccccc23)cc1]

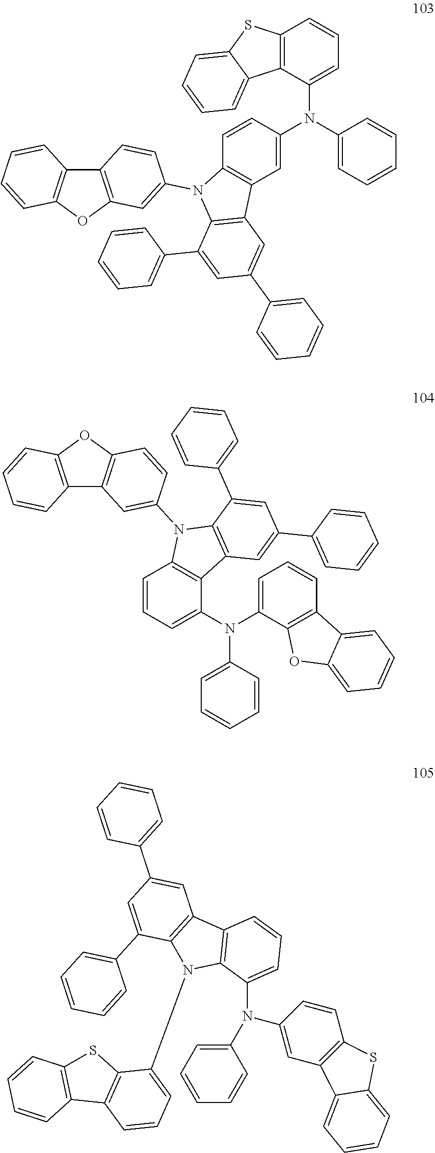 [SMILES: c1ccc(-c2cc(-c3ccccc3)c3c(c2)c2cc(N(c4ccccc4)c4cccc5sc6ccccc6c45)ccc2n3-c2ccc3c(c2)oc2ccccc23)cc1 ; c1ccc(-c2cc(-c3ccccc3)c3c(c2)c2c(N(c4ccccc4)c4cccc5c4oc4ccccc45)cccc2n3-c2ccc3oc4ccccc4c3c2)cc1 ; c1ccc(-c2cc(-c3ccccc3)c3c(c2)c2cccc(N(c4ccccc4)c4ccc5sc6ccccc6c5c4)c2n3-c2cccc3c2sc2ccccc23)cc1]

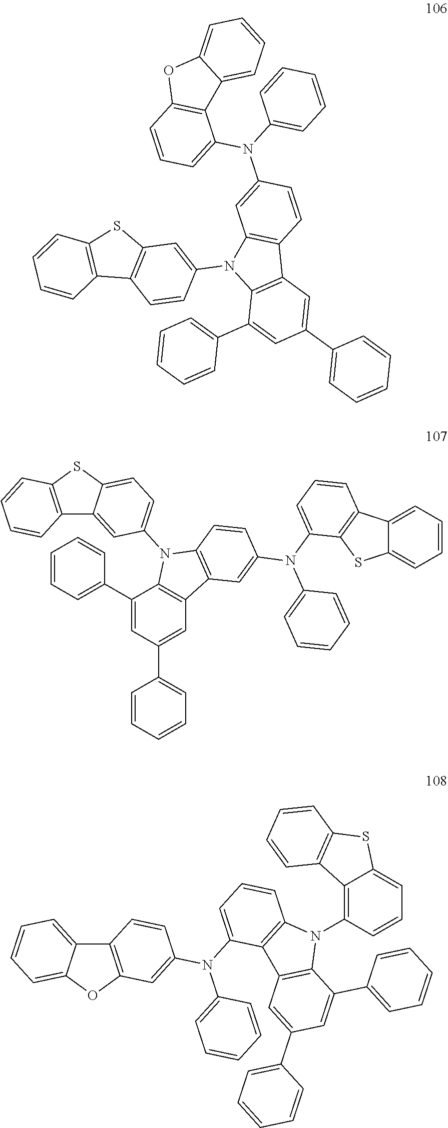 [SMILES: c1ccc(-c2cc(-c3ccccc3)c3c(c2)c2ccc(N(c4ccccc4)c4cccc5oc6ccccc6c45)cc2n3-c2ccc3c(c2)sc2ccccc23)cc1 ; c1ccc(-c2cc(-c3ccccc3)c3c(c2)c2cc(N(c4ccccc4)c4cccc5c4sc4ccccc45)ccc2n3-c2ccc3sc4ccccc4c3c2)cc1 ; c1ccc(-c2cc(-c3ccccc3)c3c(c2)c2c(N(c4ccccc4)c4ccc5c(c4)oc4ccccc45)cccc2n3-c2cccc3sc4ccccc4c23)cc1]

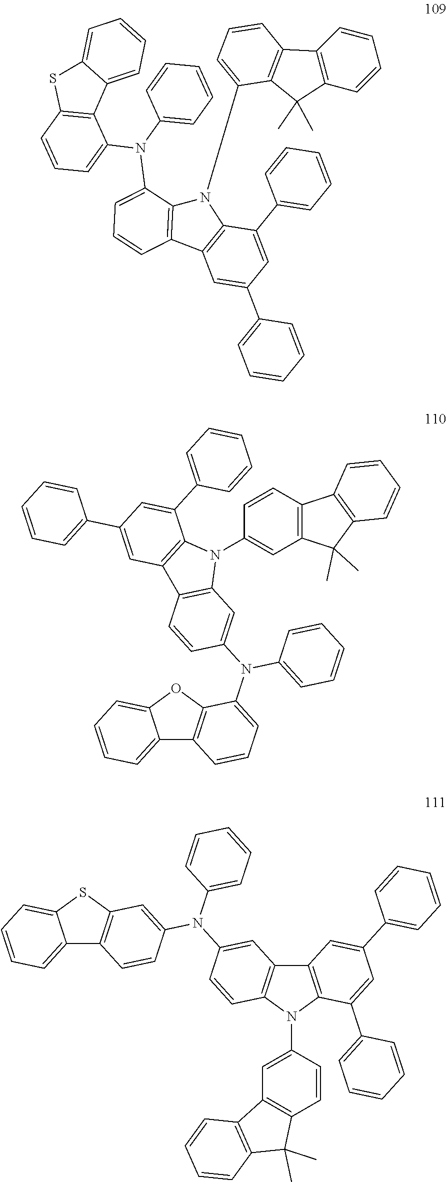 [SMILES: CC1(C)c2ccccc2-c2cccc(-n3c4c(-c5ccccc5)cc(-c5ccccc5)cc4c4cccc(N(c5ccccc5)c5cccc6sc7ccccc7c56)c43)c21 ; CC1(C)c2ccccc2-c2ccc(-n3c4cc(N(c5ccccc5)c5cccc6c5oc5ccccc56)ccc4c4cc(-c5ccccc5)cc(-c5ccccc5)c43)cc21 ; CC1(C)c2ccccc2-c2cc(-n3c4ccc(N(c5ccccc5)c5ccc6c(c5)sc5ccccc56)cc4c4cc(-c5ccccc5)cc(-c5ccccc5)c43)ccc21]

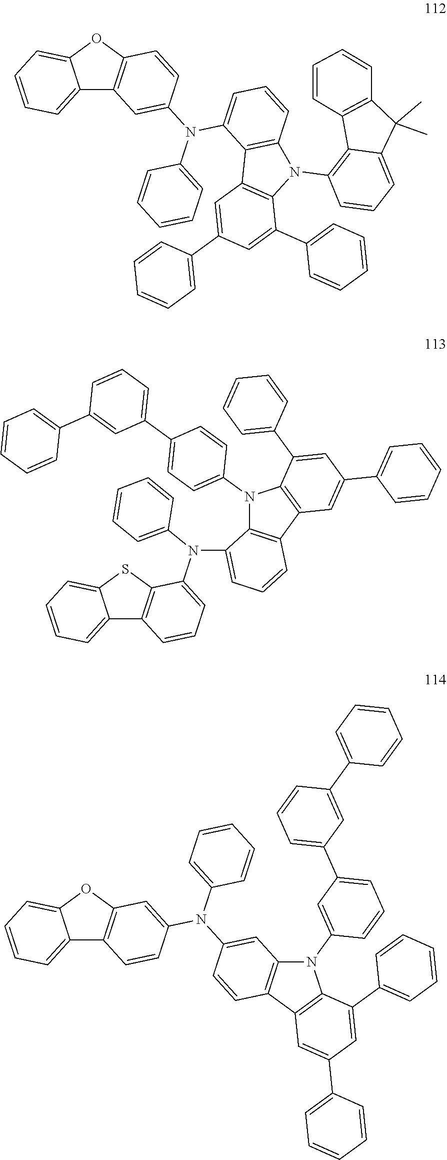 [SMILES: CC1(C)c2ccccc2-c2c(-n3c4cccc(N(c5ccccc5)c5ccc6oc7ccccc7c6c5)c4c4cc(-c5ccccc5)cc(-c5ccccc5)c43)cccc21 ; c1ccc(-c2cccc(-c3ccc(-n4c5c(-c6ccccc6)cc(-c6ccccc6)cc5c5cccc(N(c6ccccc6)c6cccc7c6sc6ccccc67)c54)cc3)c2)cc1 ; c1ccc(-c2cccc(-c3cccc(-n4c5cc(N(c6ccccc6)c6ccc7c(c6)oc6ccccc67)ccc5c5cc(-c6ccccc6)cc(-c6ccccc6)c54)c3)c2)cc1]

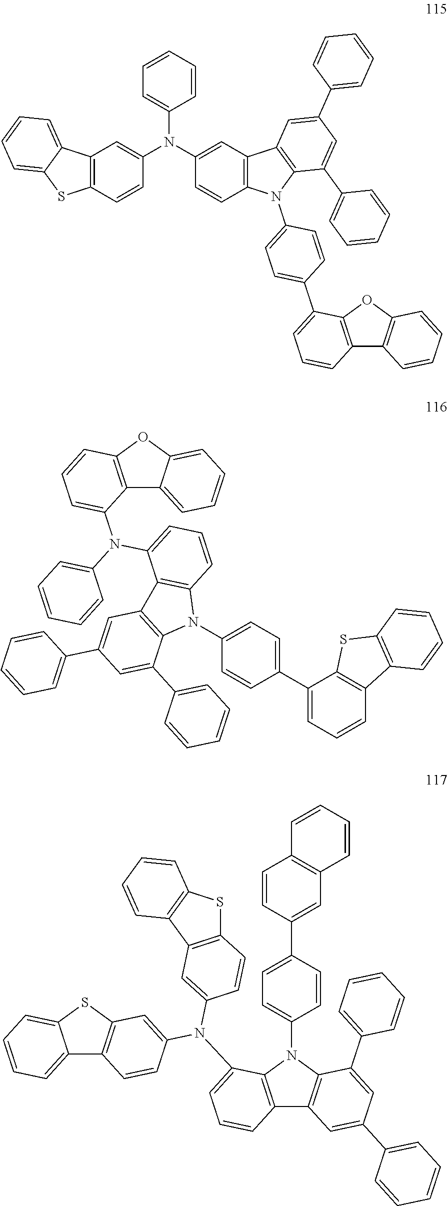 [SMILES: c1ccc(-c2cc(-c3ccccc3)c3c(c2)c2cc(N(c4ccccc4)c4ccc5sc6ccccc6c5c4)ccc2n3-c2ccc(-c3cccc4c3oc3ccccc34)cc2)cc1 ; c1ccc(-c2cc(-c3ccccc3)c3c(c2)c2c(N(c4ccccc4)c4cccc5oc6ccccc6c45)cccc2n3-c2ccc(-c3cccc4c3sc3ccccc34)cc2)cc1 ; c1ccc(-c2cc(-c3ccccc3)c3c(c2)c2cccc(N(c4ccc5c(c4)sc4ccccc45)c4ccc5sc6ccccc6c5c4)c2n3-c2ccc(-c3ccc4ccccc4c3)cc2)cc1]

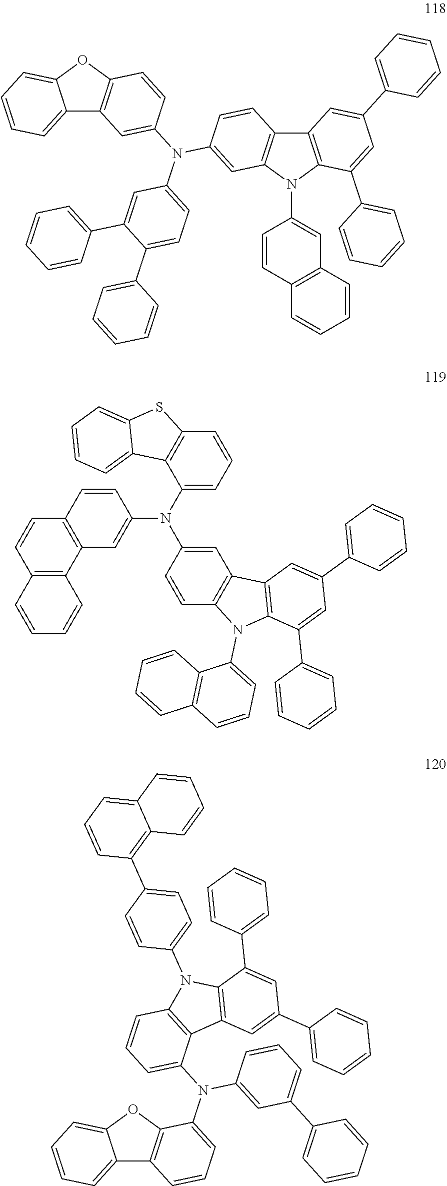 [SMILES: c1ccc(-c2cc(-c3ccccc3)c3c(c2)c2ccc(N(c4ccc(-c5ccccc5)c(-c5ccccc5)c4)c4ccc5oc6ccccc6c5c4)cc2n3-c2ccc3ccccc3c2)cc1 ; c1ccc(-c2cc(-c3ccccc3)c3c(c2)c2cc(N(c4ccc5ccc6ccccc6c5c4)c4cccc5sc6ccccc6c45)ccc2n3-c2cccc3ccccc23)cc1 ; c1ccc(-c2cccc(N(c3cccc4c3oc3ccccc34)c3cccc4c3c3cc(-c5ccccc5)cc(-c5ccccc5)c3n4-c3ccc(-c4cccc5ccccc45)cc3)c2)cc1]

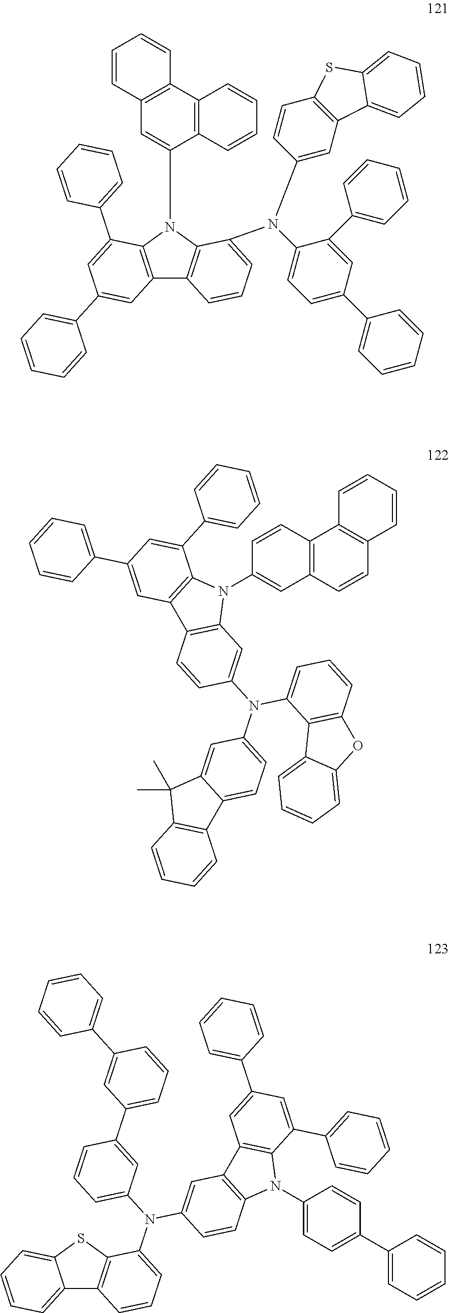 [SMILES: c1ccc(-c2ccc(N(c3ccc4sc5ccccc5c4c3)c3cccc4c5cc(-c6ccccc6)cc(-c6ccccc6)c5n(-c5cc6ccccc6c6ccccc56)c34)c(-c3ccccc3)c2)cc1 ; CC1(C)c2ccccc2-c2ccc(N(c3ccc4c5cc(-c6ccccc6)cc(-c6ccccc6)c5n(-c5ccc6c(ccc7ccccc76)c5)c4c3)c3cccc4oc5ccccc5c34)cc21 ; c1ccc(-c2ccc(-n3c4ccc(N(c5cccc(-c6cccc(-c7ccccc7)c6)c5)c5cccc6c5sc5ccccc56)cc4c4cc(-c5ccccc5)cc(-c5ccccc5)c43)cc2)cc1]

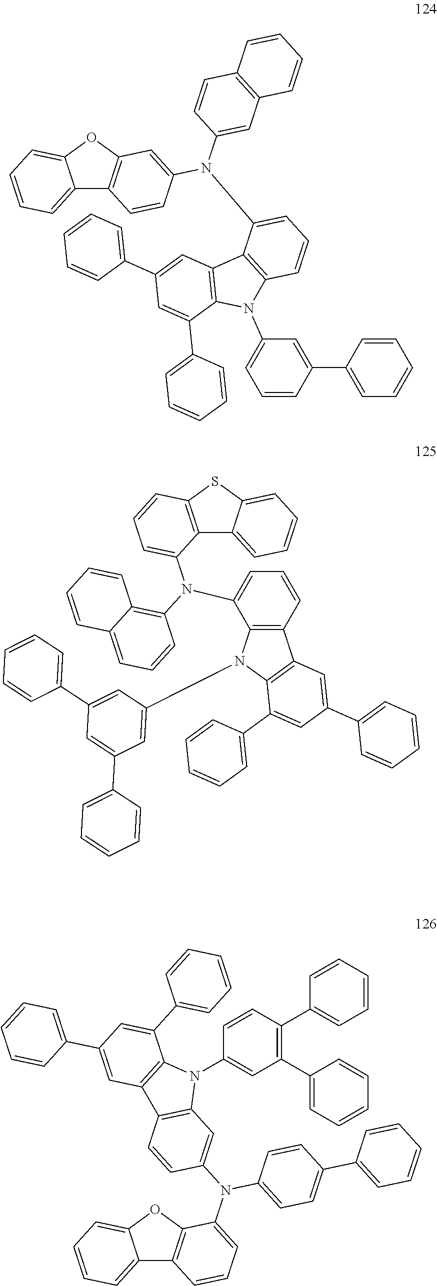 [SMILES: c1ccc(-c2cccc(-n3c4cccc(N(c5ccc6ccccc6c5)c5ccc6c(c5)oc5ccccc56)c4c4cc(-c5ccccc5)cc(-c5ccccc5)c43)c2)cc1 ; c1ccc(-c2cc(-c3ccccc3)cc(-n3c4c(-c5ccccc5)cc(-c5ccccc5)cc4c4cccc(N(c5cccc6ccccc56)c5cccc6sc7ccccc7c56)c43)c2)cc1 ; c1ccc(-c2ccc(N(c3ccc4c5cc(-c6ccccc6)cc(-c6ccccc6)c5n(-c5ccc(-c6ccccc6)c(-c6ccccc6)c5)c4c3)c3cccc4c3oc3ccccc34)cc2)cc1]

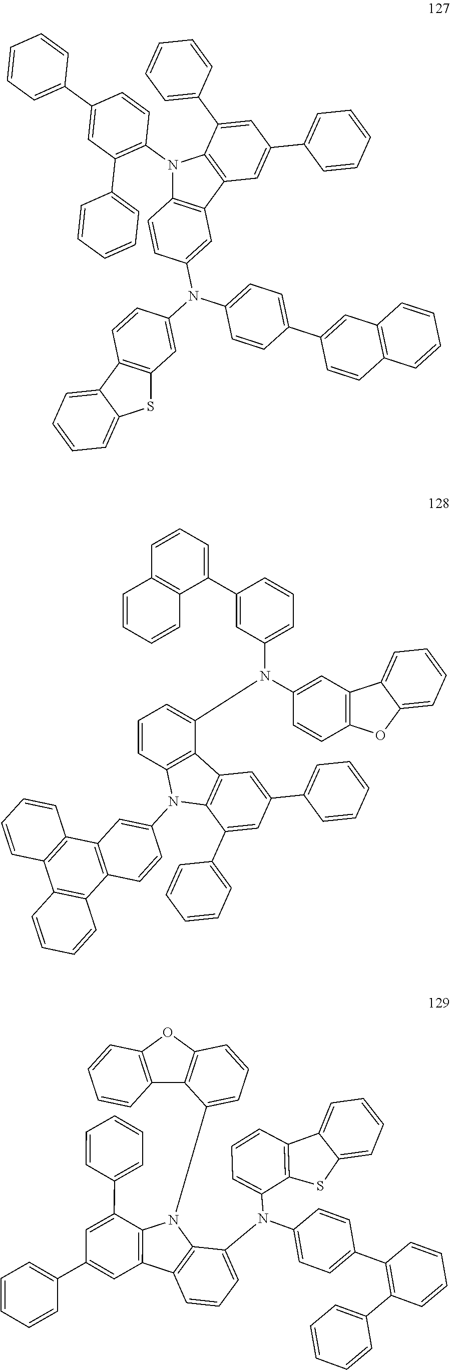 [SMILES: c1ccc(-c2ccc(-n3c4ccc(N(c5ccc(-c6ccc7ccccc7c6)cc5)c5ccc6c(c5)sc5ccccc56)cc4c4cc(-c5ccccc5)cc(-c5ccccc5)c43)c(-c3ccccc3)c2)cc1 ; c1ccc(-c2cc(-c3ccccc3)c3c(c2)c2c(N(c4cccc(-c5cccc6ccccc56)c4)c4ccc5oc6ccccc6c5c4)cccc2n3-c2ccc3c4ccccc4c4ccccc4c3c2)cc1 ; c1ccc(-c2cc(-c3ccccc3)c3c(c2)c2cccc(N(c4ccc(-c5ccccc5-c5ccccc5)cc4)c4cccc5c4sc4ccccc45)c2n3-c2cccc3oc4ccccc4c23)cc1]

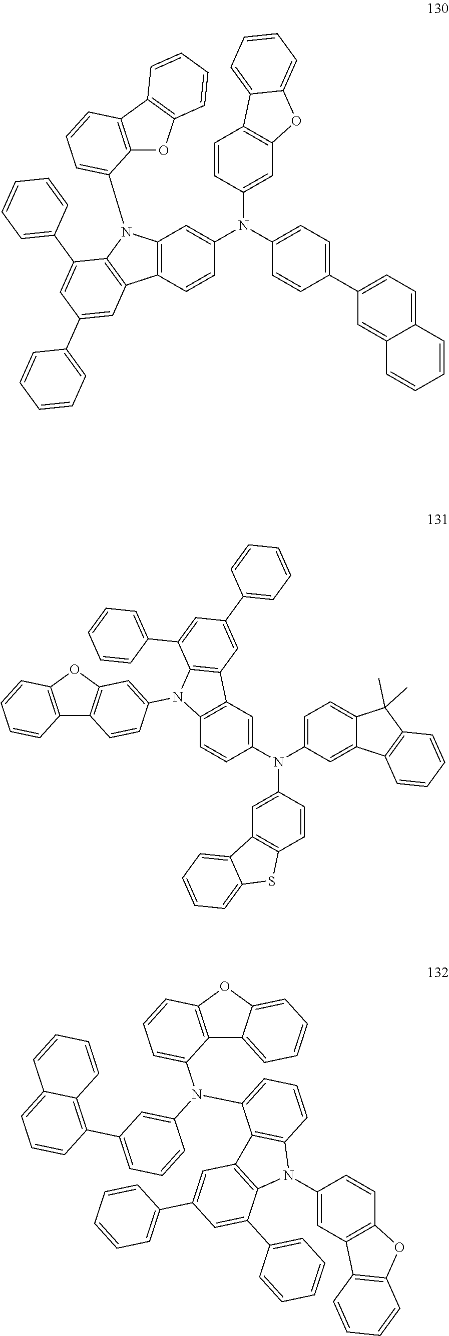 [SMILES: c1ccc(-c2cc(-c3ccccc3)c3c(c2)c2ccc(N(c4ccc(-c5ccc6ccccc6c5)cc4)c4ccc5c(c4)oc4ccccc45)cc2n3-c2cccc3c2oc2ccccc23)cc1 ; CC1(C)c2ccccc2-c2cc(N(c3ccc4sc5ccccc5c4c3)c3ccc4c(c3)c3cc(-c5ccccc5)cc(-c5ccccc5)c3n4-c3ccc4c(c3)oc3ccccc34)ccc21 ; c1ccc(-c2cc(-c3ccccc3)c3c(c2)c2c(N(c4cccc(-c5cccc6ccccc56)c4)c4cccc5oc6ccccc6c45)cccc2n3-c2ccc3oc4ccccc4c3c2)cc1]

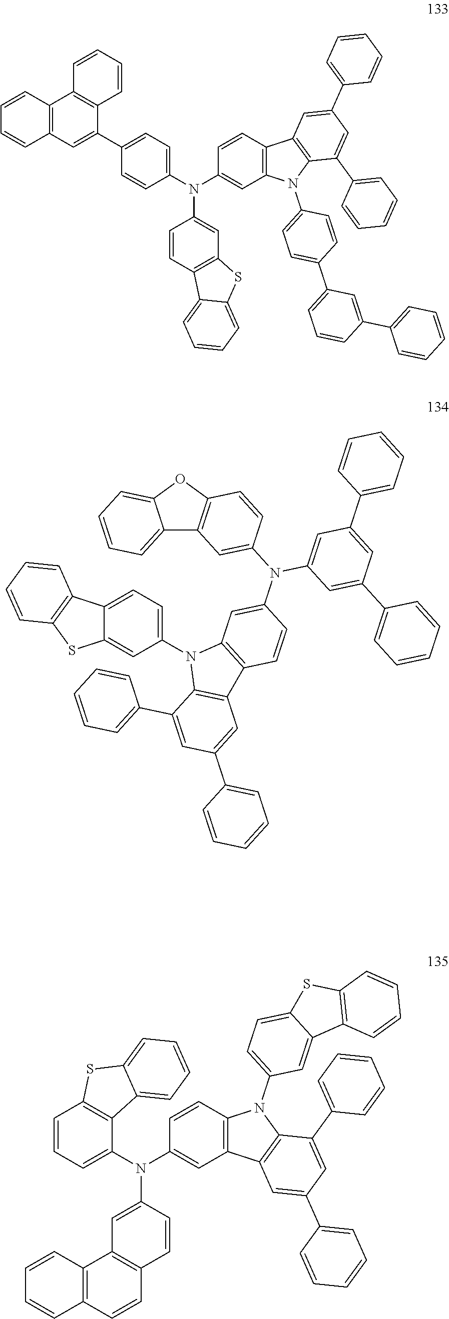 [SMILES: c1ccc(-c2cccc(-c3ccc(-n4c5cc(N(c6ccc(-c7cc8ccccc8c8ccccc78)cc6)c6ccc7c(c6)sc6ccccc67)ccc5c5cc(-c6ccccc6)cc(-c6ccccc6)c54)cc3)c2)cc1 ; c1ccc(-c2cc(-c3ccccc3)cc(N(c3ccc4oc5ccccc5c4c3)c3ccc4c5cc(-c6ccccc6)cc(-c6ccccc6)c5n(-c5ccc6c(c5)sc5ccccc56)c4c3)c2)cc1 ; c1ccc(-c2cc(-c3ccccc3)c3c(c2)c2cc(N(c4ccc5ccc6ccccc6c5c4)c4cccc5sc6ccccc6c45)ccc2n3-c2ccc3sc4ccccc4c3c2)cc1]

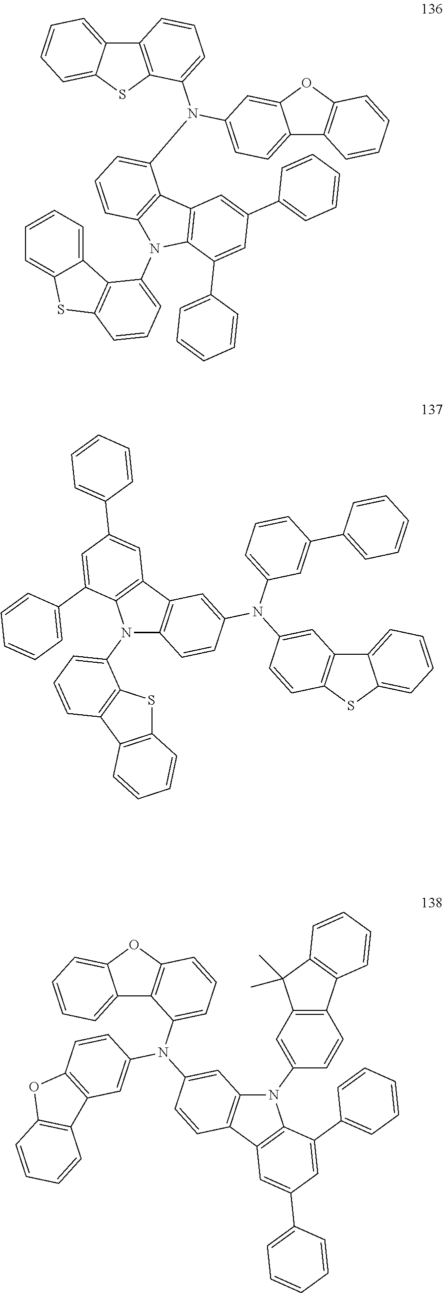 [SMILES: c1ccc(-c2cc(-c3ccccc3)c3c(c2)c2c(N(c4ccc5c(c4)oc4ccccc45)c4cccc5c4sc4ccccc45)cccc2n3-c2cccc3sc4ccccc4c23)cc1 ; c1ccc(-c2cccc(N(c3ccc4sc5ccccc5c4c3)c3ccc4c(c3)c3cc(-c5ccccc5)cc(-c5ccccc5)c3n4-c3cccc4c3sc3ccccc34)c2)cc1 ; CC1(C)c2ccccc2-c2ccc(-n3c4cc(N(c5ccc6oc7ccccc7c6c5)c5cccc6oc7ccccc7c56)ccc4c4cc(-c5ccccc5)cc(-c5ccccc5)c43)cc21]

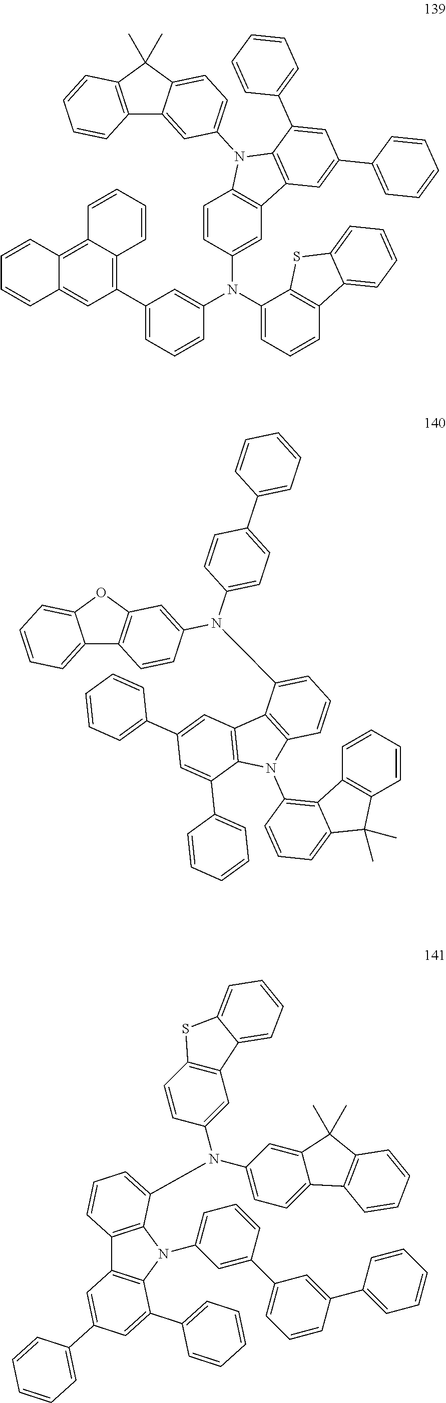 [SMILES: CC1(C)c2ccccc2-c2cc(-n3c4ccc(N(c5cccc(-c6cc7ccccc7c7ccccc67)c5)c5cccc6c5sc5ccccc56)cc4c4cc(-c5ccccc5)cc(-c5ccccc5)c43)ccc21 ; CC1(C)c2ccccc2-c2c(-n3c4cccc(N(c5ccc(-c6ccccc6)cc5)c5ccc6c(c5)oc5ccccc56)c4c4cc(-c5ccccc5)cc(-c5ccccc5)c43)cccc21 ; CC1(C)c2ccccc2-c2ccc(N(c3ccc4sc5ccccc5c4c3)c3cccc4c5cc(-c6ccccc6)cc(-c6ccccc6)c5n(-c5cccc(-c6cccc(-c7ccccc7)c6)c5)c34)cc21]

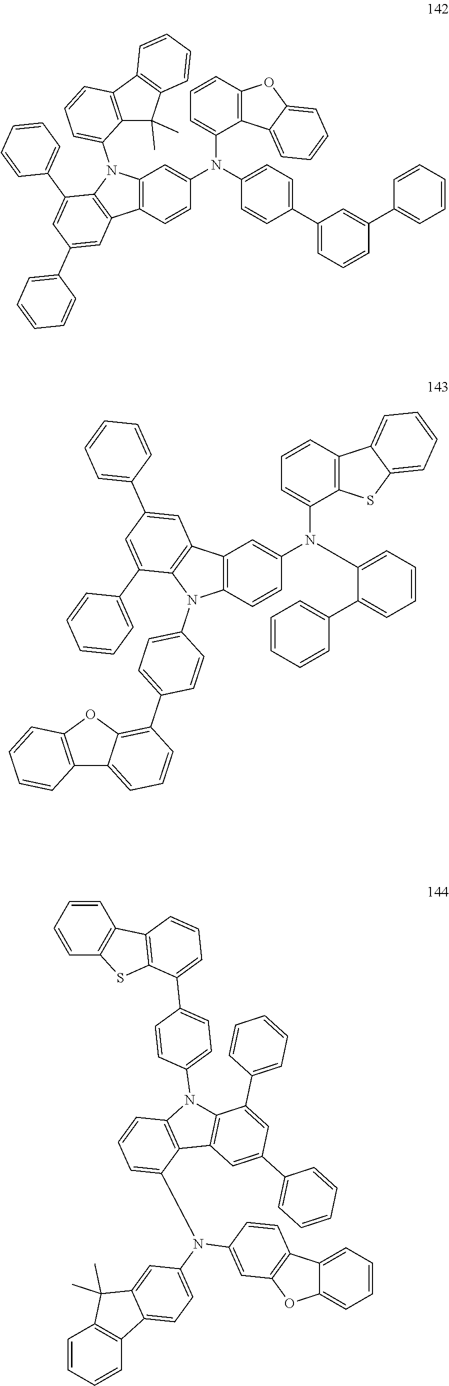 [SMILES: CC1(C)c2ccccc2-c2cccc(-n3c4cc(N(c5ccc(-c6cccc(-c7ccccc7)c6)cc5)c5cccc6oc7ccccc7c56)ccc4c4cc(-c5ccccc5)cc(-c5ccccc5)c43)c21 ; c1ccc(-c2cc(-c3ccccc3)c3c(c2)c2cc(N(c4ccccc4-c4ccccc4)c4cccc5c4sc4ccccc45)ccc2n3-c2ccc(-c3cccc4c3oc3ccccc34)cc2)cc1 ; CC1(C)c2ccccc2-c2ccc(N(c3ccc4c(c3)oc3ccccc34)c3cccc4c3c3cc(-c5ccccc5)cc(-c5ccccc5)c3n4-c3ccc(-c4cccc5c4sc4ccccc45)cc3)cc21]

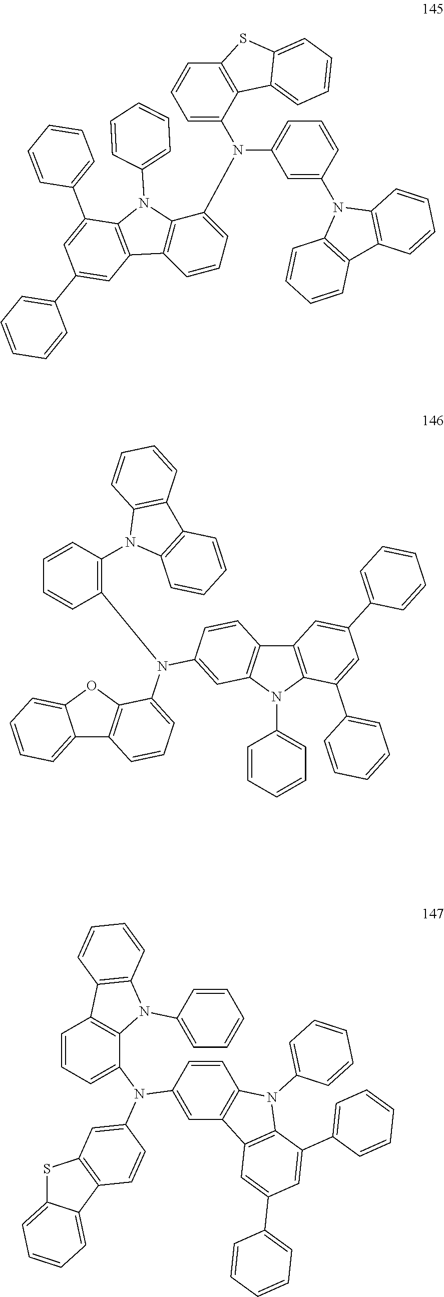 [SMILES: c1ccc(-c2cc(-c3ccccc3)c3c(c2)c2cccc(N(c4cccc(-n5c6ccccc6c6ccccc65)c4)c4cccc5sc6ccccc6c45)c2n3-c2ccccc2)cc1 ; c1ccc(-c2cc(-c3ccccc3)c3c(c2)c2ccc(N(c4ccccc4-n4c5ccccc5c5ccccc54)c4cccc5c4oc4ccccc45)cc2n3-c2ccccc2)cc1 ; c1ccc(-c2cc(-c3ccccc3)c3c(c2)c2cc(N(c4ccc5c(c4)sc4ccccc45)c4cccc5c6ccccc6n(-c6ccccc6)c45)ccc2n3-c2ccccc2)cc1]

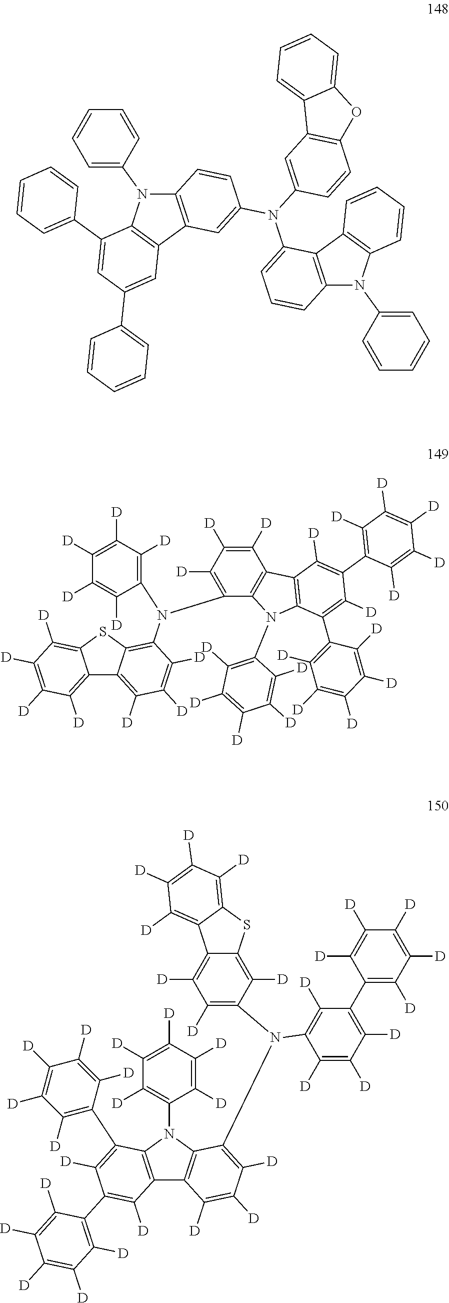

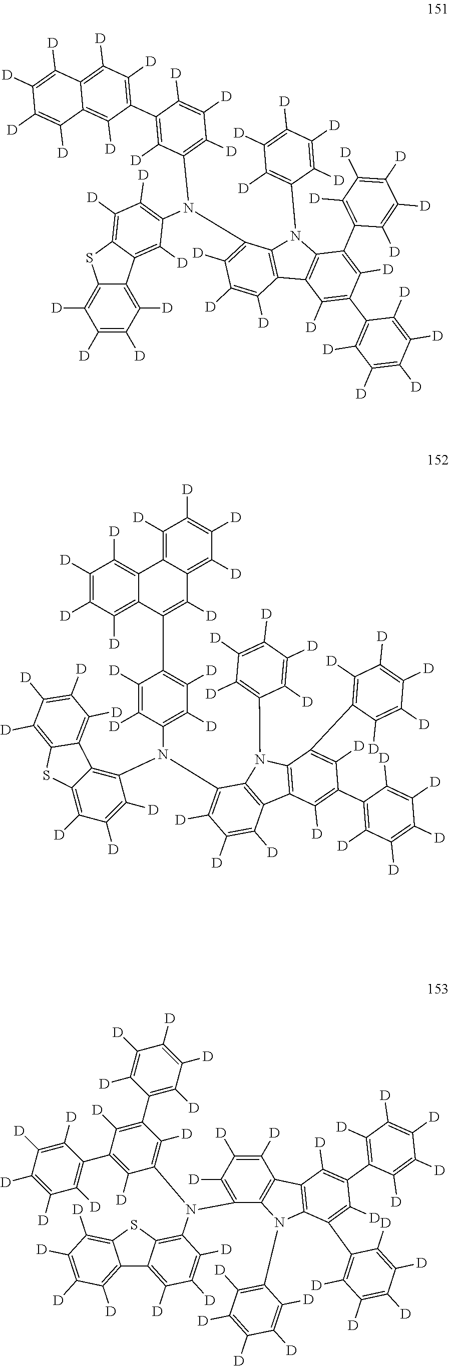

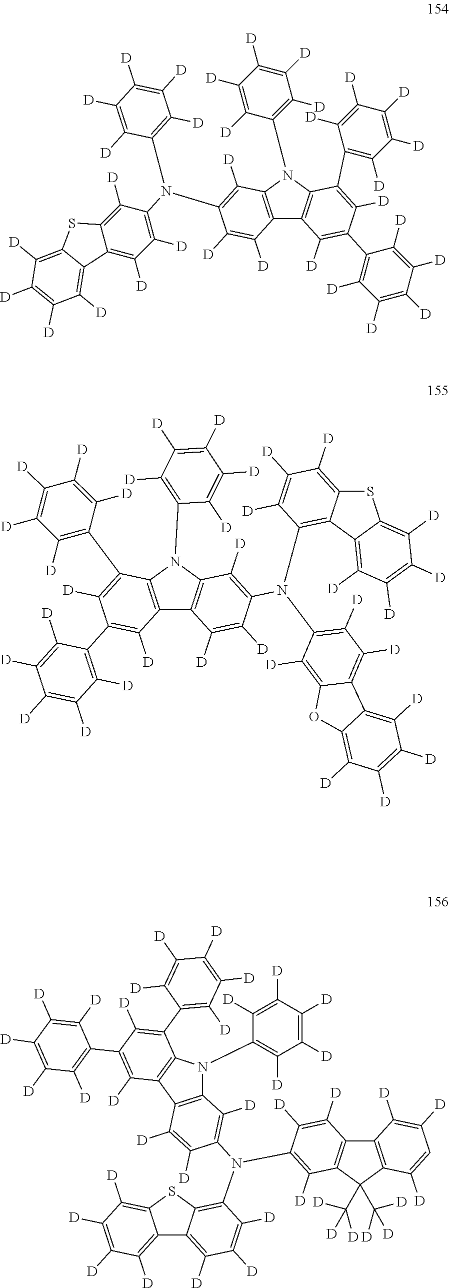

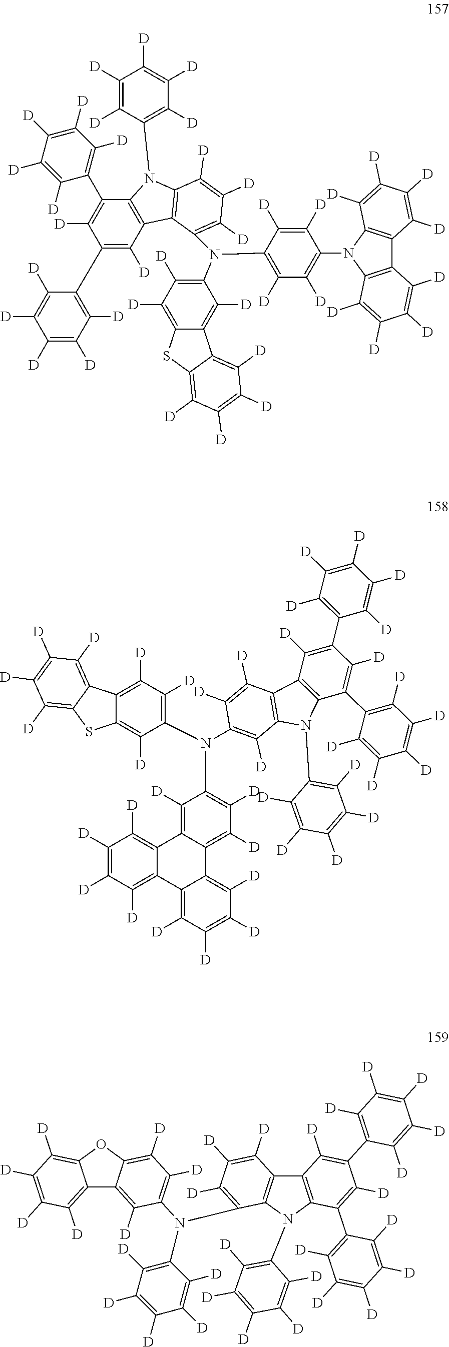

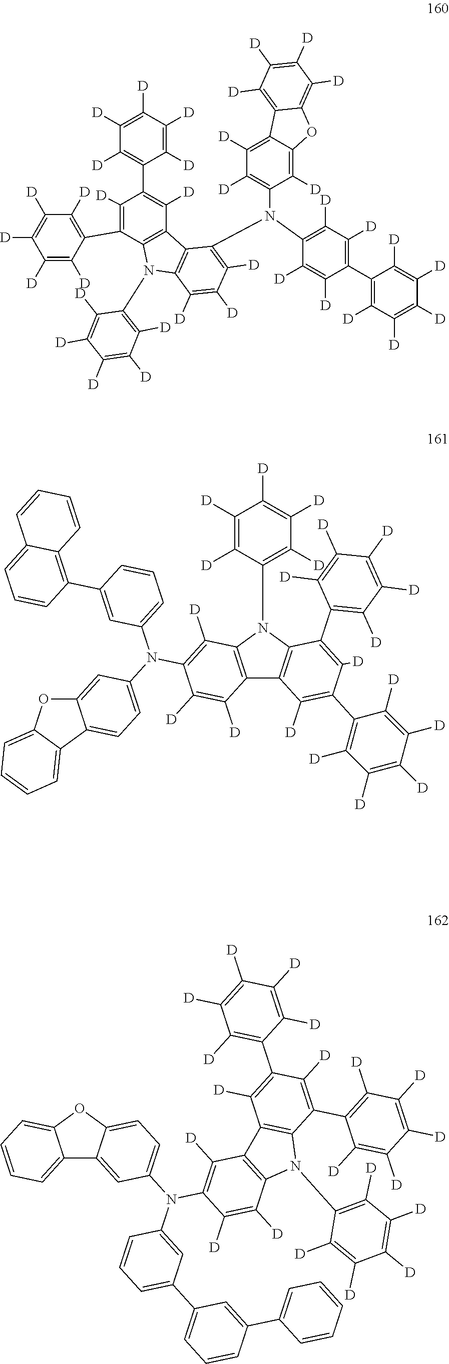

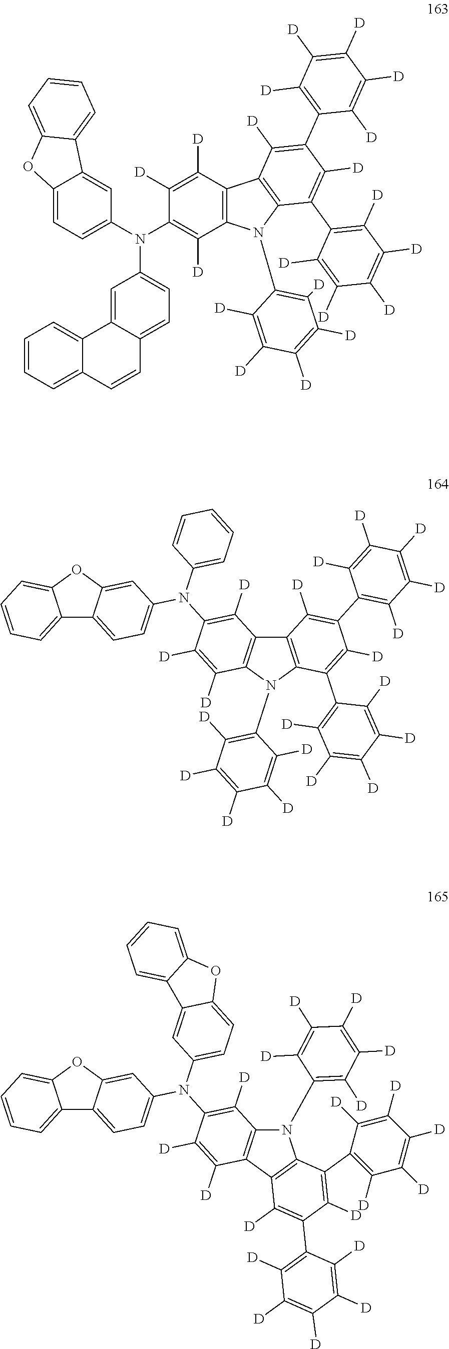

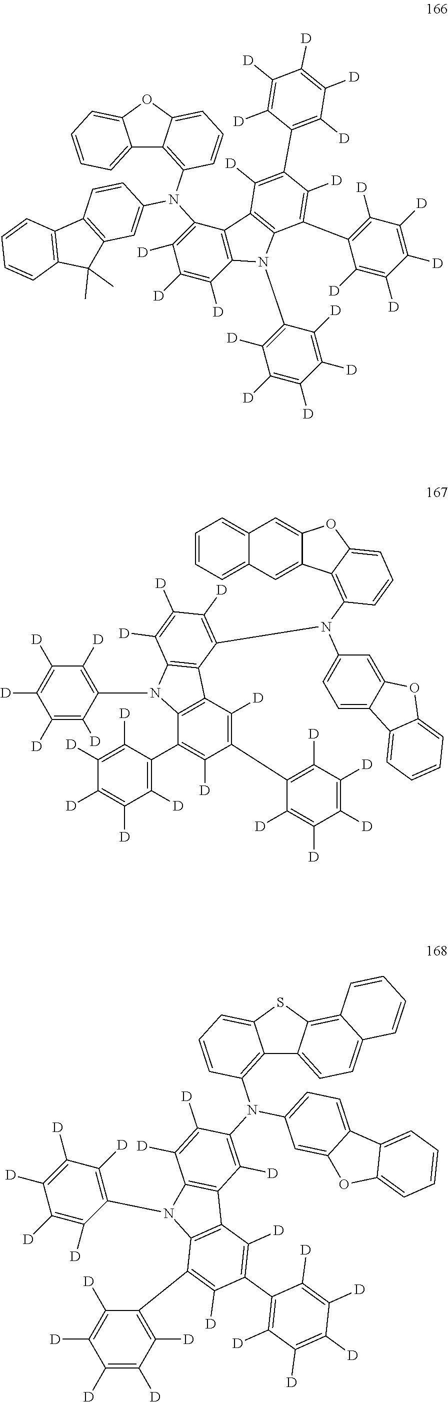

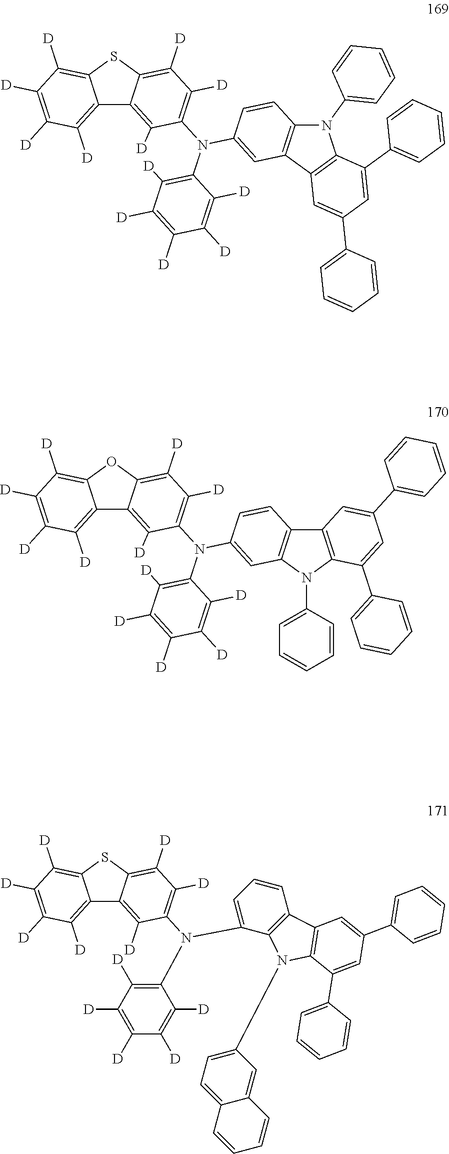 [SMILES: [2H]c1c([2H])c([2H])c(N(c2ccc3c(c2)c2cc(-c4ccccc4)cc(-c4ccccc4)c2n3-c2ccccc2)c2c([2H])c([2H])c3sc4c([2H])c([2H])c([2H])c([2H])c4c3c2[2H])c([2H])c1[2H] ; [2H]c1c([2H])c([2H])c(N(c2ccc3c4cc(-c5ccccc5)cc(-c5ccccc5)c4n(-c4ccccc4)c3c2)c2c([2H])c([2H])c3oc4c([2H])c([2H])c([2H])c([2H])c4c3c2[2H])c([2H])c1[2H] ; [2H]c1c([2H])c([2H])c(N(c2c([2H])c([2H])c3sc4c([2H])c([2H])c([2H])c([2H])c4c3c2[2H])c2cccc3c4cc(-c5ccccc5)cc(-c5ccccc5)c4n(-c4ccc5ccccc5c4)c23)c([2H])c1[2H]]

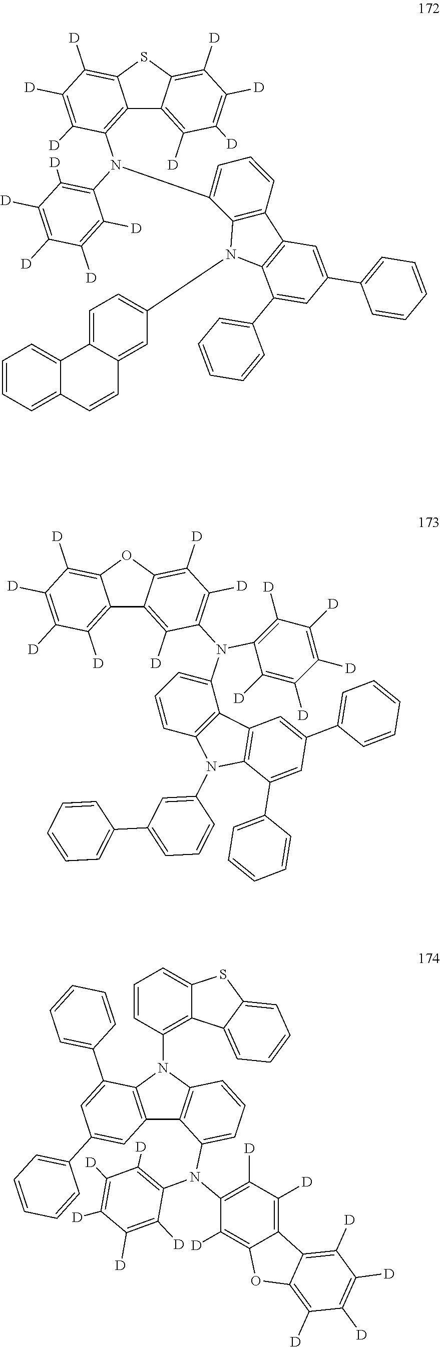 [SMILES: [2H]c1c([2H])c([2H])c(N(c2c([2H])c([2H])c([2H])c3sc4c([2H])c([2H])c([2H])c([2H])c4c23)c2cccc3c4cc(-c5ccccc5)cc(-c5ccccc5)c4n(-c4ccc5c(ccc6ccccc65)c4)c23)c([2H])c1[2H] ; [2H]c1c([2H])c([2H])c(N(c2c([2H])c([2H])c3oc4c([2H])c([2H])c([2H])c([2H])c4c3c2[2H])c2cccc3c2c2cc(-c4ccccc4)cc(-c4ccccc4)c2n3-c2cccc(-c3ccccc3)c2)c([2H])c1[2H] ; [2H]c1c([2H])c([2H])c(N(c2c([2H])c([2H])c3c(oc4c([2H])c([2H])c([2H])c([2H])c43)c2[2H])c2cccc3c2c2cc(-c4ccccc4)cc(-c4ccccc4)c2n3-c2cccc3sc4ccccc4c23)c([2H])c1[2H]]

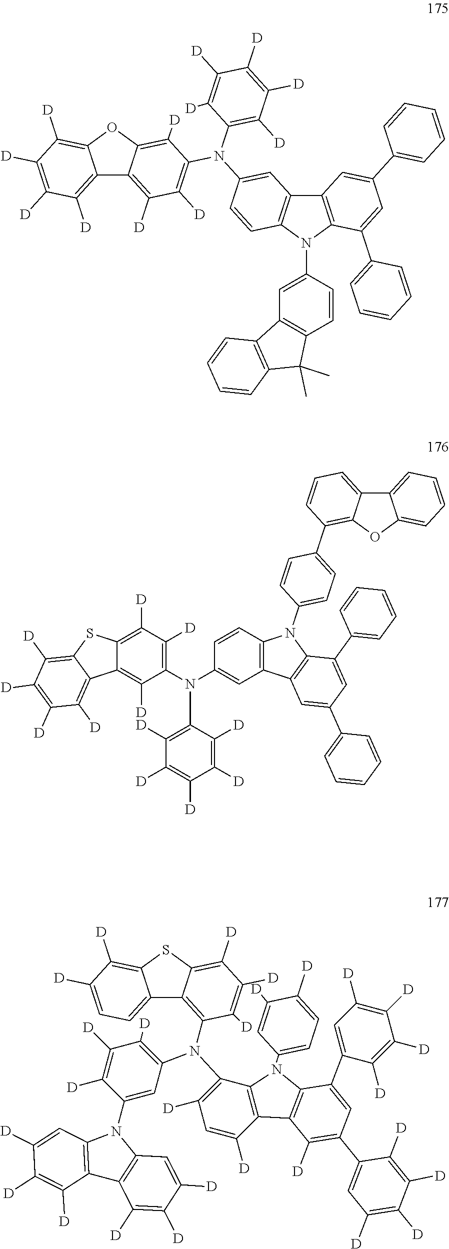

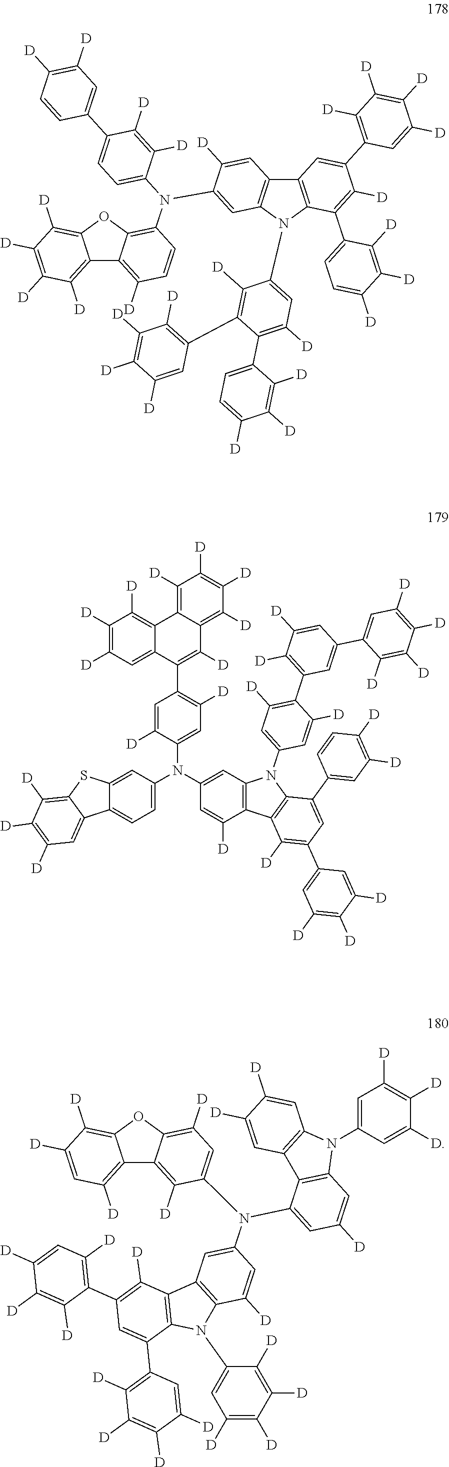

8. An organic light emitting device comprising:
a first electrode;
a second electrode provided to face the first electrode; and
an organic material layer having one or more layers provided between the first electrode and the second electrode,
wherein one or more layers of the organic material layer comprise the heterocyclic compound of claim 1.

9. The organic light emitting device of claim 8, wherein the organic material layer further comprises at least one of a hole transport layer and an electron blocking layer, and at least one of the hole transport layer and the electron blocking layer comprises the heterocyclic compound.

10. The organic light emitting device of claim 8, wherein the organic light emitting device further comprises one or two or more layers selected from the group consisting of an emission layer, a hole injection layer, a hole transport layer, a hole transport auxiliary layer, an electron injection layer, an electron transport layer, an electron blocking layer, and a hole blocking layer.
